(19) United States
(12) Patent Application Publication
(10) Pub. No.: US 2023/0338378 A1
(43) Pub. Date: Oct. 26, 2023

(54) Methods of Administering Myosin Inhibitors

(71) Applicant: MYOKARDIA, INC., Brisbane, CA (US)

(72) Inventors: Vidya V. PERERA, Lawrenceville, NJ (US); Samira MERALI, Lawrenceville, NJ (US); Amy SEHNERT, Brisbane, CA (US); Michael CHEUNG, Brisbane, CA (US); Dewey SETO, Summit, NJ (US); Jeffrey LOCKMAN, Lawrenceville, NJ (US); Marie-Laure PAPI, Lawrenceville, NJ (US)

(21) Appl. No.: 18/306,396

(22) Filed: Apr. 25, 2023

Related U.S. Application Data
(60) Provisional application No. 63/335,028, filed on Apr. 26, 2022.
(60) Provisional application No. 63/335,209, filed on Apr. 26, 2022.
(60) Provisional application No. 63/336,254, filed on Apr. 28, 2022.

(51) Int. Cl. A61K 31/513; A61P 9/00; G16H 20/10

(52) U.S. Cl. CPC A61K 31/513; A61P 9/00; G16H 20/10

(57) ABSTRACT

Methods of administering a myosin inhibitor to a patient and related methods of risk mitigation including controls on distribution are described herein. Methods disclosed herein provide for safe administration of mavacamten and other myosin inhibitors, and mitigate the risk of heart failure due to systolic dysfunction.

### CROSS-REFERENCE TO RELATED APPLICATIONS

This application claims the benefit of and priority to United States provisional application nos. 63/335,028, filed Apr. 26, 2022; 63/335,209, filed Apr. 26, 2022; and 63/336,254, filed Apr. 28, 2022, the entire contents of each of which are incorporated herein by reference.

### TECHNICAL FIELD

The present disclosure relates to methods of administering a myosin inhibitor to a patient and related methods of risk mitigation including controls on distribution.

### BACKGROUND

Hypertrophic cardiomyopathy (HCM) is a heart disease caused by an excess number of myosin-actin cross-bridges, which leads to hypercontractility, and impaired relaxation and compliance. Myosin inhibitors, such as mavacamten, are understood to reduce cardiac muscle contractility by inhibiting excessive myosin-actin cross bridge formation. Myosin inhibitors have been investigated for the treatment of cardiac conditions, including obstructive hypertrophic cardiomyopathy (oHCM), non-obstructive hypertrophic cardiomyopathy (nHCM) and heart failure with preserved ejection fraction (HFpEF). Recently, the myosin inhibitor mavacamten has been shown to provide a clinical benefit in phase 3 clinical trials. Specifically, in the phase 3 EXPLORER-HCM clinical trial, mavacamten treatment was effective in reducing LVOT gradients and improving symptoms, exercise performance and health status in a representative oHCM patient population. (Olivotto et al., 2020, The Lancet, 396(10253), 759-769.) If approved, mavacamten will be the first FDA-approved myosin inhibitor. However, due to the mechanism of action of myosin inhibitors, these drugs must be administered in a manner that mitigates the risk of excess reduction in contractility, which can result in systolic dysfunction and heart failure. Thus, there is a need for methods of administration of myosin inhibitors that maximize the clinical benefits while minimizing risk of adverse events, patient burden, cost, and complexity of administration.

### SUMMARY

The present disclosure relates to methods of safely administering a myosin inhibitor to a patient. Various aspects and embodiments of such methods are described herein. In some embodiments, the methods include a plurality of treatment periods during which the myosin inhibitor is administered to the patient, or optionally during which administration is temporarily discontinued. In some cases, administration may be permanently discontinued. An assessment may be performed at or near the conclusion of a treatment period, and the outcome of the assessment may be used to determine whether the dose administered during the treatment period should be increased, maintained, decreased, or discontinued during the subsequent treatment period. The assessments and corresponding dose adjustments provide for safe and effective administration of the myosin inhibitor. Other aspects of the present disclosure include methods of concomitant administration of other drugs with a myosin inhibitor, and methods for administering myosin inhibitors to avoid drug-drug interactions. Further aspects of the disclosure include methods of controlling the distribution of a myosin inhibitor to mitigate risk.

### BRIEF DESCRIPTION OF THE DRAWINGS

FIG. 1 is a schematic for an initiation phase of a dosing scheme for administering a myosin inhibitor.

FIG. 2 is a schematic for a maintenance phase of a dosing scheme for administering a myosin inhibitor.

FIG. 3 is a schematic for treatment interruption (temporary discontinuation) as part of a dosing scheme for administering a myosin inhibitor

FIG. 4 is a schematic for an initiation phase of an exemplary dosing scheme for administering mavacamten.

FIG. 5 is a schematic for a maintenance phase of an exemplary dosing scheme for administering mavacamten.

FIG. 6 is a schematic for treatment interruption (temporary discontinuation) as part of an exemplary dosing scheme for administering mavacamten.

FIG. 7 is a chart of KCCQ-23 Clinical Summary Score: Mean Change from Baseline Over Time.

FIG. 8 is a chart of KCCQ-23 Clinical Summary Score: Cumulative Distribution of Change from Baseline to Week 30.

FIG. 9 is a chart of HCMSQ Shortness of Breath Domain: Mean Change from Baseline Over Time.

FIG. 10 is a chart of HMCSQ Shortness of Breath Domain: Cumulative Distribution of Change from Baseline to Week 30.

FIG. 11 is an exemplary schedule for echocardiogram assessments, PSF submissions, and dispensing during the first 14 weeks following initiation of myosin inhibitor treatment.

FIG. 12 is an exemplary schedule for echocardiogram assessments and dispensing during the first year following initiation of myosin inhibitor treatment.

FIGS. 13A-D are charts of the time-course of percent of patients simulated under regimen #1 according to Example 2 with (A) LVEF≤50%, (B) VLVOT≤30 mmHg, (C) Mavacamten Plasma Concentration >700 ng/mL, and (D) Mavacamten Plasma Concentration >1000 ng/mL, separated by patient phenotype.

FIGS. 14A-D are charts of the time-course of percent of patients simulated under regimen #2 according to Example 2 with (A) LVEF≤50%, (B) VLVOT≤30 mmHg, (C) Mavacamten Plasma Concentration >700 ng/mL, and (D) Mavacamten Plasma Concentration >1000 ng/mL, separated by patient phenotype.

FIGS. 15A-D are charts of the time-course of percent of patients simulated under regimen #3 according to Example 2 with (A) LVEF≤50%, (B) VLVOT≤30 mmHg, (C) Mavacamten Plasma Concentration >700 ng/mL, and (D) Mavacamten Plasma Concentration >1000 ng/mL, separated by patient phenotype.

FIGS. 16A and 16B are charts of the time-course of percent of patients with LVEF<50% simulated under (A) regimen #1 and (B) regimen #2, according to Example 2, separated by PM and non-PM phenotype.

FIG. 17 shows a Subgroup Analysis of the Primary Composite Functional Endpoint of the clinical study described in Example 1.

FIG. 18 shows the Cumulative Distribution of Change from Baseline to Week 30 in LVOT Peak Gradient of the clinical study described in Example 1.

FIG. 19 shows the Cumulative Distribution of Change from Baseline to Week 30 in pVO2 of the clinical study described in Example 1.

FIGS. 20A and 20B are a steady state mavacamten summary of (A) AUC and (B) Cmax geometric mean ratios from simulations of strong, moderate, and weak CYP2C19 and CYP3A4 inhibition.

FIG. 21 is a schematic view of an example system for authorizing dispensation of medication prescriptions.

FIG. 22 a flowchart of an example arrangement of operations for a method of authorizing dispensation of medication prescriptions.

FIG. 23 is a schematic view of an example computing device that may be used to implement the systems and methods described herein.

FIG. 24 is a flowchart of another example arrangement of operations for a method of authorizing dispensation of medication prescriptions.

### DETAILED DESCRIPTION

### Definitions

While various embodiments and aspects of the present invention are shown and described herein, it will be apparent to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The following documents are incorporated by reference in their entirety:
The American Society of Echocardiography, Recommendations for Cardiac Chamber Quantification in Adults: A Quick Reference Guide from the ASE Workflow and Lab Management Task Force, July 2018
Lang et al., Recommendations for Cardiac Chamber Quantification by Echocardiography in Adults: An Update from the American Society of Echocardiography and the European Association of Cardiovascular Imaging, Journal of the American Society of Echocardiography, January 2015
Nagueh et al., Recommendations for the Evaluation of Left Ventricular Diastolic Function by Echocardiography: An Update from the American Society of Echocardiography and the European Association of Cardiovascular Imaging, Journal of the American Society of Echocardiography, 2016; 29:277-314
Caballero et al., Echocardiographic reference ranges for normal cardiac Doppler data: results from the NORRE Study, European Heart Journal—Cardiovascular Imaging (2015) 16, 1031-1041
Jozine M. ter Maaten et al., Connectin heart failure with preserved ejection fraction and renal dysfunction: the role of endothelial dysfunction and inflammation, European Journal of Heart Failure (2016) 18, 588-598
ATS/ACCP Statement on Cardiopulmonary Exercise Testing, American Thoracic Society/American College of Chest Physicians, Nov. 1, 2001
Zaid et al., Pre- and Post-Operative Diastolic Dysfunction in Patients with Valvular Heart Disease, Journal of the American College of Cardiology, 2013, 62(21), 1922-1930
Gupta et al., Racial differences in circulating natriuretic peptide levels: the atherosclerosis risk in communities study, Journal of the American Heart Association, 2015; 4:e001831
Eugene Braunwald, Cardiomyopathies: An Overview, Circ Res. 2017; 121:711-721
Towbin and Jefferies, Cardiomyopathies Due to Left Ventricular Noncompaction, Mitochondrial and Storage Diseases and Inborn Errors of Metabolism, Circ Res. 2017; 121:838-854
Cirino and Ho, Hypertrophic Cardiomyopathy Overview. 2008. In: Adam et al., eds., GeneReviews®, Seattle (WA): University of Washington, Seattle; 1993-2020.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, “about” means a range extending to +/−10% of the specified value. In some embodiments, “about” means the specified value.

As used herein, “treatment” or “treating,” or “ameliorating” are used interchangeably herein. These terms refer to an approach for obtaining beneficial or desired results including but not limited to a therapeutic benefit. Therapeutic benefit means eradication or amelioration of the underlying disorder being treated and/or eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance. For example, certain methods described herein treat hypertrophic cardiomyopathy (HCM) by decreasing or reducing the occurrence or progression of HCM; or treat HCM by decreasing a symptom of HCM. Symptoms of, or test results indicating HCM would be known or may be determined by a person of ordinary skill in the art and may include, but are not limited to, shortness of breath (especially during exercise), chest pain (especially during exercise), fainting (especially during or just after exercise), sensation of rapid, fluttering or pounding heartbeats, atrial and ventricular arrhythmias, heart murmur, hypertrophied and non-dilated left ventricle, thickened heart muscle, thickened left ventricular wall, elevated pressure gradient across left ventricular outflow tract (LVOT), and elevated post-exercise or Valsalva LVOT gradient.

“Patient” or “subject” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, cats, monkeys, goat, sheep, cows, deer, and other non-mammalian animals. In some embodiments, a patient or subject is a human. In some embodiments, the patient is suffering from obstructive HCM.

As used herein, “administration” of a disclosed compound encompasses the delivery to a subject of a compound as described herein, or a prodrug or other pharmaceutically acceptable derivative thereof, using any suitable formulation or route of administration, e.g., as described herein. In some embodiments, administration is oral administration.

As used herein, “near the conclusion of” with respect to a treatment period, refers to a portion of a treatment period that is more than half-way through the treatment period and within about two weeks of the end of the treatment period. In some embodiments, near the conclusion of the treatment period is more than half-way through the treatment period and within about 1 week (e.g., within about 3 days, within about 1 day) of the end of the treatment period. In some embodiments, near the conclusion of the treatment period is within +/−about two weeks of the end of the treatment period. In some embodiments, near the conclusion of the treatment period is within +/−about one week of the end of the treatment period. In some embodiments, near the conclusion of the treatment period is more than half-way through the treatment period and within the last two weeks of the treatment period. In some embodiments, near the conclusion of a treatment period is the final week of the treatment period, e.g., week 4 of a 4-week treatment period.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

Myosin inhibitors are being investigated for the treatment of cardiac conditions, including obstructive hypertrophic cardiomyopathy (oHCM), non-obstructive hypertrophic cardiomyopathy (nHCM) and heart failure with preserved ejection fraction (HFpEF). While myosin inhibitors have been shown to provide a clinical benefit, for example by reducing left ventricular outflow tract obstruction, they also present a risk of excessive reduction in left ventricular (LV) contractility due to their mechanism of action. Excessive reduction in LV contractility generally results in systolic dysfunction, e.g., a left ventricular ejection fraction (LVEF) below 50%, which can result in heart failure and death. Reduced LVEF can be caused by a myosin inhibitor when the plasma concentration of the myosin inhibitor exceeds the therapeutic range. Many pharmacokinetic factors contribute to the plasma concentration of a drug, including the dose administered and the rate of metabolism. As an example, mavacamten is primarily metabolized by the CYP2C19 enzyme. Some individuals have mutations in their CYP2C19 enzymes, which cause them to metabolize mavacamten at different rates, thereby affecting plasma concentration. Individuals can be grouped as poor metabolizers, intermediate metabolizers, normal metabolizers, rapid metabolizer, and ultra-rapid metabolizers based on mutations in CYP2C19. As an example, a CYP2C19 poor metabolizer receiving a daily dose of 5 mg of mavacamten for a period of weeks, due to a slower rate of metabolism of mavacamten, may achieve a high blood plasma concentration of mavacamten that is above the therapeutic range and which presents a high risk of adverse events. As another example, a CYP2C19 ultra-rapid metabolizer receiving a daily dose of 5 mg of mavacamten for a period of weeks, due to a faster rate of metabolism of mavacamten, may have a low blood plasma concentration of mavacamten that is below the therapeutic range and presents a reduced likelihood of therapeutic benefit (e.g., reduction in LVOT gradient). The metabolism of mavacamten and other myosin inhibitors can therefore vary across an intended patient population. There is a need for methods of administration of myosin inhibitors that maximize the clinical benefits while minimizing risk of adverse events, patient burden, cost and complexity of administration.

Disclosed herein are methods of treating cardiac conditions. Certain methods disclosed herein mitigate the risk of heart failure and systolic dysfunction during such treatment. In some embodiments, the risk is reduced, e.g., by at least about 10%, 20%, 30%, 40%, 50%, 60%, 70%, 80%, 90%, or more compared to other methods of administration. The methods may include particular methods of administration of the myosin inhibitor, including dose adjustments (decreases and/or increases), and particular methods of assessment, such as echocardiography, assessment of left ventricular outflow tract obstruction, and assessment of LVEF, which may be used to guide the administration of the myosin inhibitor. In some embodiments, the methods of the present disclosure mitigate, manage, reduce, or lower the risk of such adverse events.

In some embodiments, an aspect of the present methods comprises determining LVOT gradient, or another measure of left ventricular outflow tract obstruction, at one or more assessments after beginning treatment with a myosin inhibitor, and adjusting the dose as needed based on such assessments. LVOT gradient is used as a measure of therapeutic benefit for treatment of oHCM and related cardiac conditions characterized by left ventricular outflow tract obstruction. When LVOT gradient decreases quickly upon initiation of myosin inhibitor therapy, it can be inferred that the patient's exposure to the myosin inhibitor is high. High levels of exposure to a myosin inhibitor present a risk of systolic dysfunction and heart failure. According to aspects of the present disclosure, by assessing LVOT gradient (or another measure of left ventricular outflow tract obstruction) at or near the conclusion of two or more separate treatment periods during an initiation phase, and reducing the dose of the myosin inhibitor administered based on a low LVOT gradient at those assessments, the risk of systolic dysfunction and heart failure is mitigated. As a particular example, the risk of systolic dysfunction and heart failure can be mitigated by assessing Valsalva LVOT gradient at Weeks 4 and 8 following initial administration of a myosin inhibitor (e.g, mavacamten), and reducing the dose of myosin inhibitor (e.g., mavacamten) administered following Weeks 4 and 8 when the Valsalva LVOT gradient is below a threshold (e.g., 20 mmHg). This criteria uses exaggerated pharmacological effect during the early stages of treatment to prospectively reduce dose in patients who have an increased likelihood of subsequent reduction in LVEF, prior to experiencing an episode of LVEF reduction.

The dose adjustments based on two or more LVOT assessments can further be combined with methods involving determining LVEF at one or more assessments after (and optionally before) beginning treatment with a myosin inhibitor, and modifying treatment (e.g., by temporary discontinuation) based on LVEF. Including LVEF assessments may provide further risk mitigation for the myosin inhibitor treatment. LVEF is a direct measure of systolic dysfunction, which can lead to heart failure. Using both LVEF and LVOT gradient provides two measurements during initiation of myosin inhibitor therapy to mitigate risk of systolic dysfunction and heart failure. Both LVOT gradient and LVEF can be determined using a non-invasive technique, such as a non-invasive imaging technique (e.g, echocardiography, cardiac magnetic resonance imaging). Where a non-invasive technique (e.g., imaging technique, echocardiography) is used for determining LVOT gradient and LVEF, the need for other procedures, including invasive procedures, may be eliminated. For example, the need for determining blood plasma concentration (e.g., a “trough” measurement) may be eliminated.

The use of the one or more assessments also allows for administration to a broad patient population, for example by allowing for use of a “unified posology” regardless of patient genotype. Different patients will have different responses to a myosin inhibitor and as a result, some will be at greater risk of adverse event. For example, different exposure levels in different patients may put some patients at greater risk of an adverse event. In particular, patients who are poor metabolizers of a myosin inhibitor (e.g., due to a CYP2C19 (for mavacamten) or a CYP2D6 (for aficamten) poor metabolizer phenotype) will experience higher, and potentially dangerous exposure levels at a given dose, where that given dose may be the ideal starting dose for a large patient population, such as intermediate metabolizers, normal metabolizers, rapid metabolizers, and/or ultra-rapid metabolizers. By providing the potential for dose reduction and temporary discontinuation, based on relevant assessment outcomes, particularly during initial treatment (“initiation phase”), all patients can begin treatment at the same starting dose, and without the need for costly or time-consuming genotyping assays. Patients who will mitigate risk by decreasing the dose administered or temporarily discontinuing administration can be identified in a timely manner by the assessments (e.g., LVOT and/or LVEF) during an initiation phase, and are given a dose reduction or temporary discontinuation before exposure is too high. Patients who are at low risk of adverse event are able to maintain a higher dose during the initiation phase under the same dosing scheme, rather than receiving a lower, potentially less effective dose.

In some embodiments, the present methods comprise a method of mitigating, managing, reducing, or lowering the risk of an adverse event to myosin inhibitor therapy.

In some embodiments, the present methods comprise a method of mitigating, managing, reducing, or lowering the risk of an adverse event due to myosin inhibitor therapy.

In some embodiments, the present methods comprise a method of mitigating, managing, reducing, or lowering the risk of heart failure during myosin inhibitor therapy.

In some embodiments, the present methods relate to a method of mitigating, managing, reducing, or lowering the risk of systolic dysfunction during myosin inhibitor therapy.

In some embodiments, the present methods relate to a method of mitigating, managing, reducing, or lowering the risk of heart failure due to systolic dysfunction during myosin inhibitor therapy.

In some embodiments, the risk is mitigated, managed, reduced or lowered during an initiation phase by providing for dose reduction and/or treatment interruption (temporary discontinuation) during the initiation phase. The terms temporary discontinuation and treatment interruption are used interchangeably herein.

In some embodiments, the risk is mitigated, managed, reduced or lowered during a maintenance phase by providing for dose adjustment and/or treatment interruption (temporary discontinuation) during the maintenance phase.

In some embodiments, an initiation phase is from about 2 weeks to about 36 weeks. In some embodiments, an initiation phase is from about 4 weeks to about 24 weeks. In some embodiments, an initiation phase is from about 4 weeks to about 16 weeks (e.g, 12 week). In some embodiments, an initiation phase is from about 8 weeks to about 24 weeks. In some embodiments, an initiation phase is from about 8 weeks to about 16 weeks. In some embodiments, an initiation phase is from about 4 weeks to about 12 weeks. In some embodiments, an initiation phase is about 12 weeks. In some embodiments, an initiation phase is about 8 weeks. In some embodiments, an initiation phase is about 6 weeks. In some embodiments, an initiation phase is about 4 weeks. In some embodiments, an initiation phase is about 16 weeks.

In some embodiments, the present methods comprise a method of treating a disease, or a method of administering a myosin inhibitor while mitigating, managing, reducing, or lowering the risk of an adverse event to myosin inhibitor therapy (e.g., heart failure, systolic dysfunction, or heart failure due to systolic dysfunction).

The present methods are useful for treating a patient with a cardiac condition, heart disease, cardiovascular disease, or symptom(s) thereof, using a myosin inhibitor. The methods are useful across a diverse population of patients with different cardiac conditions and different characteristics, genotypes, and phenotypes.

In some embodiments, the patient is a poor metabolizer of a myosin inhibitor (e.g., mavacamten). In some embodiments, the patient is a normal metabolizer of a myosin inhibitor (e.g., mavacamten). In other embodiments, the patient is an intermediate, rapid, or ultra-rapid metabolizer of a myosin inhibitor (e.g., mavacamten). Methods of the present disclosure may provide for administration of a myosin inhibitor to a patient regardless of the patient's relative ability to metabolize a myosin inhibitor, such as mavacamten. The dosing methodology and assessments provide for safe administration of myosin inhibitors (e.g., mavacamten) across a diverse patient population, including poor metabolizers, intermediate metabolizers, normal metabolizers, rapid metabolizers, and ultra-rapid metabolizers. The dosing methodology also provides for initiation of administration without the need for conducting a genotyping assay to determine the metabolizer genotype of a patient, which may be costly and time-consuming. This unified posology allows for timely assessment of patient response in the clinic, reduced cost and reduced complexity of administration. For example, doctors, pharmacists, and other involved parties, do not need to be trained and certified regarding genotyping protocols since genotyping is not required nor regarding multiple genotype-specific dosing methologies, which can add further complexity and risk for medication errors. Methods of the present disclosure may also provide for administration of a myosin inhibitor to a patient regardless of the patient's body weight.

Poor metabolizers of a myosin inhibitor (e.g., mavacamten) can include individuals with CYP2C19 polymorphisms. In some embodiments, the poor metabolizers of the myosin inhibitor (e.g., mavacamten) are of Asian descent. In some such cases, the poor metabolizers are of south Asian descent. In some embodiments, Asian descent includes, but not limited to, Japanese population, Chinese population, Thai population, Korean population, Filipino population, Indonesian population, and Vietnamese population. In some embodiments, the poor metabolizers of the myosin inhibitor (e.g., mavacamten) are not of Asian descent.

In some embodiments, the poor metabolizer of the myosin inhibitor (e.g., mavacamten) has a CYP2C19 poor metabolizer genotype. In some embodiments, the poor metabolizer of the myosin inhibitor (e.g., mavacamten) has a CYP2C19 *2/*2, *2/*3, or *3/*3 genotype.

In some embodiments, the poor metabolizer of the myosin inhibitor (e.g., mavacamten) is an Asian descendant. In some embodiments, the poor metabolizer of the myosin inhibitor (e.g., mavacamten) is a Japanese descendant.

Poor metabolizers of a myosin inhibitor (e.g., aficamten) can include individuals with CYP2D6 polymorphisms. In some embodiments, the poor metabolizer of the myosin inhibitor (e.g., aficamten) has a CYP2D6 poor metabolizer genotype.

In some embodiments, the patient, treated by a method described herein, is diagnosed with and/or suffering from a cardiac condition selected from the group consisting of hypertrophic cardiomyopathy (HCM), diastolic dysfunction, left ventricular hypertrophy, malignant left ventricular hypertrophy, angina, ischemia, restrictive cardiomyopathy (RCM), heart failure with preserved ejection fraction (HFpEF), and combinations thereof. In some instances, the patient, treated by a method described herein, is diagnosed with and/or suffering from HCM and/or HFpEF.

In some embodiments, the patient, treated by a method described herein, is diagnosed with and/or suffering from obstructive HCM (oHCM). In some such cases, the patient is diagnosed with and/or suffering from symptomatic NYHA class II-III obstructive HCM. In some such cases, the patient is an adult. In other cases, the patient is a pediatric patient.

The NYHA functional classification grades the severity of heart failure symptoms as one of four functional classes. The NYHA functional classification is widely used in clinical practice and in research because it provides a standard description of severity that can be used to assess response to treatment and to guide management. The NYHA functional classification based on severity of symptoms and physical activity are:
Class I: No limitation of physical activity. Ordinary physical activity does not cause undue breathlessness, fatigue, or palpitations
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

In some embodiments, the patient is diagnosed with and/or suffering from HFpEF.

In some embodiments, the patient is suffering from a symptom of a cardiovascular disease, e.g., shortness of breath, dizziness, chest pain, syncope, or a limit on an activity of daily living (e.g., limit on personal care, mobility, or eating).

In some embodiments, the patient is diagnosed with and/or suffering from a condition (e.g., a cardiac condition) selected from valvular aortic stenosis, mixed LV systolic and diastolic dysfunction, idiopathic RV hypertrophy, chronic kidney disease, aortic insufficiency, tetralogy of Fallot, mitral stenosis, Noonan Syndrome, or acute coronary syndrome.

In some embodiments, the patient has normal systolic contractility or systolic hypercontractility, wherein the left ventricular ejection fraction of the patient is >50%.

In some embodiments, the patient has any one or combination of myocardial diastolic dysfunction, an elevated left ventricular filling pressure, left ventricular hypertrophy and left atrium enlargement (LAE).

Diastolic dysfunction is present or an important feature of a series of diseases including, but not limited to, hypertrophic cardiomyopathy (HCM), heart failure with preserved ejection fraction (HFpEF), left ventricular hypertrophy (LVH)—including both disorders of active relaxation and disorders of chamber stiffness (diabetic HFpEF). Diastolic dysfunction may be diagnosed using one or more techniques and measurements, including: catheter procedures, E/e′, left atrial size, and BNP or NT-proBNP.

Individuals with HCM can be subdivided based on the presence or absence of left ventricular outflow tract obstruction (LVOT). The presence of LVOT obstruction, i.e. obstructive HCM (oHCM) is associated with more severe symptoms and greater risk of heart failure and cardiovascular death. Limited data support medical treatments (beta blockers, calcium channel blockers, disopyramide) in this patient subset, and persistently symptomatic patients may be referred for invasive septal reduction therapy.

Ejection fraction is an indicator of normal or hypercontractile systolic function, i.e., ejection fraction is greater than about 52% or 50% in patients with normal or hypercontractile systolic function.

LVH, which is characterized by wall thickness, may be diagnosed using one or more techniques and measurements, including: echocardiogram, cardiac MRI, noninvasive imaging techniques (e.g., tissue Doppler imaging) and E/e′.

Patients in need of treatment for diastolic dysfunction include patients from a patient population characterized by oHCM, nHCM, LVH, or HFpEF. Patients in need of treatment for diastolic dysfunction include patients who exhibit left ventricle stiffness as measured by echocardiography or left ventricle stiffness as measured by cardiac magnetic resonance.

In some embodiments, the patient in need thereof exhibits left ventricle stiffness as measured by echocardiography.

Further determining factors for diagnosing diastolic dysfunction using echocardiography are described in J. Am Soc Echocardiogr. 29(4):277-314 (2016), the entire contents of which are incorporated herein by reference for all purposes.

In some embodiments, the patient in need thereof exhibits left ventricle stiffness as measured by cardiac magnetic resonance. Cardiac magnetic resonance is used to determine peak filling rate, time to peak filling, and peak diastolic strain rate. Accordingly, in some embodiments, a patient has left ventricle stiffness as measured by cardiac magnetic resonance when at least one of the following characteristics are met: abnormal peak filing rate, time to peak filling, or peak diastolic strain rate.

In some embodiments, the patient in need thereof is suffering from diastolic dysfunction, left ventricular hypertrophy, left ventricular outflow tract obstruction, increased left ventricular wall thickness (or mass index), increased interventricular septal (IVS) wall thickness, poor or reduced cardiac elasticity, poor or reduced diastolic left ventricular relaxation, abnormal high left atrial pressure, reduced E/e′ ratio, diminished exercise capacity or tolerance, diminished peak oxygen consumption (VO₂), increased left ventricular diastolic pressure, or any combination thereof.

In some embodiments, the patient in need thereof is suffering from hypertrophic cardiomyopathy (HCM) characterized by at least one biomarker selected from elevated level of NT-proB-Type Natriuretic Peptide (NT-proBNP), elevated level of cardiac troponin I. In another embodiment, the HCM patient in need thereof has a predisposition to developing HCM.

In some embodiments, the patient in need thereof are suffering from chest pain, dyspnea, angina, syncope or dizziness.

In some cases, patients may be at risk of high exposure levels of a myosin inhibitor when the myosin inhibitor is concomitantly administered with a compound that alters the activity of one or more cytochrome P450 (CYP) enzymes (e.g., CYP inducer, CYP inhibitor). For example, a patient receiving a myosin inhibitor that is predominantly metabolized by CYP2C19 may experience levels of exposure to the myosin inhibitor that are too high and unsafe when concomitantly administered a strong CYP2C19 inhibitor. In some instances, patients may be at risk of below efficacious exposure levels of a myosin inhibitor when the myosin inhibitor is concomitantly administered with a compound that alters the activity of one or more CYP enzymes (e.g., CYP inducer, CYP inhibitor). For example, a patient receiving a myosin inhibitor that is predominantly metabolized by CYP2D6 may experience a below efficacious exposure of the myosin inhibitor when concomitantly administered a strong CYP2D6 inducer. In some instances, the risk of exposures of the myosin inhibitor that are too high or too low may occur when a patient who is receiving concomitant administration of the myosin inhibitor and a compound that alters the activity of one or CYP enzymes, changes the dose of the compound (e.g., discontinues administration of the compound). The present disclosure includes methods of concomitant administration of other drugs with a myosin inhibitor that mitigate, manage, reduce, and/or lower the aforementioned risks.

In some embodiments, the patient is concomitantly administered a weak CYP2C19 inhibitor or a moderate CYP3A4 inhibitor during myosin inhibitor therapy (e.g., with mavacamten). For example, in some embodiments, the patient is not receiving a weak CYP2C19 inhibitor or a moderate CYP3A4 inhibitor, but subsequently initiates administration of a weak CYP2C19 inhibitor or a moderate CYP3A4 inhibitor during myosin inhibitor therapy. In some embodiments, the weak CYP2C19 inhibitor or moderate CYP3A4 inhibitor is selected from the group consisting of cimetidine, ciprofloxacin, diltiazem, felbamate, omeprazole at a dose of 20 mg once daily, isoniazid, fluconazole, and verapamil.

In some embodiments, the patient is concomitantly administered a weak CYP2D6 inhibitor or inducer during myosin inhibitor therapy (e.g., with aficamten).

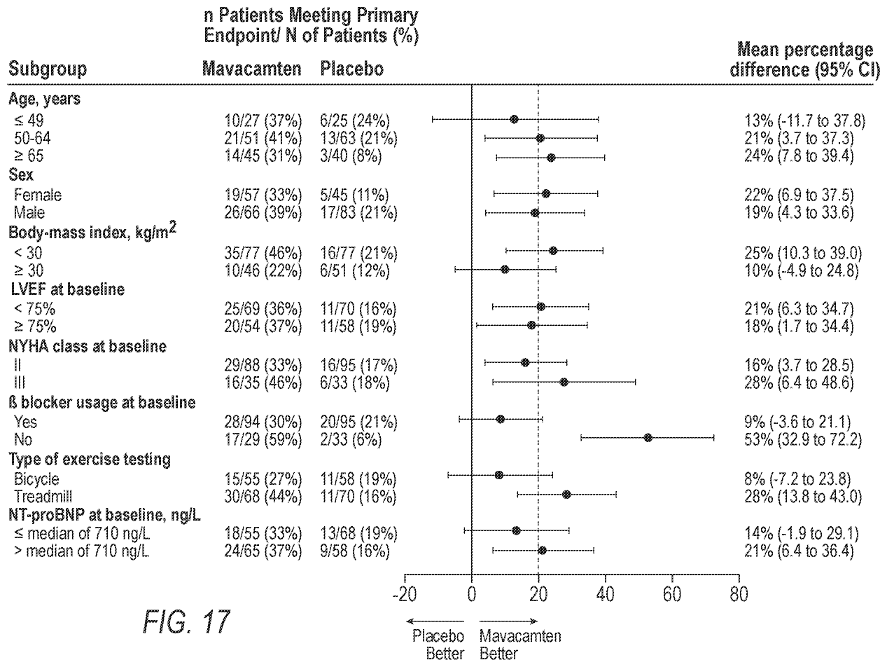

Mavacamten is metabolized primarily by the CYP2C19 enzyme and secondarily by the CYP3A4 enzyme. Concomitant administration of drugs inhibiting CYP2C19 and/or CYP3A4 may therefore affect the metabolism of mavacamten and the resulting exposure of a patient to mavacamten. The present inventors have determined that concomitant administration of mavacamten with a weak CYP2C19 inhibitor or a moderate CYP3A4 inhibitor can be safely achieved under certain conditions. Example 5 and FIG. 17 demonstrate the extent of exposure changes expected with concomitant administration of a weak CYP2C19 inhibitor or a moderate CYP3A4 inhibitor under the conditions described herein. Methods of concomitant administration are described herein below.

Methods described herein comprise administration of a myosin inhibitor. In some embodiments, the myosin inhibitor is mavacamten or a pharmaceutically acceptable salt thereof. In some embodiments, the myosin inhibitor is mavacamten. Mavacamten has the following structure:

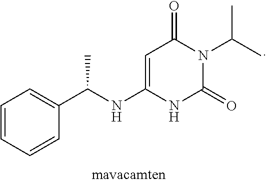 [SMILES: CC(C)n1c(=O)cc(N[C@@H](C)c2ccccc2)[nH]c1=O]

Mavacamten is also known as MYK-461. Its chemical name is (S)-3-Isopropyl-6-((1-phenylethyl)amino)pyrimidine-2,4(1H,3H)-dione or 3-(1-methylethyl)-6-[[(1S)-1-phenylethyl]amino]-2,4(1H,3H)-pyrimidinedione.

Mavacamten can be obtained according to the production methods described in U.S. Pat. No. 9,181,200, which is incorporated herein by reference in its entirety and for all purposes.

In some embodiments, a myosin inhibitor is a compound of formula (I):

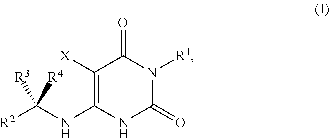

or a pharmaceutically acceptable salt thereof, wherein
R¹ is C₁₋₈ alkyl, C₃₋₈ cycloalkyl, or a phenyl, wherein R¹ is optionally substituted with one or two halo;
R² is phenyl optionally substituted with one or two halo;
R³ is C₁₋₈ alkyl or C₃₋₈ cycloalkyl, wherein each R³ is optionally substituted with halo, hydroxyl or C₁₋₂ alkoxy;
R⁴ is H; and
X is H.

In some embodiments, a myosin inhibitor of formula (I) or a pharmaceutically acceptable salt thereof is selected from group (I) consisting of:

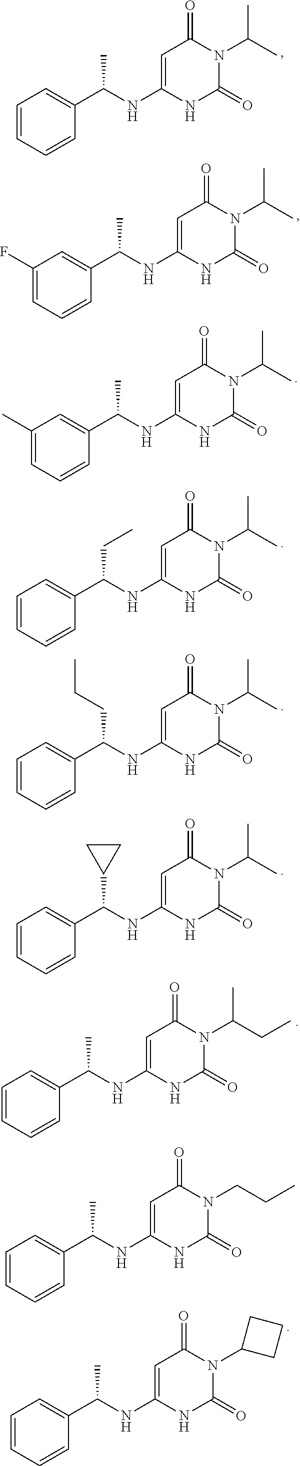

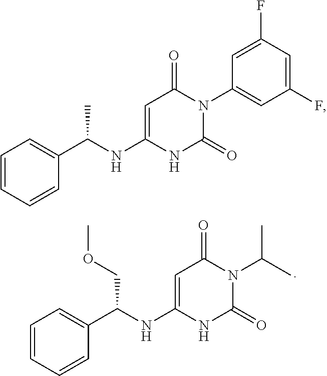 [SMILES: C[C@H](Nc1cc(=O)n(-c2cc(F)cc(F)c2)c(=O)[nH]1)c1ccccc1 ; COC[C@H](Nc1cc(=O)n(C(C)C)c(=O)[nH]1)c1ccccc1]

In some embodiments, a myosin inhibitor of formula (I) is MYK-581 or a pharmaceutically acceptable salt thereof having the following structure:

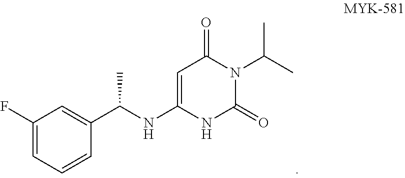 [SMILES: CC(C)n1c(=O)cc(N[C@@H](C)c2cccc(F)c2)[nH]c1=O]

MYK-581's chemical name is (S)-6-((1-(3-fluorophenyl)ethyl)amino)-3-isopropylpyrimidine-2,4(1H,3H)-dione.

Myosin inhibitors of formula (I), including the compounds of group (I), mavacamten, or MYK-581, or a pharmaceutically acceptable salt thereof, can be obtained according to the production methods described in U.S. Pat. No. 9,181,200, which is incorporated herein by reference in its entirety and for all purposes.

In some embodiments, a myosin inhibitor is a compound of formula (II):

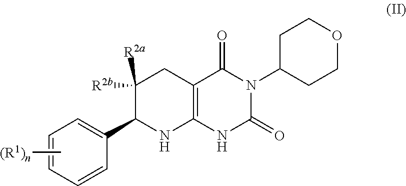

or a pharmaceutically acceptable salt thereof, wherein
R¹ is fluoro, chloro, C₁₋₄ alkyl, C₁₋₄ haloalkyl, C₁₋₄ alkoxy, C₁₋₄ haloalkoxy, or C₂₋₄ alkynyl, wherein at least one R¹ is fluoro; and one of R^{2a} and R^{2b} is fluoro and the other of R^{2a} and R^{2b} is H, and n is 1 or 2.

In some embodiments, a myosin inhibitor of formula (II) or a pharmaceutically acceptable salt thereof is selected from group (II) consisting of:

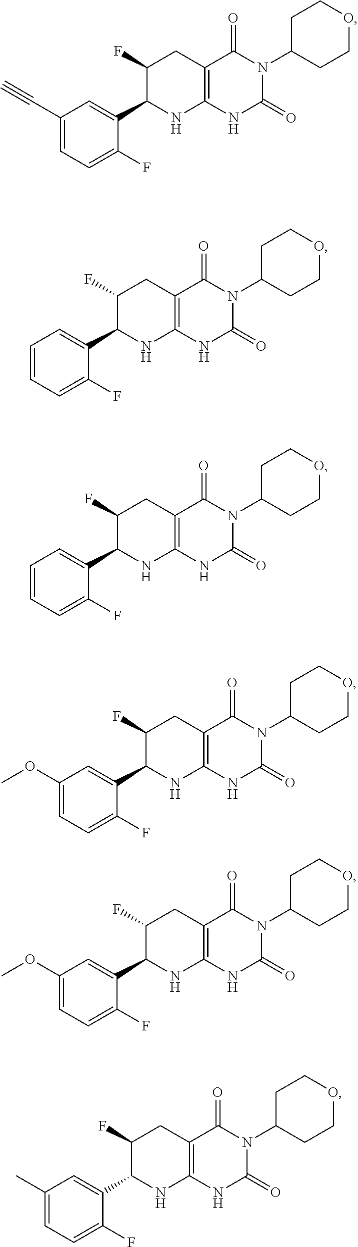 [SMILES: C#Cc1ccc(F)c([C@@H]2Nc3[nH]c(=O)n(C4CCOCC4)c(=O)c3C[C@@H]2F)c1 ; O=c1[nH]c2c(c(=O)n1C1CCOCC1)C[C@@H](F)[C@H](c1ccccc1F)N2 ; O=c1[nH]c2c(c(=O)n1C1CCOCC1)C[C@H](F)[C@H](c1ccccc1F)N2 ; COc1ccc(F)c([C@@H]2Nc3[nH]c(=O)n(C4CCOCC4)c(=O)c3C[C@@H]2F)c1 ; COc1ccc(F)c([C@@H]2Nc3[nH]c(=O)n(C4CCOCC4)c(=O)c3C[C@H]2F)c1 ; Cc1ccc(F)c([C@H]2Nc3[nH]c(=O)n(C4CCOCC4)c(=O)c3C[C@@H]2F)c1]

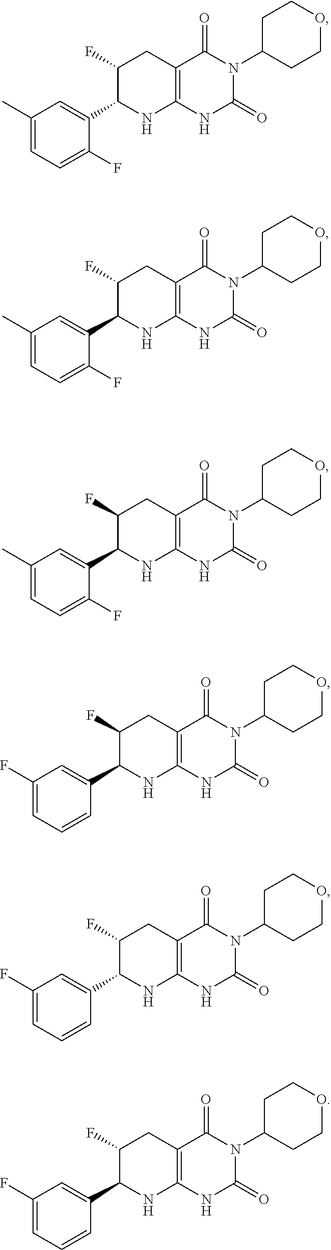 [SMILES: Cc1ccc(F)c([C@H]2Nc3[nH]c(=O)n(C4CCOCC4)c(=O)c3C[C@H]2F)c1 ; Cc1ccc(F)c([C@@H]2Nc3[nH]c(=O)n(C4CCOCC4)c(=O)c3C[C@H]2F)c1 ; Cc1ccc(F)c([C@@H]2Nc3[nH]c(=O)n(C4CCOCC4)c(=O)c3C[C@@H]2F)c1 ; O=c1[nH]c2c(c(=O)n1C1CCOCC1)C[C@H](F)[C@H](c1cccc(F)c1)N2 ; O=c1[nH]c2c(c(=O)n1C1CCOCC1)C[C@@H](F)[C@@H](c1cccc(F)c1)N2 ; O=c1[nH]c2c(c(=O)n1C1CCOCC1)C[C@@H](F)[C@H](c1cccc(F)c1)N2]

Myosin inhibitors of formula (II), including the compounds of group (II), or a pharmaceutically acceptable salt thereof, can be obtained according to the production methods described in International Application Number PCT/US2019/058297, filed on Oct. 29, 2019, which is incorporated herein by reference in its entirety and for all purposes.

In some embodiments, a myosin inhibitor is a compound of formula (III):

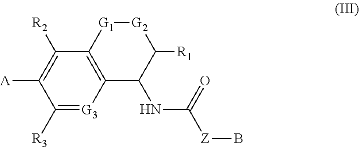

or a pharmaceutically acceptable salt thereof, wherein
G₁ is —CR₄R₅— or —O—;
G₂ is a bond or —CR⁶R⁷—;
G₃ is —CR⁸— or —N—;
R¹, R³, R⁴, R⁵, R⁶, R⁷, and R⁸ are each independently H, C₁-C₆ alkyl, halo, or hydroxyl;
R² is H, C₂-C₆ alkyl, halo, or hydroxyl;
Z is a bond, C₁-C₆ alkyl, —O—, —N(R⁹)—, —R^{X}O—, —OR^{Y}, or —R^{Z}S—;
R⁹ is H, C₁-C₆ alkyl, or cycloalkyl;
A is selected from the group consisting of substituted C₂ alkynyl, unsubstituted C₂ alkynyl, substituted phenyl, unsubstituted phenyl, and 5- or 6-membered heteroaryl comprising at least one annular N atom, wherein the 5- or 6-membered heteroaryl is unsubstituted or substituted with one or more R¹⁰ substituents:
each R¹⁰ is independently substituted or unsubstituted C₁-C₆ alkyl, substituted or unsubstituted C₂-C₆ alkenyl, substituted or unsubstituted C₂-C₆ alkynyl, substituted or unsubstituted cycloalkyl, substituted or unsubstituted heterocycloalkyl, or —C(O)OR^{a};
B is selected from the group consisting of H, C₁-C₆ alkyl, cycloalkyl, aryl, heterocycloalkyl, and heteroaryl, wherein the C₁-C₆ alkyl, cycloalkyl, aryl, heterocycloalkyl, or heteroaryl of B is unsubstituted or substituted with one or more R¹¹ substituents;
each R¹¹ is independently selected from the group consisting of substituted or unsubstituted heterocycloalkyl, substituted or unsubstituted heteroaryl, substituted or unsubstituted cycloalkyl, substituted or unsubstituted aryl, unsubstituted C₁-C₆ alkyl, C₁-C₆ alkyl substituted with one or more R′2 substituents, substituted or unsubstituted C₂-C₆ alkenyl, substituted or unsubstituted C₂-C₆ alkynyl, halo, —OR^{b}, —C(O)R^{c}, — C(O)OR^{d}, oxo, and —NR^{e}R^{f};
each R¹² is independently selected from the group consisting of halo, —OR^{b}, —C(O)R^{g}, —C(O)OR^{h}, and —C(O)NRⁱR^{j};
each R^{a}, R^{b}, R^{c}, R^{d}, R^{e}, R^{f}, R^{g}, R^{h}, Rⁱ, and R^{j} is independently H or C₁-C₆ alkyl; and
R^{X}, R^{Y}, and R^{Z} are each C₁-C₆ alkyl.

In some embodiments, a myosin inhibitor of formula (III) or a pharmaceutically acceptable salt thereof is selected from group (III) consisting of:

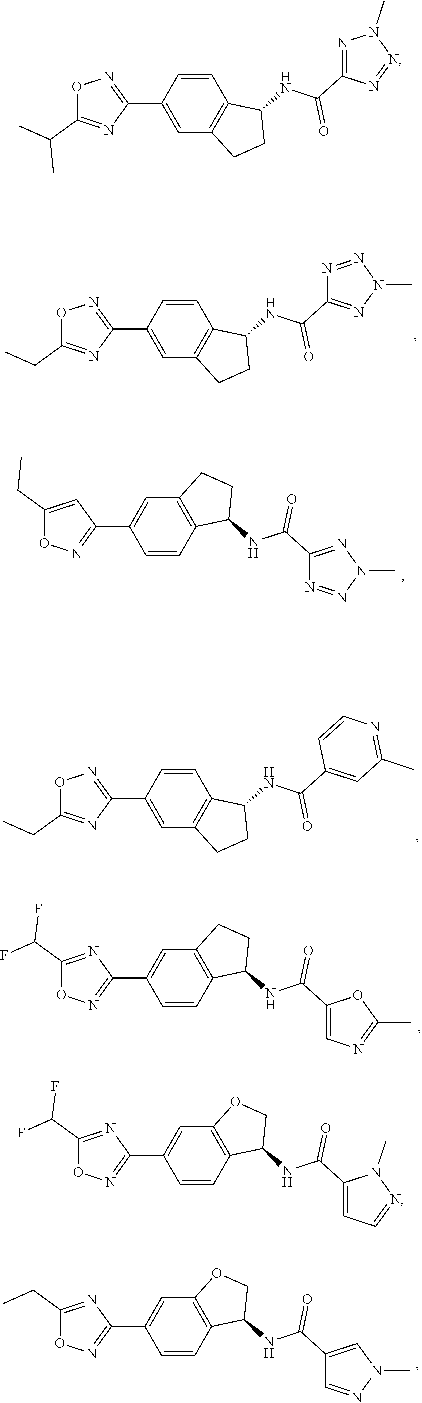

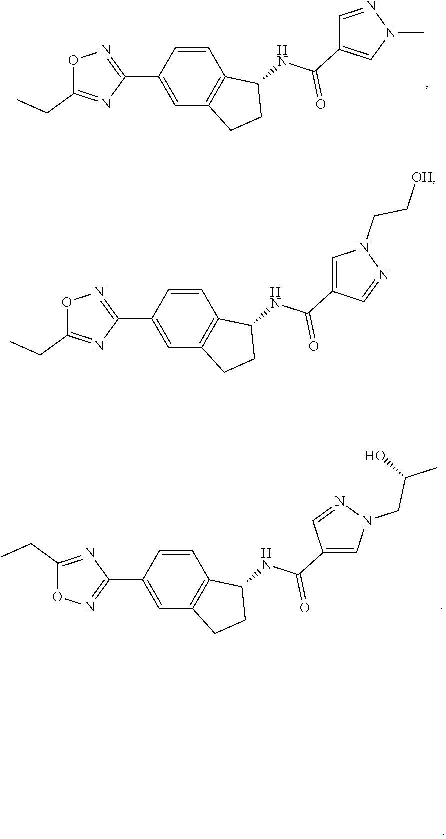

Myosin inhibitors of formula (III), including the compounds of group (III), or a pharmaceutically acceptable salt thereof, can be obtained according to the production methods described in International Publication Number WO 2019/144041, published on Jul. 25, 2019, which is incorporated herein by reference in its entirety and for all purposes.

In some embodiments, the myosin inhibitor is aficamten or a pharmaceutically acceptable salt thereof. Aficamten has the following structure:

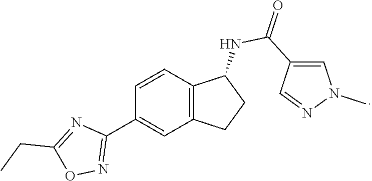 [SMILES: CCc1nc(-c2ccc3c(c2)CC[C@H]3NC(=O)c2cnn(C)c2)no1]

In some embodiments, myosin inhibitors include the compounds disclosed in PCT patent applications, published as WO2020/005887, WO2020/005888, WO2020/047447, which are incorporated herein by reference in its entirety and for all purposes.

The myosin inhibitors of the present invention are generally administered in a pharmaceutical composition. The pharmaceutical compositions for the administration of a compound of formulas (I), (II), (III), and/or a compound of groups (I), (II), (III), and/or mavacamten, and/or MYK-581 or a pharmaceutically acceptable salt thereof may conveniently be presented in unit dosage form and may be prepared by any of the methods known in the art of pharmacy and drug delivery. Such methods include the step of bringing the active ingredient into association with a carrier containing one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active agent is generally included in an amount sufficient to produce the desired effect upon myocardial contractility (i.e. to decrease the often supranormal systolic contractility in HCM) and to improve left ventricular relaxation in diastole. Such improved relaxation can alleviate symptoms in hypertrophic cardiomyopathy and other etiologies of diastolic dysfunction. It can also ameliorate the effects of diastolic dysfunction causing impairment of coronary blood flow, improving the latter as an adjunctive agent in angina pectoris and ischemic heart disease. It can also confer benefits on adverse left ventricular remodeling in HCM and other causes of left ventricular hypertrophy due to chronic volume or pressure overload from, e.g., valvular heart disease or systemic hypertension.

The pharmaceutical compositions containing a compound of formulas (I), (II), (III), and/or a compound of groups (I), (II), (III), and/or mavacamten, and/or MYK-581 or a pharmaceutically acceptable salt thereof, may be in a form suitable for oral use, for example, as tablets, troches, lozenges, aqueous or oily suspensions, dispersible powders or granules, emulsions, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

In some embodiments, a compound of formulas (I), (II), (III), and/or a compound of groups (I), (II), (III), and/or mavacamten, and/or MYK-581 can be used in the form of a pharmaceutically acceptable salt. Examples of the pharmaceutically acceptable salt include salts with inorganic bases, salts with organic bases, salts with inorganic acids, salts with organic acids, and salts with basic or acidic amino acids. In other embodiments, a compound of formulas (I), (II), (III), and/or a compound of groups (I), (II), (III), and/or mavacamten, and/or MYK-581 can be used in the form of a free base.

The present disclosure includes novel pharmaceutical dosage forms of mavacamten or a pharmaceutically acceptable salt thereof. The dosage forms described herein are suitable for oral administration to a patient. The dosage form may be in any form suitable for oral administration, including, but not limited to, a capsule or a tablet. In some embodiments, the present disclosure provides a single unit dosage capsule or tablet form containing 1-25 mg (e.g., 1, 1.5, 2, 2.5, 3, 3.5, 4, 4.5, 5, 6, 7, 7.5, 8, 9, 10, 11, 12, 12.5, 13, 14, 15, 16, 17, 18, 19, 20, 21, 22, 23, 24, or 25 mg) of mavacamten or a pharmaceutically acceptable salt thereof. In some embodiments, the amount of mavacamten in a unit dosage is from about 2 to 5 mg, from about 5 to 10 mg, about 2.5 mg or about 5 mg. In some embodiments, the single unit dosage form is a capsule. In some embodiments, the single unit dosage form is a tablet. In some embodiments, the pharmaceutical composition is a capsule filled with mavacamten, silicon dioxide, mannitol, hypromellose, croscarmellose sodium, and magnesium stearate. In some embodiments, the capsule shell contains gelatin, and optionally titanium dioxide, black iron oxide, red iron oxide and/or yellow iron oxide.

Methods of safely administering myosin inhibitors are described herein. Various aspects of these methods include adjusting the dose of a myosin inhibitor administered to a patient over time based on assessments of the patient over time, particularly assessments of left ventricular outflow tract obstruction and/or left ventricular ejection fraction; methods comprising discontinuing administration, where certain conditions are met; methods of administering a myosin inhibitor to a patient where the patient receives concomitant administration of certain other drug(s); methods of administering a myosin inhibitor to avoid harmful drug-drug interactions; and methods of controlling the distribution of a myosin inhibitor to mitigate risk.

Myosin inhibitors may be administered by suitable means as known and described in the art. In some embodiments, the myosin inhibitors are administered orally. In some embodiments, the myosin inhibitors are administered in a pharmaceutical composition. In some embodiments, the pharmaceutical composition is an oral dosage form.

Examples of oral dosage forms include tablets, troches, lozenges, aqueous or oily suspensions, dispersible powders or granules, emulsions, hard or soft capsules, syrups, elixirs, solutions, buccal patch, oral gel, chewing gum, chewable tablets, effervescent powder and effervescent tablets. In some embodiments, the oral dosage form is a capsule. In other embodiments, the oral dosage form is a tablet.

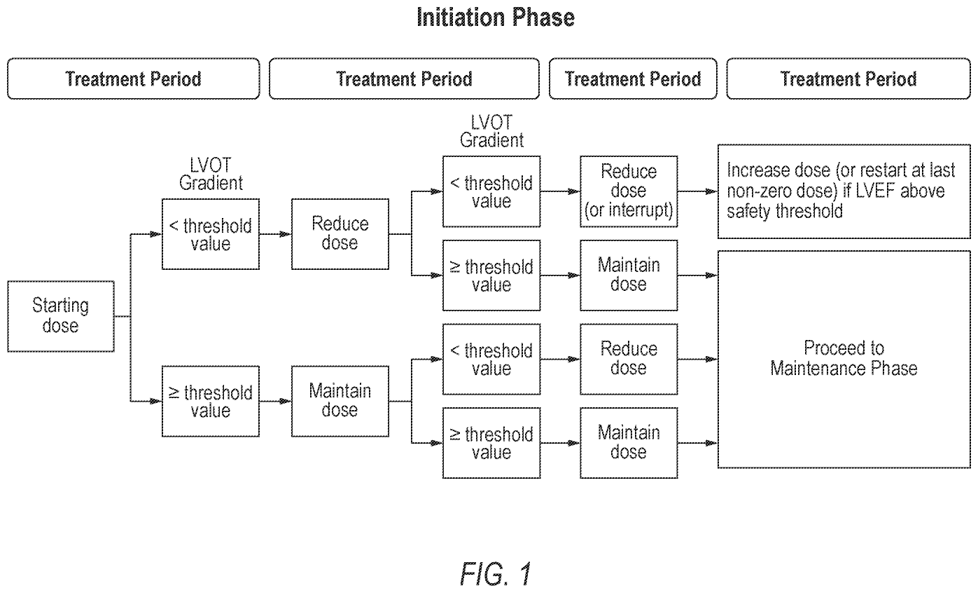

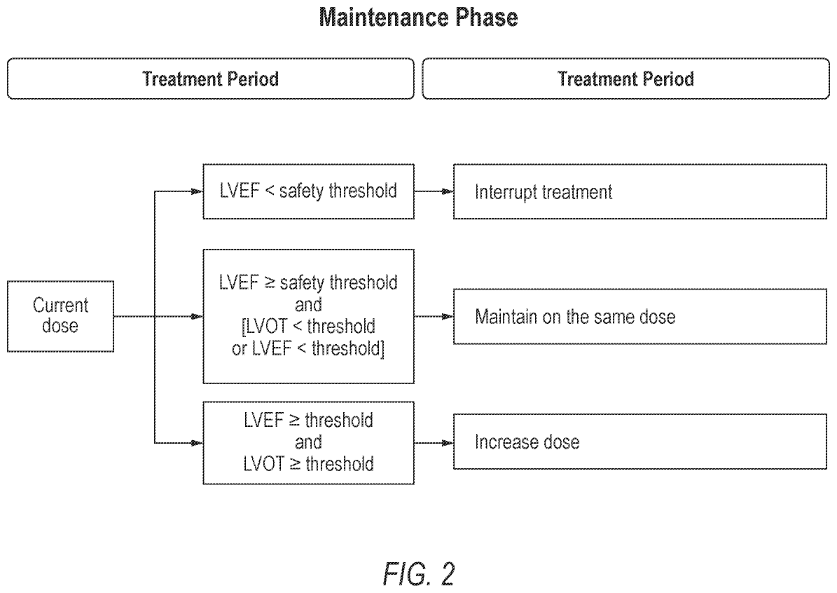

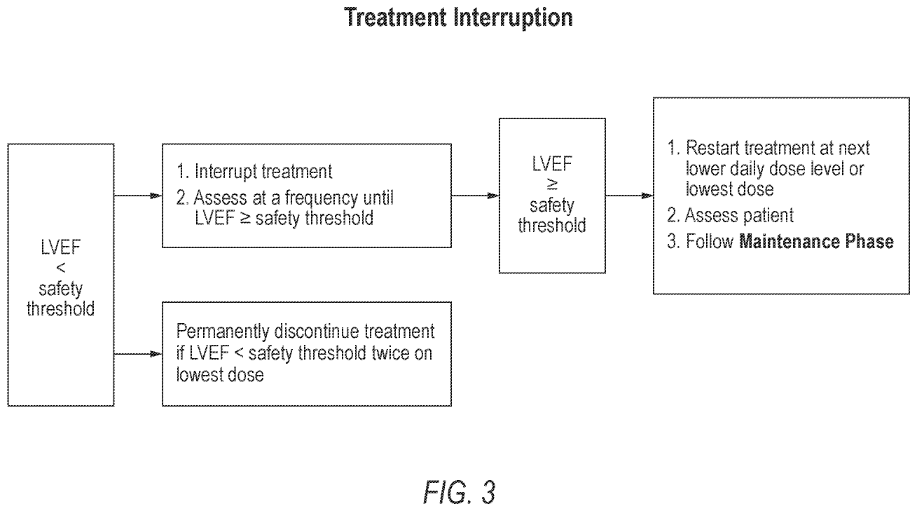

According to the present disclosure, the dosage of the myosin inhibitor may be adjusted over time, i.e., by increasing the dose, decreasing the dose, maintaining the dose, or interrupting dosing (temporary discontinuation). In some embodiments, the dose is adjusted step-wise. FIG. 1 shows an initiation phase for a dosing scheme for a myosin inhibitor during which dose may be adjusted from one treatment period to the next. During the initiation phase, a starting dose is administered, and then is maintained or reduced. FIG. 2 shows a maintenance phase during which dose may be adjusted by interrupting treatment, maintaining the dose, or increasing the dose. FIG. 3 shows a treatment interruption aspect of the dosing scheme, whereby treatment may be interrupted and then restarted at a lower dose. For example, a daily dose of mavacamten may be adjusted between some or all of the following daily doses: 0 mg, 2.5 mg, 5 mg, 10 mg, and 15 mg. For example, a dose of 5 mg QD may be administered and then either increased to 10 mg QD, decreased to 2.5 mg QD, or maintained at 5 mg QD. In some embodiments, the step-wise dosing may include 1 mg as an additional dose. In some embodiments, the step-wise dosing may include 7.5 mg as an additional dose. 7.5 mg may be included as an additional dose level in the embodiments described herein; embodiments describing an increase from 5 to 10 mg QD may alternatively use an increase from 5 to 7.5 mg QD, and further increases from 7.5 to 10 mg QD and 10 mg to 15 mg QD are possible during subsequent treatment periods based on the outcomes of the relevant assessments.

In some embodiments, the dose is a total daily dose administered once daily (QD). In other embodiments, the dose is a total daily dose administered in two separate administrations per day (twice daily, B.I.D.).

The present disclosure relates to methods of safely administering a myosin inhibitor to a patient. Various aspects and embodiments of such methods are described herein. In some embodiments, the methods include a plurality of treatment periods during which the myosin inhibitor is administered to the patient, or optionally during which administration is temporarily discontinued. An assessment may be performed at or near the conclusion of a treatment period, and the outcome of the assessment may be used to determine whether the dose administered during the treatment period should be increased, maintained, decreased, or discontinued during the subsequent treatment period. The assessments and corresponding dose adjustments provide for safe and effective administration of the myosin inhibitor.

Assessments are described in further detail below but may comprise an assessment of left ventricular outflow tract obstruction (e.g., a Valsalva LVOT gradient) and/or an assessment of left ventricular ejection fraction (LVEF). The assessment may take place during a treatment period, or at or near the conclusion of a treatment period, for example during the final week of a multi-week treatment period. The dosing of the myosin inhibitor may be adjusted from one treatment period to the next treatment period based on the assessment outcome. In some embodiments, a treatment period (e.g., a first treatment period), is immediately followed by the next treatment period (e.g., a second treatment period). For example, a first treatment period from days 1-28 may be immediately followed by a second treatment period from days 29-56 where the myosin inhibitor is administered once daily for each of days 1-56. Where there are a plurality of treatment periods, each treatment period may directly follow the prior treatment period, whereby the myosin inhibitor is administered without interruption from one treatment period to the next. In other embodiments, administration of the myosin inhibitor may be interrupted in between treatment periods.

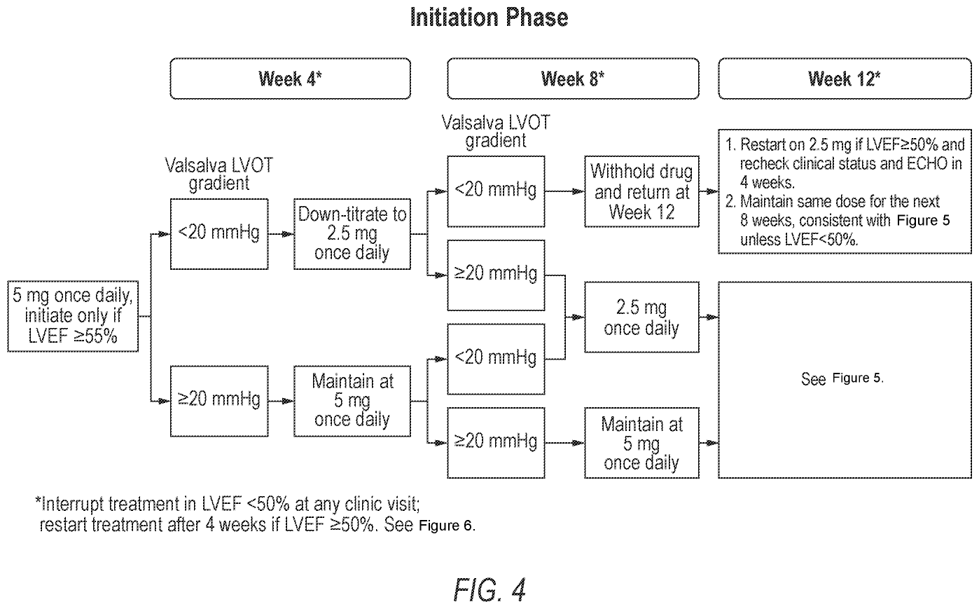

In some embodiments, a starting dose of a myosin inhibitor is administered during a first treatment period. Referring to FIG. 1, a starting dose is administered during a first treatment period and an assessment is made at or near the conclusion of the first treatment period, which determines the dose administered during the second treatment period. In some embodiments, the first treatment period is part of an initiation phase, during which the dose of the myosin inhibitor is not increased, and during which the dose of the myosin inhibitor may be decreased. As shown in FIG. 1, the dose may be reduced or maintained from the first to the second treatment period, and from the second to the third treatment period, but may not be increased. For example, as shown in FIG. 4, the initiation phase for mavacamten may have a starting dose of 5 mg QD of mavacamten. In some embodiments, the first treatment period is from about 1 to about 12 weeks in duration. In some embodiments, the first treatment period is from about 2 to about 12 weeks in duration. In some embodiments, the first treatment period is from about 2 to about 8 weeks in duration. In some embodiments, the first treatment period is from about 2 to about 6 weeks in duration. In some embodiments, the first treatment period is from about 3 to about 4 weeks in duration. In some embodiments, the first treatment period is about 4 weeks in duration. In some embodiments, the first treatment period is from about 20 to about 35 days in duration. In some embodiments, the first treatment period is from about 22 to about 28 days in duration.

In some embodiments, a second dose of a myosin inhibitor is administered during a second treatment period (i.e., following the first treatment period). As shown in FIG. 1, the second dose is determined based on the first assessment outcome and is administered during the second treatment period. An assessment is also made at or near the conclusion of the second treatment period, which determines the dose administered during the third treatment period. In some embodiments, the second treatment period is part of an initiation phase, during which the dose of the myosin inhibitor is not increased, and during which the dose of the myosin inhibitor may be decreased. In some embodiments, the second dose is less than or equal to the starting dose. In some embodiments the second dose is less than the starting dose. In some embodiments, the second dose is the same as the starting dose. For example, as shown in FIG. 4, the second dose of mavacamten may be 2.5 mg QD of mavacamten. As another example, as shown in FIG. 4, the second dose of mavacamten may be 5 mg QD of mavacamten. In some embodiments, the second treatment period is from about 1 to about 12 weeks in duration. In some embodiments, the second treatment period is from about 2 to about 12 weeks in duration. In some embodiments, the second treatment period is from about 2 to about 8 weeks in duration. In some embodiments, the second treatment period is from about 2 to about 6 weeks in duration. In some embodiments, the second treatment period is from about 3 to about 4 weeks in duration. In some embodiments, the second treatment period is about 4 weeks in duration. In some embodiments, the second treatment period is from about 20 to about 35 days in duration. In some embodiments, the second treatment period is from about 22 to about 28 days in duration.

In some embodiments, a third dose of a myosin inhibitor is administered during a third treatment period (i.e., following the second treatment period). As shown in FIG. 1, the third dose is determined based on the second assessment outcome and is administered during the third treatment period. An assessment is also made at or near the conclusion of the third treatment period, which determines the dose administered during the fourth treatment period. In some embodiments, the third treatment period is part of an initiation phase, during which the dose of the myosin inhibitor is not increased, and during which the dose of the myosin inhibitor may be decreased. In some embodiments, the third dose is less than or equal to the starting dose. In some embodiments, the third dose is less than the starting dose. In some embodiments, the third dose is the same as the starting dose. In some embodiments, the third dose is less than or equal to the second dose. In some embodiments, the third dose is less than the second dose. In some embodiments, the third dose is the same as the second dose. In some embodiments, the third dose is 2.5 mg QD of mavacamten. For example, the third dose of mavacamten may be 5 mg QD of mavacamten. As another example, the third dose may be 1 mg QD of mavacamten. In some instances, the third dose of mavacamten is 0 mg QD of mavacamten. FIG. 4 shows an example of the different doses that may be administered as the third dose during the third treatment period. Administration of 0 mg of a myosin inhibitor (e.g., mavacamten) during a treatment period refers to a period during which the myosin inhibitor (e.g., mavacamten) is withheld from the patient. In some embodiments, the third dose is 0 mg QD or 1 mg QD of mavacamten, and the patient is a poor metabolizer. In some embodiments, the third dose is 0 mg QD or 1 mg QD of mavacamten, and the patient receives concomitant administration of a CYP2C19 inhibitor or inducer (e.g., a weak CYP2C19 inhibitor or inducer), or a CYP3A4 inhibitor or inducer (e.g., a weak or moderate CYP3A4 inhibitor or inducer). In some embodiments, no myosin inhibitor is administered during the third treatment period. In some embodiments, the third treatment period is from about 1 to about 12 weeks in duration. In some embodiments, the third treatment period is from about 2 to about 12 weeks in duration. In some embodiments, the third treatment period is from about 2 to about 8 weeks in duration. In some embodiments, the third treatment period is from about 2 to about 6 weeks in duration. In some embodiments, the third treatment period is from about 3 to about 4 weeks in duration. In some embodiments, the third treatment period is about 4 weeks in duration. In some embodiments, the third treatment period is from about 20 to about 35 days in duration. In some embodiments, the third treatment period is from about 22 to about 28 days in duration.

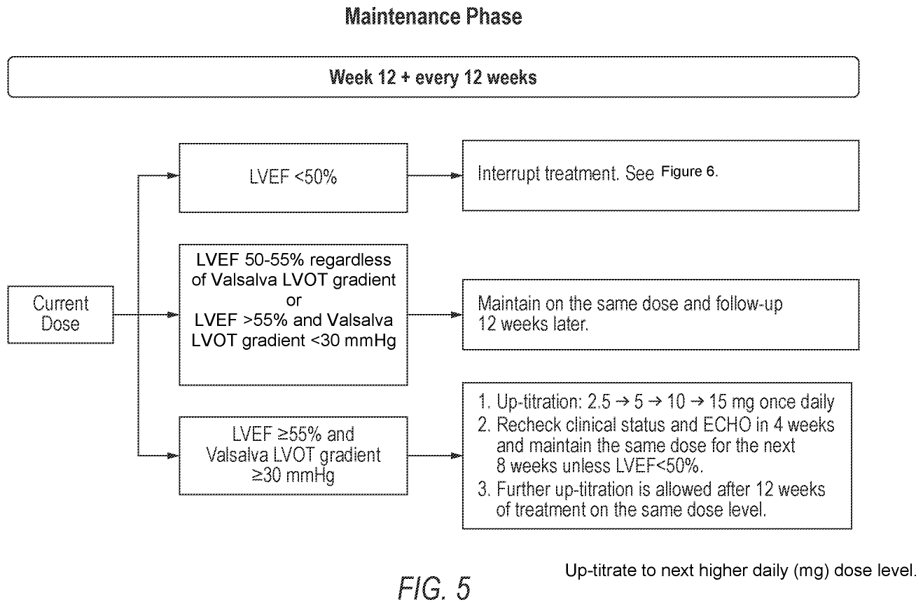

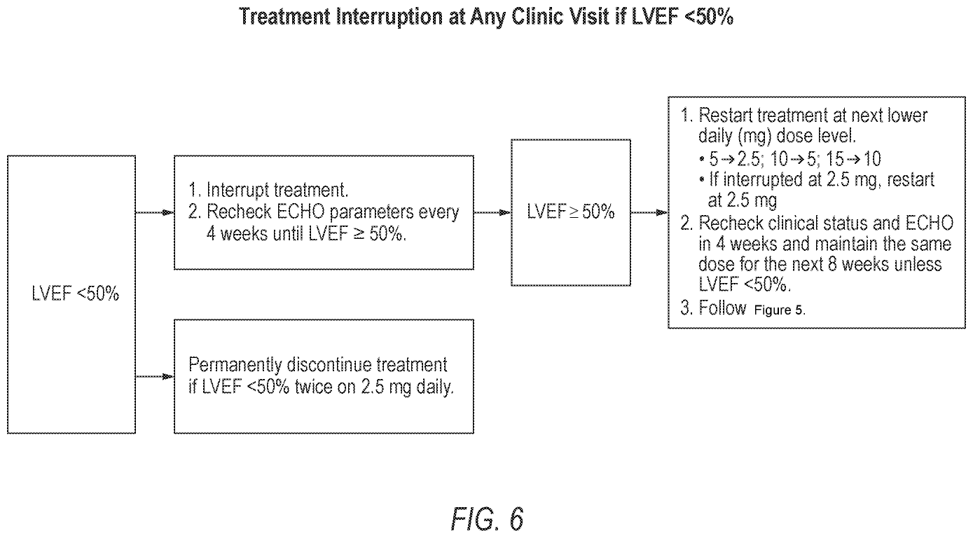

In some embodiments, a fourth dose of a myosin inhibitor is administered during a fourth treatment period (i.e., following the third treatment period). As shown in FIG. 1, the fourth treatment period may be considered the beginning of a maintenance phase, following the initiation phase. As shown in FIG. 2, the maintenance phase utilizes criteria for increasing dose, maintaining the same dose, or interrupting treatment (temporary discontinuation) based on an assessment outcome (e.g., of LVEF and LVOT gradient). In some embodiments, the fourth treatment period is part of a maintenance phase, during which the dose of the myosin inhibitor may be increased. In some embodiments, the fourth dose is greater than the third dose. In some embodiments, where the fourth dose is greater than the third dose, this represents the first dose increase during the treatment. Thus, the fourth dose may be the first increased dose and the fourth treatment period may be the first treatment period during which an increased dose is administered. In some embodiments, the fourth dose is the same as the third dose. In some embodiments, the fourth dose is 0 mg, 1 mg, 2.5 mg, 5 mg, or 10 mg QD of mavacamten. FIG. 5 shows an example of criteria for determining the fourth dose. In some embodiments, the fourth treatment period is from about 2 to about 26 weeks (or 6 months) in duration. In some embodiments, the fourth treatment period is from about 4 to about 24 weeks in duration. In some embodiments, the fourth treatment period is from about 4 to about 16 weeks in duration. In some embodiments, the fourth treatment period is from about 8 to about 24 weeks in duration. In some embodiments, the fourth treatment period is from about 8 to about 16 weeks in duration. In some embodiments, the fourth treatment period is about 12 weeks (or about 3 months) in duration. In some embodiments, the fourth treatment period is from about 80 to about 100 days in duration. In some embodiments, the fourth treatment period is from about 84 to about 90 days in duration.

In some embodiments, a fifth dose of a myosin inhibitor is administered during a fifth treatment period (i.e., following the fourth treatment period). In some embodiments, the fifth treatment period is part of a maintenance phase, during which the dose of the myosin inhibitor may be increased. The fifth dose may be determined in accordance with FIG. 2. In some embodiments, the fifth dose is greater than the fourth dose. In some embodiments, the fifth dose is the same as the fourth dose. In some embodiments, the fifth dose is 2.5 mg, 5 mg, 10 mg, or 15 mg QD of mavacamten. FIG. 5 shows an example of criteria for determining the fifth dose during a maintenance phase. In some embodiments, the fifth treatment period is from about 2 to about 26 weeks (or 6 months) in duration. In some embodiments, the fifth treatment period is from about 4 to about 24 weeks in duration. In some embodiments, the fifth treatment period is from about 4 to about 16 weeks in duration. In some embodiments, the fifth treatment period is from about 8 to about 24 weeks in duration. In some embodiments, the fifth treatment period is from about 8 to about 16 weeks in duration. In some embodiments, the fifth treatment period is about 12 weeks (or about 3 months) in duration. In some embodiments, the fifth treatment period is from about 80 to about 100 days in duration. In some embodiments, the fifth treatment period is from about 84 to about 90 days in duration.

In some embodiments, the method further comprises additional treatment periods following the fifth treatment period (i.e., a sixth treatment period, a seventh treatment period, etc.) These additional treatment periods may be part of the maintenance phase, during which the dose of the myosin inhibitor may be increased. In some embodiments, the dose administered during additional treatment periods is determined in accordance with FIG. 2. In some embodiments, the additional treatment periods are equal to the fourth and/or fifth treatment periods in duration.

In some embodiments, administration of the myosin inhibitor is temporarily discontinued, e.g., during the first, second, third, fourth, or fifth treatment period. As shown in FIG. 3, temporary discontinuation (treatment interruption), may be indicated based on an assessment outcome, e.g., based on LVEF. In some embodiments, the temporary discontinuation is from about 1 to about 12 weeks in duration, e.g., about 2-8 weeks, about 3-4 weeks, or about 4 weeks in duration. In some embodiments, temporary discontinuation is triggered by the patient having a LVEF below a safety threshold, e.g., as described herein. In some embodiments, temporary discontinuation is triggered by the patient having a LVOT gradient below a threshold, e.g., as described herein. For example, as shown in FIG. 6, the threshold may be 50%. Thus, during treatment with the myosin inhibitor for a plurality of treatment periods, administration of the myosin inhibitor may be temporarily discontinued when an assessment during a treatment period shows that LVEF and/or LVOT gradient is below a threshold; and the temporary discontinuation may be implemented by not administering the myosin inhibitor during the treatment period immediately following the treatment period during which the assessment was performed.

In some embodiments, administration of the myosin inhibitor is permanently discontinued. For example, permanent discontinuation may be implemented after the second, third, fourth, or fifth treatment period. In some embodiments, administration is permanently discontinued after treatment was temporarily discontinued during a previous treatment period. In some embodiments, administration is permanently discontinued after treatment was temporarily discontinued during a previous treatment period and resumed following the temporary discontinuation. As shown in FIG. 3, administration may be permanently discontinued if LVEF is less than a safety threshold for a second time while receiving the lowest dose (both times). Permanent discontinuation may be determined based on an assessment, e.g., an LVEF assessment with an outcome that LVEF is below a safety threshold. In particular, in some embodiments, administration is permanently discontinued when LVEF is less than 50% at an assessment that occurs after resuming administration, when administration had been previously discontinued due to LVEF less than 50%. For example, as shown in FIG. 6, administration is permanently discontinued when LVEF is less than 50% at an assessment that occurs after resuming administration at 2.5 mg QD of mavacamten, when administration had been previously discontinued due to LVEF less than 50% during administration of 2.5 mg QD of mavacamten. In some embodiments, treatment may be resumed, despite the criteria for permanent discontinuation being met, if the discontinuation was due to transient factors.

Certain methods described herein include an initiation phase. In some embodiments, the initiation phase comprises the first and second treatment periods. In some embodiments, the initiation phase comprises the first, second, and third treatment periods. In some embodiments, the initiation phase comprises the first, second, third, and fourth treatment periods. In some embodiments, the initiation phase comprises the first, second, and third treatment periods, and a portion of the fourth treatment period. In some embodiments, the dose of the myosin inhibitor is not increased during the initiation phase. In some embodiments, the myosin inhibitor does not reach steady state pharmacokinetics during the initiation phase, or does not reach steady state pharmacokinetics until at or near the conclusion of the initiation phase in certain patients (e.g., poor metabolizers). In some embodiments, the myosin inhibitor is mavacamten and reaches steady state pharmacokinetics in the patient after about 10 weeks of daily administration. In some embodiments, the myosin inhibitor is mavacamten and reaches steady state pharmacokinetics in certain patients (e.g., poor metabolizers) after about 12 weeks of daily administration.

Certain methods described herein include a maintenance phase. In some embodiments, the maintenance phase comprises the fourth treatment period. In some embodiments, the maintenance phase comprises the fifth treatment period. In some embodiments, the maintenance phase comprises additional treatment periods subsequent to the fourth and fifth treatment periods. In some embodiments, the myosin inhibitor is at a steady state during the maintenance phase, or reaches a steady state at or near the beginning of the maintenance phase.

In some embodiments, a myosin inhibitor is administered concomitantly with a weak CYP2C19 inhibitor or moderate CYP3A4 inhibitor. In some embodiments, a myosin inhibitor is administered concomitantly with a weak CYP2D6 inhibitor (e.g., aficamten). Additional steps may be required to provide safe administration of a myosin inhibitor when administered concomitantly with such agents. As described above, mavacamten is primarily metabolized by CYP2C19 and secondarily metabolized by CYP3A4. Thus, agents inhibiting these enzymes can lead to reduced metabolism of mavacamten and potentially high exposure, especially in patients who have a poor metabolizer genotype. In some embodiments, the concomitant administration of a weak CYP2C19 inhibitor or moderate CYP3A4 inhibitor is initiated when the patient is receiving myosin inhibitor therapy. In some embodiments, the concomitant administration is initiated when the patient is receiving a stable dose of the myosin inhibitor, e.g., during or after the third, fourth, or fifth treatment period. In some embodiments, the weak CYP2C19 inhibitor or moderate CYP3A4 inhibitor is selected from the group consisting of cimetidine, ciprofloxacin, diltiazem, felbamate, omeprazole at a dose of 20 mg once daily, isoniazid, fluconazole, and verapamil.

Disclosed herein are certain methods for safely concomitantly administering a myosin inhibitor with a weak CYP2C19 inhibitor or moderate CYP3A4 inhibitor. In some embodiments, a first daily dose of the myosin inhibitor is administered during a first treatment period prior to initiating the concomitant therapy with a weak CYP2C19 inhibitor or a moderate CYP3A4 inhibitor; and a second daily dose of the myosin inhibitor, which is less than the first daily dose, is administered during a second treatment period, wherein the patient receives the concomitant therapy with a weak CYP2C19 inhibitor or a moderate CYP3A4 inhibitor during the second treatment period. In some embodiments, disclosed is a method of treating HCM in a patient being administered a first daily dose of mavacamten, wherein said patient is then in need of being treated concurrently with a weak CYP2C19 inhibitor or a moderate CYP3A4 inhibitor in addition to the mavacamten, comprising administering to the patient a second daily dose of mavacamten, which is less than the first daily dose, in addition to administration of the weak CYP2C19 inhibitor or moderate CYP3A4 inhibitor. In some embodiments, the myosin inhibitor is mavacamten the first daily dose is 5 mg, 10 mg, or 15 mg QD of mavacamten, and the second daily dose is 2.5 mg, 5 mg, or 10 mg QD of mavacamten.

When a patient is on a stable therapy with a weak CYP2C19 inhibitor or a moderate CYP3A4 inhibitor, administration of mavacamten may commence at a starting dose of 5 mg. Disclosed herein is a method of administering mavacamten to a patient receiving administration of a weak CYP2C19 inhibitor or a moderate CYP3A4 inhibitor, the method comprising: (a) determining that the patient is on a stable therapy with a weak CYP2C19 inhibitor or a moderate CYP3A4 inhibitor; and (b) initiating administration of mavacamten to the patient at a daily dose of 5 mg per day.

Also disclosed herein is a method of administering a myosin inhibitor (e.g., mavacamten) to a patient who initiates or increases the dose of a concomitant therapy with a negative inotrope while receiving myosin inhibitor therapy, the method comprising: (a) administering a therapeutically effective amount of a myosin inhibitor during a first treatment period; (b) continuing to administer the myosin inhibitor, during a second treatment period, wherein the patient initiates or increases the dose of a concomitant therapy with a negative inotrope during the second treatment period; and (c) providing echocardiographic monitoring of LVEF during the second treatment period. In some embodiments, echocardiographic monitoring of LVEF is provided until stable doses and clinical response have been achieved. In some embodiments, close medical supervision is provided during the second treatment period.

Example 4 describes a study using drug-drug interaction simulation based upon which mavacamten is proposed to be contraindicated with both strong and moderate inducers of CYP2C19 and CYP3A4.

Also disclosed herein is a method of treating obstructive hypertrophic cardiomyopathy (oHCM) in a patient in need thereof, the method comprising administering a therapeutically effective amount of mavacamten to the subject, wherein the patient does not receive concomitant administration of a strong or moderate CYP2C19 inducer nor a strong or moderate CYP3A4 inducer.

Also disclosed herein is a method of treating obstructive hypertrophic cardiomyopathy (oHCM) in a patient in need thereof, where the patient is being treated with a strong or moderate CYP2C19 inducer or a strong or moderate CYP3A4 inducer, the method comprising: discontinuing administration to the patient of the strong or moderate CYP2C19 inducer or strong or moderate CYP3A4 inducer; and administering a therapeutically effective amount of mavacamten to the subject, thereby avoiding the use of mavacamten in combination with a strong or moderate CYP2C19 inducer or a strong or moderate CYP3A4 inducer.

In some embodiments, methods of the present disclosure include one or more assessments of the patient. An exemplary schematic of assessments and corresponding dose adjustments is shown in FIGS. 1-3. The one or more assessments can be used to determine what dose of the myosin inhibitor to administer to the patient. For example, assessment(s) during or at the conclusion of a treatment period can be used to determine the dose of the myosin inhibitor to administer during the next treatment period, e.g., to increase, maintain, reduce, or discontinue the dose during the next treatment period. For example, FIG. 1 shows assessments of LVOT gradient performed or obtained at the conclusion of treatment periods and used to guide the dose administered for the subsequent treatment period. In some embodiments, assessment(s) are performed at or near the conclusion of a treatment period, e.g., during the last week of a multi-week treatment period.

In some embodiments, the patient is assessed for left ventricular outflow tract obstruction. In some embodiments, the patient is assessed for LVOT gradient. In some embodiments, the pressure gradient across the LVOT is measured at rest. In some embodiments, the pressure gradient across the LVOT in the individual is measured during or immediately after a Valsalva maneuver is performed (a “Valsalva LVOT gradient”). In some embodiments, the pressure gradient across the LVOT in the individual is measured post-exercise. In some embodiments, the patient is assessed for LVEF.

In some embodiments, the assessment is performed using a non-invasive technique. In some embodiments, the non-invasive technique is an imaging technique (e.g., cardiac imaging technique). In some embodiments, the non-invasive technique is echocardiography. In some embodiments, the non-invasive technique is two-dimensional echocardiography. In some embodiments, the two-dimensional echocardiography is used to determine LVEF. In some embodiments, the non-invasive technique is Doppler echocardiography. In some embodiments, Doppler echocardiography is used to determine LVOT gradient. In some embodiments, the non-invasive technique is trans-thoracic echocardiography. In alternative embodiments, the non-invasive technique is trans-esophageal cardiography. Cardiac imaging techniques may be utilized as a non-invasive technique. For example, cardiac magnetic resonance imaging may be used. Cardiac magnetic resonance imaging may be used to measure LVEF. An alternative technique for measuring LVEF and/or LVOT gradient is cardiac catheterization.

In some embodiments, the patient is assessed for left ventricular outflow tract obstruction (e.g., LVOT gradient) at the conclusion of each of two or more treatment periods, e.g., during an initiation phase, e.g., as shown in FIG. 1. Based on these assessments, the dose administered to the patient may be reduced to mitigate the risk of systolic dysfunction and heart failure. The multiple (i.e., two or more) assessments of left ventricular outflow tract obstruction, and corresponding dose adjustments, have several benefits. First, by providing more than one assessment that can result in dose reduction during an initiation phase, patients can be identified at second (or subsequent) assessments who will benefit from dose reduction, who were not identified at the first assessment (e.g., due to dangerous exposure levels not being found at the first assessment), thereby mitigating risk of adverse events for those patients. At the same time, a higher starting dose can be used because there are multiple opportunities to reduce the dose based on the assessment of left ventricular outflow tract obstruction, thus making starting at a higher dose safe across the patient population. This benefits a large part of the patient population at lower risk for adverse events by allowing them to receive a more therapeutic dose during the initiation phase. Also, by providing more than one assessment, there is not a need for overly conservative dose reductions following the first assessment since the second (or subsequent) assessments can identify additional patients that will benefit from a dose reduction. This results in more patients who are able to stay on the starting dose throughout the initiation phase, rather than decreasing their dose and then increasing it at a later time. Additionally, by providing more than one assessment that can result in dose reduction during an initiation phase, patients for whom a single dose reduction still results in too much drug exposure can have their dose reduced even lower, or be temporarily taken off drug. Example 2, describes an exemplary dosing scheme for mavacamten and provides data showing the benefit of providing two assessments of LVOT gradient during an initiation phase and allowing for corresponding dose reductions.

In some embodiments, the patient is assessed for both LVOT gradient and LVEF during a plurality of treatment periods. Using LVEF assessments in addition to LVOT gradient assessments may provide further risk mitigation. In some embodiments, the dose administered during a treatment period is determined based on the assessment of LVOT gradient and LVEF during the prior treatment period (e.g., during the final week of the prior treatment period). In some embodiments, and during some treatment periods, LVOT gradient and LVEF outcomes are used in parallel to determine the dose adjustment, i.e., the dose for the next treatment period. For example, as shown in FIG. 2, if LVEF is below a safety threshold, then administration is discontinued during the next treatment period, regardless of LVOT gradient outcome; and if LVOT gradient is below a threshold, then a reduced dose (or alternatively, an increased dose) is administered during the next treatment period, without regard for LVEF outcome, unless LVEF outcome dictates that administration be discontinued. In some embodiments, LVOT gradient and LVEF outcomes are used in combination to determine the dose for the next treatment period. For example, referring again to FIG. 2, if LVEF is above a threshold and LVOT gradient is above a threshold, then the dose is increased; but if either LVEF is below a threshold or LVOT gradient is below a threshold, then the dose is maintained. In some embodiments, when LVEF is below a safety threshold (e.g., 50%), then administration is discontinued (temporarily or permanently) regardless of the LVOT gradient measured at the same assessment.

In some embodiments, the method does not comprise assessments of the pharmacokinetics of the myosin inhibitor in the patient during treatment, e.g., the method does not comprise assessments of the blood plasma concentration of the myosin inhibitor (e.g., mavacamten) during treatment.

In some embodiments, a pretreatment assessment (or “baseline” assessment) is performed. In some embodiments, the pre-treatment assessment is performed up to about 12 weeks prior to beginning administration of the myosin inhibitor (e.g., up to about 8 weeks, up to about 4 weeks, about 8-12 weeks, about 6-10 weeks, about 4-8 weeks, about 2-6 weeks, or about 1-4 weeks prior to beginning administration). The pretreatment assessment may include assessment of LVEF, e.g., by echocardiography. A pretreatment assessment outcome may be obtained from the pretreatment assessment. Some methods may further comprise determining whether the pretreatment assessment outcome is above or below a threshold. Some methods may further comprise determining whether the LVEF of the patient is below a pretreatment LVEF threshold. In some embodiments, the pretreatment LVEF threshold is a percentage value between 40% and 65%, e.g., about 60%, about 55%, about 52%, or about 50%. In some embodiments, the pretreatment LVEF threshold is about 55%. In some embodiments, the pretreatment LVEF threshold is about 50%. In some embodiments, when the LVEF from the pretreatment assessment is below the pretreatment LVEF threshold, then the myosin inhibitor is not administered to the patient, and when the LVEF from the pretreatment assessment is above the pretreatment LVEF threshold, then the myosin inhibitor is administered at the starting dose for a first treatment period.

In some embodiments, a first assessment is performed or obtained. Referring to FIG. 1, as a non-limiting example, the first assessment may be performed at or near the conclusion of a first treatment period. In some embodiments, the first assessment is performed during the final week of a first treatment period (e.g., as described herein.) For example, the first treatment period may be 28 days in duration and the first assessment outcome may be performed between days 22 and 28 of the first treatment period. In some embodiments, an assessment is taken during the final week of a treatment period. In some embodiments, the first assessment includes assessing left ventricular outflow tract obstruction by a non-invasive technique to obtain a first assessment outcome. The method may further include determining whether the first assessment outcome is above or below a threshold. As shown in FIG. 1, the first assessment may include assessing LVOT gradient. In some embodiments, the first assessment includes assessing LVOT gradient with Valsalva maneuver to obtain a first Valsalva LVOT gradient. In some embodiments, the non-invasive technique is echocardiography. In some embodiments, the non-invasive technique is an imaging technique. The method may further include determining whether the first Valsalva LVOT gradient is less than a Valsalva LVOT gradient threshold. The Valsalva LVOT gradient threshold may be a value between 20 mmHg and 30 mmHg, e.g., 20 mmHg, 25 mmHg, or 30 mmHg. In some embodiments, the Valsalva LVOT gradient threshold is about 20 mmHg.

In some embodiments, the first assessment includes (e.g., further includes) assessing LVEF to obtain a first LVEF. The method may also include determining whether the first LVEF above or below a LVEF threshold. The LVEF threshold may be a percentage value between 40% and 65%, e.g., about 60%, about 55%, about 52%, or about 50%. In some embodiments, the LVEF threshold is about 55%. In some embodiments, the LVEF threshold is about 50%. The first LVEF may be assessed using echocardiography. The first LVEF may be assessed using an imaging technique.

In some embodiments, a second assessment is performed or obtained. Referring to FIG. 1, the second assessment may be performed at or near the conclusion of a second treatment period. In some embodiments, the second assessment is performed during the final week of a second treatment period (e.g., as described herein.) In some embodiments, the second assessment includes assessing left ventricular outflow tract obstruction by a non-invasive technique to obtain a second assessment outcome. The method may further include determining whether the second assessment outcome above or below a threshold. As shown in FIG. 1, the second assessment may include assessing LVOT gradient. In some embodiments, the second assessment includes assessing LVOT gradient with Valsalva maneuver to obtain a second Valsalva LVOT gradient. In some embodiments, the non-invasive technique is echocardiography. In some embodiments, the non-invasive technique is an imaging technique. The method may further include determining whether the second Valsalva LVOT gradient is less than a Valsalva LVOT gradient threshold. The Valsalva LVOT gradient threshold may be a value between 20 mmHg and 30 mmHg, e.g., 20 mmHg, 25 mmHg, or 30 mmHg. In some embodiments, the Valsalva LVOT gradient threshold is about 20 mmHg.

In some embodiments, the second assessment includes (e.g., further includes) assessing LVEF to obtain a second LVEF. The method may also include determining whether the second LVEF above or below a LVEF threshold. The LVEF threshold may be a percentage value between 40% and 65%, e.g., about 60%, about 55%, about 52%, or about 50%. In some embodiments, the LVEF threshold is about 55%. In some embodiments, the LVEF threshold is about 50%. The second LVEF may be assessed using echocardiography. The second LVEF may be assessed using an imaging technique.

Additional assessments similar to the first and second assessments may also be performed or obtained, e.g., during an initiation phase.

In some embodiments, a third assessment is performed or obtained. In some embodiments, the third assessment is performed at or near the conclusion of a third treatment period (e.g., as described herein.) For example, the third assessment may be performed during the final week of the third treatment period. In some embodiments, the third assessment may be considered the transition point from the initiation phase to the maintenance phase. In some embodiments, the third assessment includes assessing left ventricular outflow tract obstruction by a non-invasive technique to obtain a third assessment outcome. The method may further include determining whether the third assessment outcome is above or below a threshold. In some embodiments, the third assessment includes assessing LVOT gradient with Valsalva maneuver to obtain a third Valsalva LVOT gradient. In some embodiments, the non-invasive technique is echocardiography. In some embodiments, the non-invasive technique is an imaging technique. The method may further include determining whether the third Valsalva LVOT gradient is greater than a Valsalva LVOT gradient threshold. The Valsalva LVOT gradient threshold may be a value between 20 mmHg and 35 mmHg, e.g., 20 mmHg, 25 mmHg, 30 mmHg or 35 mmHg.

In some embodiments, the third assessment includes assessing LVEF to obtain a third LVEF. The method may also include determining whether the third LVEF is above or below a LVEF threshold. The LVEF threshold may be a percentage value between 40% and 65%, e.g., about 60%, about 55%, about 52%, or about 50%. In some embodiments, the LVEF threshold is about 55%. In some embodiments, the LVEF threshold is about 50%. The third LVEF may be assessed using echocardiography. The third LVEF may be assessed using an imaging technique.

Additional assessments similar to the third assessment may also be performed or obtained, e.g., at or near the conclusion of a fourth or fifth treatment period (e.g., as described herein.)

The various assessments described herein may further include assessing other symptoms of the patient, e.g., other oHCM symptoms (such as new or worsening dyspnea, chest pain, fatigue, palpitations, leg edema or elevations in N terminal (NT) pro hormone b type natriuretic peptide (NT proBNP)).

Adjustments to the dosing of the myosin inhibitor may be made based on the one or more assessment outcomes, e.g., LVOT gradient(s) and/or LVEF(s), as described herein. In some embodiments, when the first assessment outcome is below a threshold LVOT gradient, then the starting dose is reduced, e.g., to a second dose. Referring to FIG. 1, the dose is decreased when LVOT gradient is less than a threshold value, and the dose is maintained when the LVOT gradient is greater than or equal to the threshold value. Referring to FIG. 4 as an example, when the first Valsalva LVOT gradient is less than 20 mmHg, then the starting dose of 5 mg QD mavacamten is reduced to 2.5 mg QD. The second dose (e.g., 2.5 mg QD of mavacamten) is then administered until another assessment is performed. Referring again to FIG. 1, in some embodiments, when the first assessment outcome is greater than or equal to a threshold LVOT gradient, then the starting dose is maintained, e.g., the second dose is the same as the starting dose. Referring again to FIG. 4 as an example, when the first Valsalva LVOT gradient is greater than or equal to 20 mmHg, then administration of 5 mg QD of mavacamten is continued until another assessment is performed.

In some embodiments, when the second assessment outcome is below a threshold LVOT gradient, then the second dose is reduced, e.g., to a third dose. Referring to FIG. 1, the dose is decreased, or interrupted, when LVOT gradient is less than a threshold value, and the dose is maintained when the LVOT gradient is greater than or equal to the threshold value. Referring to FIG. 4 as an example, at Week 8, the dose is decreased when Valsalva LVOT gradient is less than 20 mmHg, and the dose is maintained when the Valsalva LVOT gradient is greater than or equal to 20 mmHg. In some embodiments, when the second Valsalva LVOT gradient is less than 20 mmHg, then the dose of 2.5 mg QD mavacamten is reduced to 1 mg QD or reduced to 0 mg (or alternatively the dose of 5 mg QD mavacamten is reduced to 2.5 mg QD). The third dose (e.g., 0 or 1 mg QD of mavacamten, or alternatively 2.5 mg QD) is then administered until another assessment is performed. In some embodiments, when the second assessment outcome greater than or equal to a threshold LVOT gradient, then the second dose is maintained, e.g., the third dose is the same as the second dose. In some embodiments, when the second Valsalva LVOT gradient is greater than or equal to 20 mmHg, then then administration of the second dose (e.g., 2.5 or 5 mg QD of mavacamten) is continued (as the third dose) until another assessment is performed.

In some embodiments, when the third assessment outcome is above a threshold LVOT gradient, then the third dose is increased, e.g., to a fourth dose. FIG. 1 shows the dosing scheme proceeding to a maintenance phase. FIG. 2 shows the maintenance phase criteria whereby LVOT gradient guides the dose adjustment, for example, in combination with LVEF assessment. Dose may be increased when LVOT gradient is greater than or equal to a threshold value, if LVEF is greater than an LVEF threshold. Referring to FIG. 5 as an example, when the third Valsalva LVOT gradient is greater than or equal to 30 mmHg, then the third dose (e.g., 0 mg or 1 mg QD of mavacamten) is increased to 2.5 mg (or alternatively the dose of 2.5 mg is increased to 5 mg QD, or the dose of 5 mg is increased to 10 mg QD or 7.5 mg QD). The fourth dose (e.g., 2.5 mg QD of mavacamten, or alternatively 5, 7.5 or 10 mg QD) is then administered until another assessment is performed. In some embodiments, when the third assessment outcome is below a threshold LVOT gradient, then the third dose is maintained, e.g., the fourth dose is the same as the third dose. In some embodiments, when the third Valsalva LVOT gradient is less than 30 mmHg, then then administration of the third dose (e.g., 0, 1, 2.5 or 5 mg QD of mavacamten) is continued (as the fourth dose) until another assessment is performed.

In some embodiments, the third assessment includes assessing LVEF. In some embodiments, the LVEF is compared against a LVEF threshold (e.g., about 50%, about 52%, about 55%, or about 60%). In some embodiments, when the LVEF for the third assessment is less than the LVEF threshold (e.g., 55%), the dose is not increased, even if the Valsalva LVOT gradient is greater than or equal to a threshold Valsalva LVOT gradient (e.g., the Valsalva LVOT gradient is greater than or equal to 30 mmHg). FIG. 2 shows that both LVEF and LVOT gradient must be greater than equal to the respective thresholds for dose to be increased. Referring to FIG. 5 as an example, at Week 12, the dose is increased when Valsalva LVOT gradient is greater than or equal to 30 mmHg and LVEF is greater than or equal to 55%; otherwise, the dose is maintained (assuming criteria for temporary discontinuation (dose interruption) are not met).

In some embodiments, when the fourth assessment outcome is greater than or equal to a threshold LVOT gradient, then the fourth dose is increased, e.g., to a fifth dose. The criteria for this assessment and dose adjustment may also follow the maintenance phase criteria as shown in FIG. 2. In some embodiments, when the fourth Valsalva LVOT gradient is greater than or equal to 30 mmHg, then the dose of myosin inhibitor is increased (e.g., from 0 or 1 to 2.5 mg QD mavacamten, from 2.5 to 5 mg QD mavacamten, from 5 to 10 mg QD mavacamten, or from 10 to 15 mg QD mavacamten, or alternatively from 5 to 7.5 mg QD or from 7.5 to 10 mg QD). The fifth dose is then administered until another assessment is performed. In some embodiments, when the fourth assessment is below a threshold LVOT gradient, then the fourth dose is maintained, e.g., the fifth dose is the same as the fourth dose. In some embodiments, when the fourth Valsalva LVOT gradient is less than 30 mmHg, then then administration of the fourth dose (e.g., 0, 1, 2.5, 5, 7.5 or 10 mg QD of mavacamten) is continued (as the fifth dose) until another assessment is performed.

In some embodiments, the fourth assessment includes assessing LVEF. In some embodiments, the LVEF is compared against a LVEF threshold, e.g., as shown in FIG. 2 (e.g., about 50%, about 52%, about 55%, or about 60%). In some embodiments, when the LVEF for the fourth assessment in less than the LVEF threshold (e.g., 55%), the dose is not increased, even if the Valsalva LVOT gradient is greater than or equal to a threshold LVOT gradient (e.g., the Valsalva LVOT gradient is greater than or equal to 30 mmHg).

In some embodiments, when the dose of a myosin inhibitor is increased, e.g., during the maintenance phase, then an additional assessment is performed. In some embodiments, the additional assessment is an LVEF assessment. For example, when the dose of the myosin inhibitor is increased following the third assessment at the conclusion of the third treatment period (e.g., at the beginning of the maintenance phase), then an additional assessment of LVEF is performed during the fourth treatment period. In some embodiments, the additional assessment of LVEF is performed about 4 weeks after the dose increase.

Referring to FIG. 3, in some embodiments, when the assessment outcome includes LVEF and LVEF is less than a safety threshold (e.g., less than 50%), then administration of the myosin inhibitor is interrupted (temporarily discontinued). In some embodiments, when the assessment outcome includes LVEF and LVEF is great than or equal to a safety threshold (e.g., less than 50%), then administration of the myosin inhibitor is continued (e.g., at the same or a different dose). The LVEF assessment outcome may be determined at or near the conclusion of the first, second, third, fourth, or fifth treatment period. In some embodiments, the assessment for LVEF occurs at or near the conclusion of a treatment period (e.g., a first treatment period) and the temporary discontinuation comprises not administering the myosin inhibitor during the next treatment period (e.g., a second treatment period).

Still referring to FIG. 3, in some embodiments, following temporary discontinuation, administration of the myosin inhibitor is resumed after a subsequent LVEF assessment shows that LVEF is greater than or equal to a safety threshold (e.g., 50%). In some embodiments, administration is resumed at a lower dose than the dose received immediately prior to temporary discontinuation. In some embodiments, administration is resumed at the same dose as the dose received immediately prior to temporary discontinuation, e.g., when it is determined that discontinuation was due to transient factors (e.g., atrial fibrillation or other uncontrolled tachyarrhythmia or serious infection). In some embodiments, the dose is resumed at the minimum dose that is the lowest dose of the myosin inhibitor approved to be administered to patients by a governmental regulatory agency. In some embodiments, the governmental regulatory agency is an agency of the United States, European Union, Switzerland, Japan, China, South Korea, Canada, Mexico, Australia, New Zealand, Brazil, Russia, Ukraine, Georgia, Vietnam, Singapore, Malaysia, Philippines, India, Indonesia, Hong Kong, Israel, South Africa, Colombia, Costa Rica, Dominican Republic, Ecuador, Guatemala, El Salvador, Honduras, Egypt, Syria, Algeria, Kenya, Morocco, or Nigeria.

In some embodiments, when the assessment outcome includes LVEF and LVEF is less than a safety threshold (e.g., less than 50%), and administration of the myosin inhibitor was previously temporarily discontinued and then resumed, then administration of the myosin inhibitor is permanently discontinued.

Referring again to FIG. 3, in some embodiments, when the assessment outcome includes LVEF and LVEF is less than a safety threshold (e.g., less than 50%), and administration of the myosin inhibitor was previously temporarily discontinued due to an LVEF less than 50% during a previous assessment, and then previously resumed, then administration of the myosin inhibitor is permanently discontinued.

Referring to FIG. 6 as an example, in some embodiments, when the assessment outcome includes LVEF and LVEF is less than 50% and the patient was receiving 2.5 mg QD of mavacamten, and administration of the myosin inhibitor was previously temporarily discontinued due to an LVEF less than 50% during a previous assessment when the patient was receiving 2.5 mg QD of mavacamten, and then previously resumed at a dose of 2.5 mg QD of mavacamten, then administration of the myosin inhibitor is permanently discontinued.

Assessments may also be made in methods involving concomitant administration of a weak CYP2C19 inhibitor or moderate CYP3A4 inhibitor. In some embodiments, the assessment comprises assessing LVEF of the patient during the treatment period during which concomitant administration is initiated and results in temporarily discontinuing administration of the myosin inhibitor if LVEF is below a safety threshold. In some embodiments, the safety threshold is 50%. In some embodiments, the assessment comprises assessing LVEF and LVOT gradient of the patient after discontinuing administration and resuming administration when the LVOT gradient is above a threshold LVOT gradient and the LVEF is greater than or equal to a threshold LVEF. In some embodiments, the threshold LVOT gradient is 30 mmHg and the threshold LVEF is 55%. In some embodiments, assessing LVEF of the patient comprises assessing LVEF of the patient about four weeks after beginning the concomitant therapy, or during the fourth week following beginning the concomitant therapy.

In some embodiments, the present methods comprise setting, following, and/or enforcing protocols and limits with respect to the dispensing of the myosin inhibitor, or pharmaceutical composition thereof, to a pharmacy and to the patient. The protocols and limits may be part of a risk evaluation and mitigation strategy (REMS) program. In some embodiments, the purpose of the REMS program is to mitigate the risk of heart failure due to systolic dysfunction. In some embodiments, the REMS will educate prescribers, patients, and pharmacies on the risk of heart failure due to systolic dysfunction, certify prescribers and pharmacies in the REMS, enroll patients in the REMS, and restrict dispensing of mavacamten by only certified pharmacies to enrolled patients with prescriptions written by certified prescribers and for patients with a current patients status form (PSF), indicating that echocardiograms have been performed at the required frequency (e.g., as described herein for echocardiogram assessments of Valsalva LVOT gradient and LVEF). In some embodiments, mavacamten is distributed to certified pharmacies following REMS requirements.

In some embodiments, the present risk mitigation methods comprise submitting a patient status form (PSF) following an assessment as described herein. For example, following an echocardiogram for assessing LVOT gradient and/or LVEF, a PSF is completed and submitted to a risk management administrator. In some embodiments, the PSF is completed and submitted within 1, 2, or 3 days of an echocardiography assessment (e.g., within 2 days). In some embodiments, the PSF contains a statement by a doctor regarding the assessment outcomes, e.g., whether an LVOT gradient was above or below a threshold and/or whether an LVEF was above or below a threshold.

In some embodiments, the present risk mitigation methods comprise a dispensing limit, wherein a certain number of days of medication are requested, supplied, and/or received. In some embodiments, the dispensing limit for the myosin inhibitor (or pharmaceutical composition thereof) is a 28 to 90 day supply, or a 28 to 56 day supply, or a 30-40 day supply, e.g., a 35-day supply. For example, in some embodiments, the method comprises requesting a dispensing of the myosin inhibitor (or pharmaceutical composition thereof) to the pharmacy, hospital, doctor, or patient, wherein the amount requested is equal to the dispensing limit (e.g., a 35-day supply).

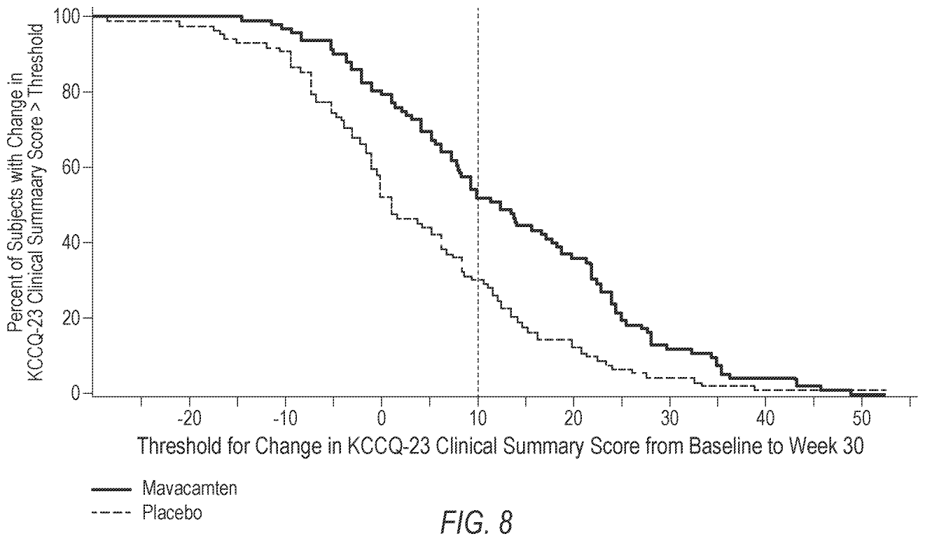

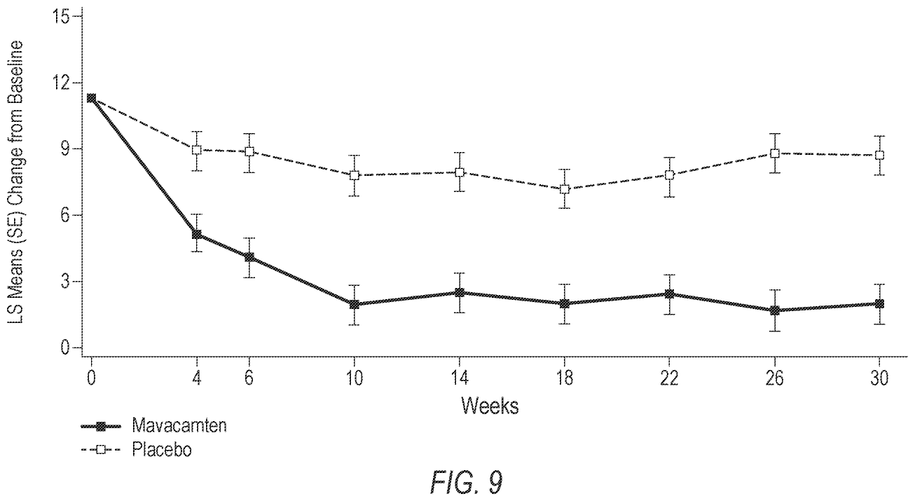

FIG. 8 shows a schedule for echocardiogram assessments, PSF submissions, and dispensing during the first 14 weeks following initiation of myosin inhibitor treatment. Implementation rules are set as follows. Each “Week X echo” should occur within that week (eg, the Week 4 echo should occur between Day 22 and Day 28). Prescribers will be educated to schedule their patients' Week 4, Week 8, and Week 12 echoes at treatment initiation to support scheduling approximately 4 weeks apart. Prescribers will be educated to submit their PSF within 2 days after the echo is performed. There will be a deadline programmed in the REMS administrator portal for PSF submission within 3 days following the echo deadline (eg, for Week 4, the deadline is Day 31). Dispense will be held until the PSF is received. Pharmacy authorization and dispense occurs once the PSF is received (eg, for Week 4, this can occur any time from Day 22 and Day 31). Pharmacies must confirm PSF submission and authorize dispense by the PSF submission deadline (eg, for Week 4, pharmacy must authorize dispense by Day 31). Based on this rule, patients will receive the new dispense from the day that the echo is performed to 4 days after the PSF submission deadline (based on the standard range of time from pharmacy authorization to patient receipt of drug-0 to 4 days with a 2-day average). A 35-day dispensing limit ensures no interruption in access to drug while the prescriber interprets the first echo, the PSF is submitted, and the dispense is authorized by the pharmacy and shipped to patient. FIG. 9 shows the schedule for echocardiogram assessments and dispensing during the first year following initiation of myosin inhibitor treatment. In Year 2 and beyond, dispensing will increase to a 90-day limit while the patient is on a stable dose (ie, no dose change within 12 weeks); if a patient requires a dose change, there will be a 35-day limit for that dispense. Once the patient is back on a stable dose, dispensing will return to a 90-day limit.

In some embodiments, disclosed herein is a method of controlling the distribution of a myosin inhibitor, the method comprising: certifying a healthcare provider and a pharmacy in a risk mitigation program; enrolling a patient in a risk mitigation program; receiving a patient status form with information regarding one or more echocardiogram assessments of the patient during treatment with the myosin inhibitor; receiving confirmation that screening for drug-drug interactions was performed; processing the patient status form; and authorizing dispensing of the myosin inhibitor to a pharmacy, wherein said authorization is subject to a dispense limit.

In some embodiments, the information regarding one or more echocardiogram assessments in the patient status form comprises (i) information regarding a Valsalva LVOT gradient of the patient during treatment with the myosin inhibitor and (ii) information regarding a LVEF of the patient during treatment with the myosin inhibitor. In some embodiments, the information regarding a Valsalva LVOT gradient is information regarding whether a Valsalva LVOT gradient has been determined. In some embodiments, the information regarding a Valsalva LVOT gradient is information regarding whether the Valsalva LVOT gradient is above or below one or more threshold(s). In some embodiments, the information regarding LVEF is information regarding whether an LVEF has been determined. In some embodiments, the information regarding LVEF is information regarding whether the LVEF is above or below one or more threshold(s). In some embodiments, the method further comprises providing information regarding drug-drug interactions. In some embodiments, the dispense limit is a 28 to 90 day supply, or a 28 to 56 day supply, or a 30-40 day supply, e.g., a 35-day supply of the myosin inhibitor to the pharmacy. In some embodiments, the patient status form must be submitted within a prescribed time window, e.g., at or near the conclusion of a treatment period as described herein. In some embodiments, the method includes verifying that the echocardiogram was performed during the prescribed time window, and/or that the patient status form with echocardiogram information was submitted during the prescribed time window. In some embodiments, if the patient status form is not received within the prescribed time window and/or if the echocardiogram was not performed within the prescribed time window, then dispensing is not authorized.

In some embodiments, the method further comprises withholding dispensing of drug until the patient status form is received. In some embodiments, the method further comprises withholding dispensing of drug until a complete patient status form is received and continuing to withhold dispensing of drug when an incomplete patient status form is received. In some embodiments, the method further comprises withholding dispensing of drug when the patient status form indicates that the LVEF of the patient is less than 50%. In some embodiments, the information regarding one or more echocardiogram assessments in the patient status form is received via a web-based portal. In some embodiments, receiving confirmation that screening for drug-drug interactions was performed comprises receiving confirmation from the pharmacy and/or the healthcare provider. In some embodiments, receiving confirmation that screening for drug-drug interactions was performed comprises receiving confirmation from both the pharmacy and the healthcare provider. In some embodiments, receiving confirmation that screening for drug-drug interactions was performed further comprises receiving a confirmation from the healthcare provider that no drug-drug interactions are present. In some embodiments, receiving confirmation that screening for drug-drug interactions was performed further comprises receiving a confirmation from the pharmacy that no drug-drug interactions are present.

In some embodiments, the patient status form is received via a data storage facility. The data storage facility may include a database of patient records, each patient record having a dispense authorization field for entering a first prescription of mavacamten. A system for the REMS may further include a central controller having one or more processors coupled to a communication network, which central controller is coupled to the data storage facility to read and write data to the data storage facility via a network. A REMS system may further include a drug storage facility having mavacamten stored therein. The central controller of the system may be programmed to monitor drug inventory in the drug storage facility and further programmed to control dispensing of mavacamten from the drug storage facility.

The central controller may control transmission and receipt of data to and from the data storage facility via the network. The central controller may be programed to output via the network a first dispense authorization of a first prescription of mavacamten to a specific patient previously subjected to an assessment (e.g., as described herein.) In some embodiments, output of the dispense authorization is dependent upon the results of the assessment—e.g., an echocardiogram, LVOT gradient, LVEF. The central controller may be further programmed to output a time period over which use of mavacamten by a patient is authorized and to schedule a subsequent assessment for the patient which subsequent test results for the patient are required to be received before authorizing additional mavacamten to be dispensed for the patient. The central controller may be programmed to take an action dependent upon subsequent test results selected from authorizing an additional prescription, a change to the prescription and output of a proposal for cessation of the prescription.

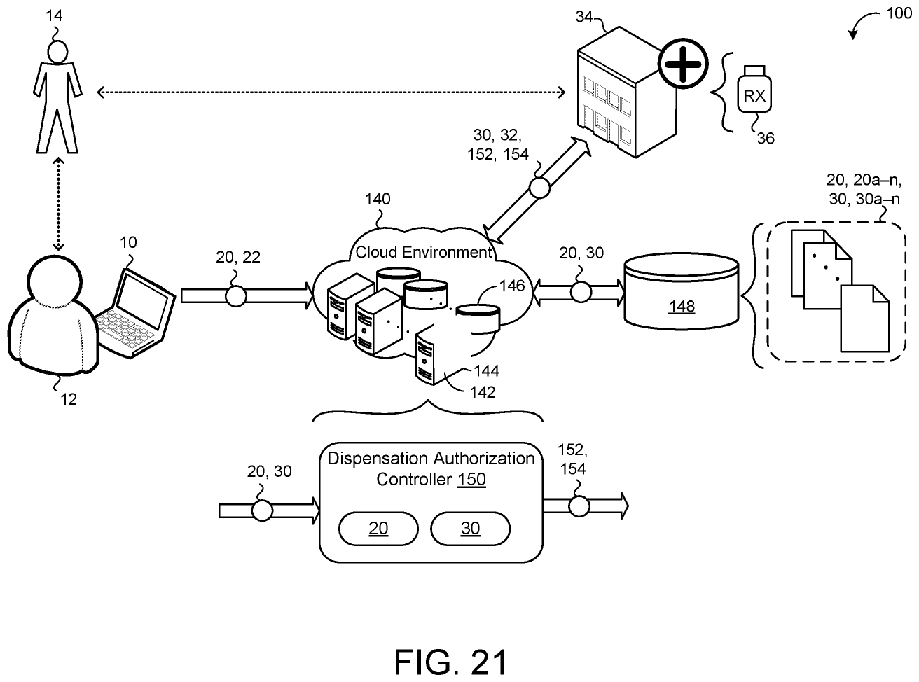

Referring to FIG. 21, in some implementations, an example medication dispensation authorization system 100 includes a remote system 140 in communication with one or more user devices 10 via, for example, one or more networks. The remote system 140 may be a single computer, multiple computers, or a distributed system (e.g., a cloud environment) having scalable/elastic resources 142 including computing resources 144 (e.g., data processing hardware) and/or storage resources 146 (e.g., memory hardware). A data store 148 (i.e., a remote storage device) may be overlain on the storage resources 146 to allow scalable use of the storage resources 146 by one or more of the clients (e.g., the user device 10) or the computing resources 144. Additionally or alternatively, the data store 148 is independent from the remote system 140 and the remote system 140 communications with the data store via, for example, a network.

The remote system 140 executes a dispensation authorization controller 150. The dispensation authorization controller 150 obtains or receives a healthcare professional (HCP) assessment record 20 associated with a patient 14. The HCP assessment record 20 includes a procedure result 22 or assessment as described herein. In some implementations, the procedure result 22 includes results of an echocardiogram. For example, the echocardiogram result includes a left ventricular ejection fraction (LVEF), a left ventricular outflow tract (LVOT) gradient, etc., derived from an echocardiogram procedure. In some examples, the HCP assessment record 20 includes risk events (e.g., one or more clinical heart failure events), and/or risks of potential drug-drug interactions. The dispensation authorization controller 150 may receive HCP assessment record 20 from a healthcare provider 12 via a user device 10 (e.g., at a doctor's office, hospital, or other healthcare provider facility).

The dispensation authorization controller 150 also obtains a pharmacy assessment record 30 associated with the patient 14. The pharmacy assessment record 30 includes a medical condition 32 of the patient 14 (e.g., heart conditions, allergies, etc.). Additionally or alternatively, the pharmacy assessment record 30 includes concomitant medications and supplements and/or potential drug-drug interactions. The dispensation authorization controller 150 may obtain or receive the pharmacy assessment record 30 from a pharmacy 34 or other entity on behalf of the pharmacy 34. The HCP assessment record 20 and/or the pharmacy assessment record 30 may be stored at the data store 148 along with any other number of other records 20, 30 for any number of patients 14. Healthcare providers 12, pharmacies 34, and any other entities may automatically upload records 20, 30 to the data store 148 as they become available. Alternatively, the dispensation authorization controller 150 may request the records 20, 30 (e.g., periodically or on demand) and store the received records 20, 30 at the data store 148.

The dispensation authorization controller 150 may determine, using the HCP assessment record 20, whether the patient 14 is authorized to receive a prescription authorization 152 authorizing the patient 14 use of a prescription medication 36. In some examples, the prescription medication includes a myosin inhibitor such as mavacamten. In some implementations, the dispensation authorization controller 150 determines whether the patient 14 is authorized to receive the prescription medication 36 in response to a prescription request (e.g., from the patient 14, the healthcare provider 12, and/or the pharmacy 34). In some examples, a healthcare provider (e.g., at the pharmacy 34, such as a pharmacist) determines whether a prescription authorization 152 is available (e.g., by querying the dispensation authorization controller 150 and/or the data store 148. When the healthcare provider determines that the patient 14 has a prescription authorization 152, the healthcare provider may provide the pharmacy assessment record 30 to the dispensation authorization controller 150. That is, in some examples, a healthcare provider (e.g., a pharmacist) provides the pharmacy assessment record 30 to the dispensation authorization controller 150 and/or the data store 148 in response to receiving or validating the prescription authorization 152.

When the patient 14 is authorized to receive the prescription authorization 152 (e.g., the procedure result 22 indicates that the patient 14 is a satisfactory candidate for the prescription medication 36), the dispensation authorization controller 150 determines, using the pharmacy assessment record 30, whether the pharmacy 34 is authorized to dispense the prescription medication 36 to the patient 14. For example, the dispensation authorization controller 150 determines whether the pharmacy assessment record 30 includes satisfactory pharmacy information (e.g., regarding the patient's medical conditions 32), concomitant medications and supplements, and/or potential drug-drug interactions (e.g., between different medications the patient 14 receives).

When the pharmacy 34 is authorized to dispense the prescription medication to the patient 14, the dispensation authorization controller 150 generates a dispensation authorization 154 and transmits the prescription authorization 152 and/or the dispensation authorization 154 to the pharmacy 34.

The pharmacy 34 may dispense or distribute the prescription medication 36 to the patient 14 as authorized by the dispensation authorization 154. For example, the dispensation authorization 154 includes a quantity of the prescription medication 36 the patient 14 is authorized to receive and/or a period of time (e.g., a dose schedule dictating times and/or frequencies to take the prescription medication 36) the patient 14 is authorized to receive the prescription medication 36. The dispensation authorization controller 150 may determine, using the HCP assessment record 20 and/or the pharmacy assessment record 30, that the dispensation authorization 154 indicates a non-standard supply of the prescription medication 36 (e.g., a quantity and/or frequency that is more or less than a standard supply or dose).

The pharmacy 34 may require an updated or new dispensation authorization 154 prior to adjusting the quantity, period of time, or any other parameters of the distribution of the prescription medication 36 to the patient 14. In some implementations, the dispensation authorization controller 150 (e.g., after the period of time has elapsed) determines whether an updated procedure result 22 associated with the patient 14 is available.

For example, the dispensation authorization controller 150 determines whether a new or updated procedure result 22 is available from the healthcare provider 12 or data store 148 (e.g., the patient 14 underwent a second procedure to obtain the updated procedure result 22). When the updated procedure result 22 is not available, the dispensation authorization controller 150 may decline to update the dispensation authorization 154, thus prohibiting the pharmacy from adjusting the quantity or period of time for the prescription medication 36 (e.g., deny any refills). When the updated procedure result 22 is available, the dispensation authorization controller 150 may update, using the updated procedure result, the dispensation authorization 154. For example, the updated dispensation authorization adjusts the quantity of the prescription medication 36 the patient 14 is authorized to receive and/or adjusts the period of time (e.g., the dose schedule) the patient 14 is authorized to receive the prescription medication 36.

In some implementations, when the patient 14 is not authorized to receive the prescription medication 36 and/or the pharmacy 34 is not authorized to dispense the prescription medication 36 to the patient 14, the dispensation authorization controller 150 generates a report. For example, when the HCP assessment record 20 and/or the pharmacy assessment record 30 includes unsatisfactory reporting information (e.g., regarding the experience of a clinical heart failure), the dispensation authorization controller 150 automatically generates a report including or referencing the unsatisfactory information and transmits the report to one or more regulatory agencies.

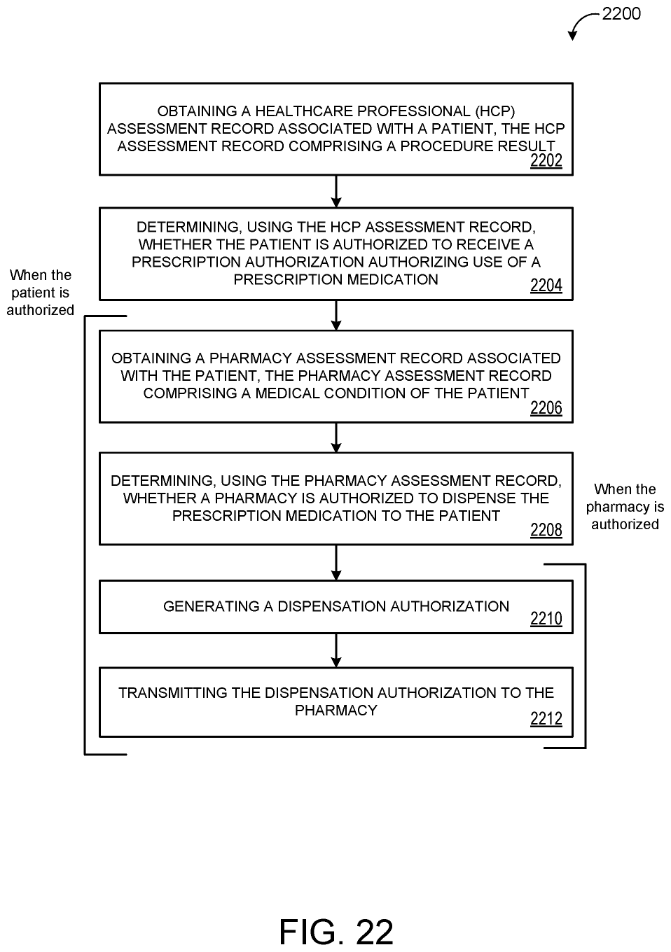

FIG. 22 is a flowchart of an exemplary arrangement of operations for a computer-implemented method 2200 that when executed by data processing hardware 144 causes the data processing hardware 144 to perform operations. The method 2200, at operation 2202 includes obtaining an HCP assessment record 20 associated with a patient 14. The HCP assessment record includes a procedure result 22. At operation 2204, the method 2200 includes determining, using the HCP assessment record 20, whether the patient 14 is authorized to receive a prescription authorization 152 authorizing use of a prescription medication 36. When the patient 14 is authorized to receive the prescription medication 36, the method 2200 includes, at operation 2206, obtaining a pharmacy assessment record 30 associated with the patient 14. The pharmacy assessment record 30 includes a medical condition 32 of the patient 14. At operation 2208, the method 2200 includes determining, using the pharmacy assessment record 30, whether a pharmacy 34 is authorized to dispense the prescription medication 36 to the patient 14. When the pharmacy 34 is authorized to dispense the prescription medication 36 to the patient 14, the method 2200 also includes, at operation 2210, generating a dispensation authorization 154 and, at operation 2212, transmitting the dispensation authorization 154 to the pharmacy 34.

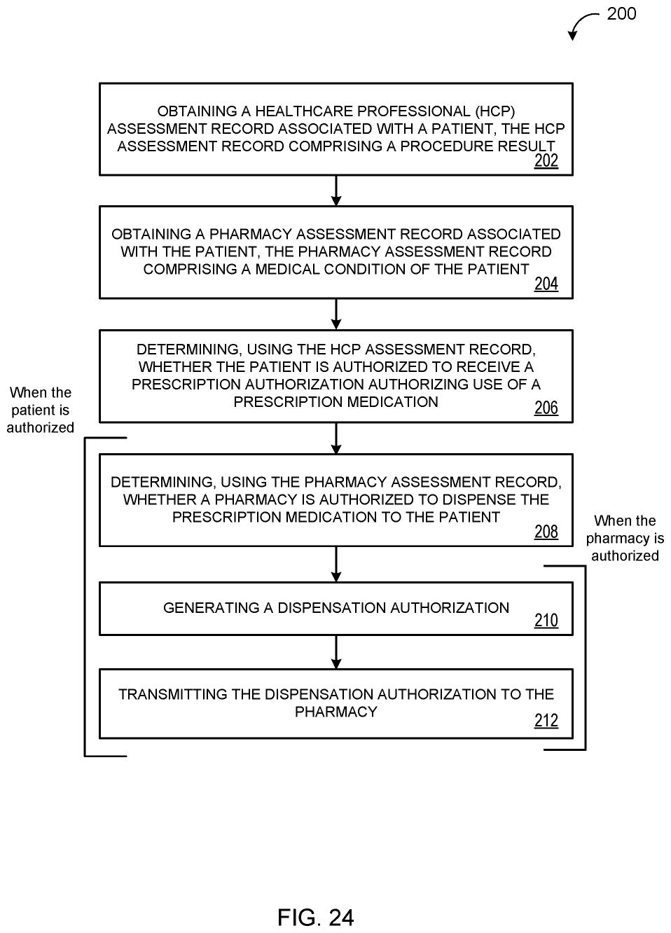

FIG. 24 is a flowchart of an alternative exemplary arrangement of operations for a computer-implemented method 200 that when executed by data processing hardware 144 causes the data processing hardware 144 to perform operations. The method 200, at operation 202 includes obtaining an HCP assessment record 20 associated with a patient 14. The HCP assessment record includes a procedure result 22. At operation 204, the method 200 includes obtaining a pharmacy assessment record 30 associated with the patient 14. The pharmacy assessment record 30 includes a medical condition 32 of the patient 14. At operation 206, the method 200 includes determining, using the HCP assessment record 20, whether the patient 14 is authorized to receive a prescription authorization 152 authorizing use of a prescription medication 36. When the patient 14 is authorized to receive the prescription medication 36, the method 200 includes, at operation 208, determining, using the pharmacy assessment record 30, whether a pharmacy 34 is authorized to dispense the prescription medication 36 to the patient 14 and, when the pharmacy 34 is authorized to dispense the prescription medication 36 to the patient 14, at operation 210, generating a dispensation authorization 154 and, at operation 212, transmitting the dispensation authorization 154 to the pharmacy 34.

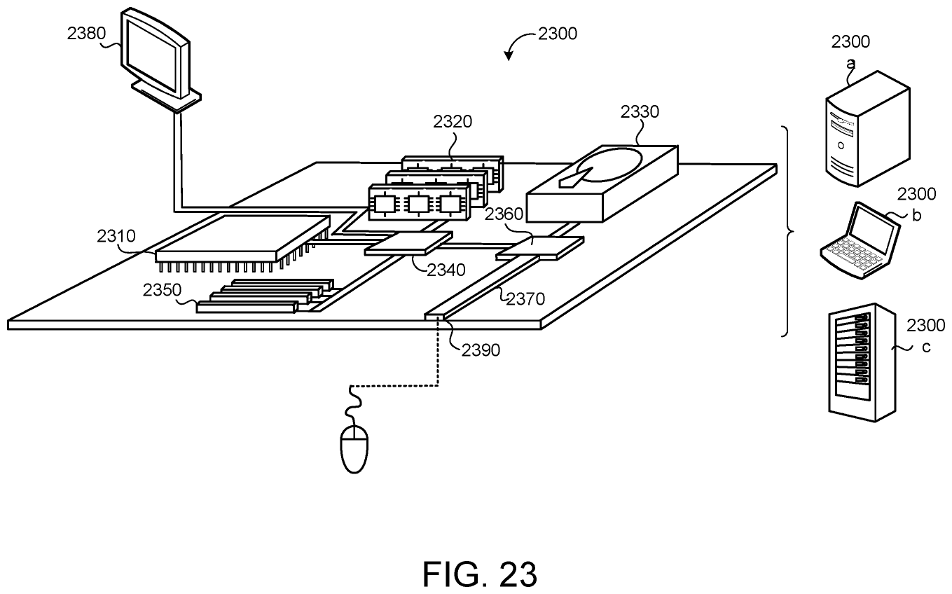

FIG. 23 is a schematic view of an example computing device 2300 that may be used to implement the systems and methods described in this document. The computing device 2300 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 2300 includes a processor 2310, memory 2320, a storage device 2330, a high-speed interface/controller 2340 connecting to the memory 2320 and high-speed expansion ports 2350, and a low speed interface/controller 2360 connecting to a low speed bus 2370 and a storage device 2330. Each of the components 2310, 2320, 2330, 2340, 2350, and 2360, are interconnected using various busses, and may be mounted on a common motherboard or in other manners as appropriate. The processor 2310 can process instructions for execution within the computing device 2300, including instructions stored in the memory 2320 or on the storage device 2330 to display graphical information for a graphical user interface (GUI) on an external input/output device, such as display 2380 coupled to high speed interface 2340. In other implementations, multiple processors and/or multiple buses may be used, as appropriate, along with multiple memories and types of memory. Also, multiple computing devices 2300 may be connected, with each device providing portions of the necessary operations (e.g., as a server bank, a group of blade servers, or a multi-processor system).

The memory 2320 stores information non-transitorily within the computing device 2300. The memory 2320 may be a computer-readable medium, a volatile memory unit(s), or non-volatile memory unit(s). The non-transitory memory 2320 may be physical devices used to store programs (e.g., sequences of instructions) or data (e.g., program state information) on a temporary or permanent basis for use by the computing device 2300. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 2330 is capable of providing mass storage for the computing device 2300. In some implementations, the storage device 2330 is a computer-readable medium. In various different implementations, the storage device 2330 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 2320, the storage device 2330, or memory on processor 2310.

The high speed controller 2340 manages bandwidth-intensive operations for the computing device 2300, while the low speed controller 2360 manages lower bandwidth-intensive operations. Such allocation of duties is exemplary only. In some implementations, the high-speed controller 2340 is coupled to the memory 2320, the display 2380 (e.g., through a graphics processor or accelerator), and to the high-speed expansion ports 2350, which may accept various expansion cards (not shown). In some implementations, the low-speed controller 2360 is coupled to the storage device 2330 and a low-speed expansion port 2390. The low-speed expansion port 2390, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 2300 may be implemented in a number of different forms, as shown in the figure. For example, it may be implemented as a standard server 2300a or multiple times in a group of such servers 2300a, as a laptop computer 2300b, or as part of a rack server system 2300c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Further aspects and embodiments of the invention are described below.

In one aspect, described herein is a method of treating a patient in need thereof with a myosin inhibitor, comprising:
administering a starting dose of the myosin inhibitor to the patient for a first treatment period;
when a first measurement of left ventricular outflow tract obstruction of the patient taken at or near the conclusion of the first treatment period is below a threshold value, administering a first reduced dose of the myosin inhibitor to the patient during a second treatment period wherein the first reduced dose is less than the starting dose; and
when a second measurement of left ventricular outflow tract obstruction of the patient taken at or near the conclusion of the second treatment period is below a threshold value, administering a second reduced dose of the myosin inhibitor to the patient during a third treatment period, wherein the second reduced dose is less than a dose of the myosin inhibitor administered immediately prior to the second reduced dose.

In some embodiments, the method further comprises:
obtaining a first measurement of left ventricular outflow tract obstruction of the patient taken at or near the conclusion of the first treatment period; and
obtaining a second measurement of left ventricular outflow tract obstruction of the patient taken at or near the conclusion of the second treatment period.

In some embodiments, the second treatment period immediately follows the first treatment period.

In some embodiments, the third treatment period immediately follows the second treatment period.

In some embodiments, the dose of the myosin inhibitor administered to the patient is not increased until after the third treatment period.

In some embodiments, the first and second measurements are taken using echocardiography.

In some embodiments, the first measurement of left ventricular outflow tract obstruction is a measurement of Valsalva LVOT gradient.

In some embodiments, the second measurement of left ventricular outflow tract obstruction is a measurement of Valsalva LVOT gradient.

In some embodiments, the threshold value is a Valsalva LVOT gradient of 20 mmHg.

In some embodiments, the patient's risk of an adverse event is reduced as compared to continued administration of the myosin inhibitor at the starting dose.

In some embodiments, the first treatment period is about 4 weeks.

In some embodiments, the second treatment period is about 4 weeks.

In some embodiments, the first, second, and third treatment periods are each about 4 weeks.

In some embodiments, the patient is suffering from symptomatic obstructive hypertrophic cardiomyopathy.

In some embodiments, the patient is suffering from symptomatic New York Heart Association (NYHA) class II-III obstructive hypertrophic cardiomyopathy.

In some embodiments, the myosin inhibitor is selected from the group consisting of a compound of group (I), a compound of group (II), a compound of group (III), mavacamten, MYK-581, aficamten, and pharmaceutically acceptable salts thereof.

In some embodiments, the myosin inhibitor is mavacamten or a pharmaceutically acceptable salt thereof.

In some embodiments, the myosin inhibitor is mavacamten.

In some embodiments, the starting dose is about 5 mg per day of mavacamten.

In some embodiments, the first reduced dose is less than about 5 mg per day of mavacamten.

In some embodiments, the first reduced dose is selected from the group consisting of about 2.5 mg per day of mavacamten, about 1 mg per day of mavacamten, or 0 mg per day of mavacamten.

In some embodiments, the second reduced dose is about 1 mg per day of mavacamten or 0 mg per day of mavacamten.

In some embodiments, the first reduced dose is about 2.5 mg per day of mavacamten and the second reduced dose is 0 mg per day of mavacamten.

In some embodiments, a left ventricular ejection fraction of the patient at or near the conclusion of the first and second treatment periods is greater than or equal to about 50%.

Another aspect disclosed herein is a method of treating symptomatic obstructive hypertrophic cardiomyopathy in a patient in need thereof, comprising:
administering a starting dose of 5 mg per day of mavacamten to the patient for a first treatment period, wherein the first treatment period is about 4 weeks;
administering 2.5 mg per day of mavacamten to the patient for a second treatment period when a Valsalva left ventricular outflow tract (LVOT) gradient of the patient taken at or near the conclusion of the first treatment period is below 20 mmHg, wherein the second treatment period is about 4 weeks; and
administrating 0 mg per day of mavacamten to the patient for a third treatment period when a Valsalva left ventricular outflow tract (LVOT) gradient of the patient taken at or near the conclusion of the second treatment period is below 20 mmHg, wherein the third treatment period is about 4 weeks.

In some embodiments, the second treatment period immediately follows the first treatment period.

In some embodiments, the third treatment period immediately follows the second treatment period.

In some embodiments, the method further comprises administering 2.5 mg per day of mavacamten to the patient for a fourth treatment period when a measurement of left ventricular ejection fraction of the patient taken at or near the conclusion of the third treatment period is greater than or equal to about 50%, wherein the fourth treatment period is about 4 weeks

In some embodiments, the fourth treatment period immediately follows the third treatment period.

In some embodiments, the patient has a LVEF of greater than or equal to about 50%.

Another aspect described herein is a method of treating a patient in need thereof with a myosin inhibitor, the method comprising the steps of:
(a) administering a starting dose of the myosin inhibitor during a first treatment period;
(b) assessing the patient for left ventricular outflow tract obstruction to obtain a first assessment outcome and determining whether the first assessment outcome is below a first threshold value;
(c) when the first assessment outcome is below a first threshold value, administering a second dose during a second treatment period, wherein the second dose is less than the starting dose;
(d) assessing the patient for left ventricular outflow tract obstruction to obtain a second assessment outcome and determining whether the second assessment outcome is below a second threshold value; and
(e) when the second assessment outcome is below a second threshold value, administering a third dose during a third treatment period, wherein the third dose is less than the second dose.

In some embodiments, the method further comprises the steps of:
(f) at or near the conclusion of the third treatment period, assessing the patient for left ventricular outflow tract obstruction to obtain a third assessment outcome and determining whether the third assessment outcome is greater than or equal to a third threshold value, and assessing the left ventricular ejection fraction (LVEF) of the patient; and
(g) when the third assessment outcome is greater than or equal to a third threshold value and the LVEF of the patient is greater than or equal to a LVEF threshold, administering a fourth dose during a fourth treatment period, wherein the fourth dose is greater than the third dose.

In some embodiments, the method further comprises the steps of:
(f) at or near the conclusion of the third treatment period, assessing the left ventricular ejection fraction (LVEF) of the patient; and
(g) when the LVEF of the patient is greater than or equal to a safety threshold, administering a fourth dose during a fourth treatment period, wherein the fourth dose is greater than the third dose.

In some embodiments, the method further comprises the steps of:
(h) at or near the conclusion of the fourth treatment period, assessing the patient for left ventricular outflow tract obstruction to obtain a fourth assessment outcome and determining whether the fourth assessment outcome is greater than or equal to a fourth threshold value, and assessing the left ventricular ejection fraction (LVEF) of the patient; and
(i) when the fourth assessment outcome is greater than or equal to the fourth threshold value and the LVEF of the patient is greater than or equal to a LVEF threshold, administering a fifth dose during a fifth treatment period, wherein the fifth dose is greater than the fourth dose.

In some embodiments, the first and second assessments are performed by a non-invasive technique.

In some embodiments, the third assessment is performed by a non-invasive technique.

In some embodiments, the non-invasive technique comprises echocardiography

In some embodiments, the non-invasive technique comprises a cardiac imaging technique.

In some embodiments, the non-invasive technique comprises measurement of LVOT gradient with Valsalva maneuver.

In some embodiments, the first assessment outcome is a first Valsalva LVOT gradient and the second assessment outcome is a second Valsalva LVOT gradient.

In some embodiments, the first threshold value and the second threshold value are each a Valsalva LVOT gradient.

In some embodiments, the first threshold value and the second threshold value are each a Valsalva LVOT gradient of 20 mmHg.

In some embodiments, the method mitigates the risk of an adverse event.

In some embodiments, the adverse event is systolic dysfunction.

In some embodiments, the adverse event is heart failure.

In some embodiments, the patient's risk of the adverse event is reduced as compared to continued administration of the myosin inhibitor at the starting dose.

In some embodiments, the myosin inhibitor is selected from the group consisting of a compound of group (I), a compound of group (II), a compound of group (III), mavacamten, MYK-581, and aficamten, and pharmaceutically acceptable salts thereof.

In some embodiments, the myosin inhibitor is mavacamten or a pharmaceutically acceptable salt thereof.

In some embodiments, the myosin inhibitor is mavacamten.

In some embodiments, the starting dose is 5 mg per day of mavacamten.

In some embodiments, the second dose is less than 5 mg per day of mavacamten.

In some embodiments, the second dose is 2.5 mg per day of mavacamten.

In some embodiments, the third dose is less than 2.5 mg per day of mavacamten.

In some embodiments, the third dose is 0 mg per day of mavacamten or 1 mg per day of mavacamten.

In some embodiments, the patient is suffering from obstructive hypertrophic cardiomyopathy (oHCM).

In some embodiments, the patient is suffering from symptomatic New York Heart Association (NYHA) class II-III oHCM.

In some embodiments, a dose of 0 mg is administered during the third treatment period.

In some embodiments, the dose of the myosin inhibitor administered to the patient is not increased until after the third treatment period.

In some embodiments, the LVEF threshold is 55%.

In some embodiments, the safety threshold is 50%.

In some embodiments, the method further comprises assessing the patient for left ventricular ejection fraction (LVEF) at or near the conclusion of the first treatment period and at or near the conclusion of the second treatment period.

In some embodiments, the method further comprises temporarily discontinuing treatment when the LVEF assessment at or near the conclusion of the first or second treatment period is less than 50%.

In some embodiments, the first treatment period is about four weeks and the second treatment period is about four weeks.

In some embodiments, the first treatment period is about four weeks, the second treatment period is about four weeks, and the third treatment period is about four weeks.

Yet another aspect disclosed herein is a method treating a patient in need thereof with mavacamten, the method comprising the steps of:
(a) administering 5 mg per day of mavacamten to the patient during a first treatment period;
(b) assessing the patient for LVOT gradient with Valsalva maneuver to determine a first Valsalva LVOT gradient;
(c) administering 2.5 mg per day of mavacamten per day to the patient during a second treatment period when the first Valsalva LVOT gradient is below 20 mmHg;
(d) assessing the patient for LVOT gradient with Valsalva maneuver to determine a second Valsalva LVOT gradient; and
(e) administering 0 mg or 1 mg of mavacamten per day to the patient for a third treatment period when the second Valsalva LVOT gradient is below 20 mmHg.

In some embodiments, the method further comprises the steps of:
(f) at or near the conclusion of the third treatment period, assessing the patient for LVOT gradient with Valsalva maneuver to determine a third Valsalva LVOT gradient and assessing the left ventricular ejection fraction (LVEF) of the patient; and
(g) administering 2.5 mg of mavacamten per day to the patient for a fourth treatment period when the third Valsalva LVOT gradient is greater than or equal to 30 mmHg and the LVEF of the patient is greater than or equal to 55%.

In some embodiments, the method further comprises the steps of:
(f) at or near the conclusion of the third treatment period, assessing the left ventricular ejection fraction (LVEF) of the patient; and
(g) administering 2.5 mg of mavacamten per day to the patient for a fourth treatment period when the LVEF of the patient is greater than or equal to 50%.

In some embodiments, the method further comprises the steps of:
(h) at or near the conclusion of the fourth treatment period, assessing the patient for LVOT gradient with Valsalva maneuver to determine a fourth Valsalva LVOT gradient and assessing the left ventricular ejection fraction (LVEF) of the patient; and
(i) administering 5 mg of mavacamten per day to the patient for a fifth treatment period when the fourth Valsalva LVOT gradient is greater than or equal to 30 mmHg and the LVEF of the patient is greater than or equal to 55%.

Another aspect disclosed herein is a method of administering mavacamten to a patient, wherein the patient is suffering from oHCM, comprising the steps of:
(a) administering to the patient a starting dose of 5 mg per day of mavacamten for a first treatment period;
(b) assessing the patient for LVOT gradient with Valsalva maneuver to determine a first Valsalva LVOT gradient;
(c) administering to the patient 2.5 mg per day of mavacamten for a second treatment period when the first Valsalva LVOT gradient is less than 20 mmHg;
(d) assessing the patient for LVOT gradient with Valsalva maneuver to determine a second Valsalva LVOT gradient;
(e) administering to the patient 0 mg per day of mavacamten for a third treatment period when the second Valsalva LVOT gradient is less than 20 mmHg;
(f) assessing the patient to determine a first left ventricular ejection fraction (LVEF); and
(g) administering to the patient 2.5 mg per day of mavacamten for a fourth treatment period when the first LVEF is greater than or equal to 50%.

In some embodiments, the method further comprises the steps of:
(h) assessing the patient for LVOT gradient with Valsalva maneuver to determine a third Valsalva LVOT gradient and assessing the patient to determine a second left ventricular ejection fraction (LVEF); and
(i) administering to the patient 5 mg per day of mavacamten for a fifth treatment period when the third Valsalva LVOT gradient is greater than or equal to 30 mmHg and the second LVEF is greater than or equal to 55%.

In some embodiments, a risk of systolic dysfunction and/or heart failure in the patient is reduced as compared to continued administration of the myosin inhibitor at the starting dose.

In some embodiments, the first treatment period is about four weeks and the second treatment period is about four weeks.

In some embodiments, the third treatment period is about four weeks.

In some embodiments, the fourth treatment period is about twelve weeks.

In some embodiments, the patient is suffering from symptomatic New York Heart Association (NYHA) class II-III oHCM.

Still another aspect disclosed herein is a method of administering mavacamten to a patient, wherein the patient is suffering from oHCM, comprising the steps of:
administering to the patient a starting dose of 5 mg per day of mavacamten for a first treatment period;
assessing the patient for LVOT gradient with Valsalva maneuver to determine a first Valsalva LVOT gradient;
administering a second dose of mavacamten during a second treatment period, wherein if the first Valsalva LVOT gradient is less than 20 mmHg, then the second dose is 2.5 mg per day, and wherein if the first Valsalva LVOT gradient is greater than or equal to 20 mmHg, then the second dose is 5 mg per day;
assessing the patient for LVOT gradient with Valsalva maneuver to determine a second Valsalva LVOT gradient; and
administering a third dose of mavacamten during a third treatment period, wherein if the second Valsalva LVOT gradient is less than 20 mmHg, then the third dose is less than the second dose and the third dose is 2.5 mg, 1 mg, or 0 mg per day; and wherein if the first Valsalva LVOT gradient is greater than or equal to 20 mmHg, then the third dose is the same as the second dose and the third dose is 5 mg or 2.5 mg per day.

In some embodiments, the patient receives a third dose of 0 mg per day during the third treatment period; the method further comprising the steps of:
assessing the patient to determine a first left ventricular ejection fraction (LVEF); and
administering a fourth dose of mavacamten during a fourth treatment period, wherein if the first LVEF is greater than or equal to 50%, then the fourth dose is 2.5 mg per day, and wherein if the first LVEF is less than 50%, then the fourth dose is 0 mg per day.

In some embodiments, the patient receives a third dose of 1 mg per day, 2.5 mg per day, or 5 mg per day during the third treatment period; the method further comprising the steps of:
assessing the patient for LVOT gradient with Valsalva maneuver to determine a third Valsalva LVOT gradient and assessing the patient to determine a first left ventricular ejection fraction (LVEF); and
administering a fourth dose of mavacamten during a fourth treatment period, wherein if the third Valsalva LVOT gradient is greater than or equal to 30 mmHg and the first LVEF is greater than or equal to 55%, then the fourth dose is greater than the third dose and the fourth dose is 2.5 mg, 5 mg, or 10 mg per day, and wherein if the third Valsalva LVOT gradient is less than 30 mmHg or the first LVEF is less than 55%, then the fourth dose is the same as the third dose and the fourth dose is 1 mg, 2.5 mg, or 5 mg per day.

In some embodiments, a risk of systolic dysfunction and/or heart failure in the patient is reduced as compared to if the patient received continued administration of mavacamten at the starting dose.

In some embodiments, the first treatment period is about four weeks and the second treatment period is about four weeks.

In some embodiments, the third treatment period is about four weeks.

In some embodiments, the fourth treatment period is about twelve weeks.

In some embodiments, the patient is suffering from symptomatic New York Heart Association (NYHA) class II-III oHCM.

Still another aspect disclosed herein is a method of treating a patient in need thereof with a myosin inhibitor, comprising:
administering a starting dose of a myosin inhibitor to the patient for a first treatment period;
administering a second dose of the myosin inhibitor to the patient for a second treatment period, wherein:
if a LVOT gradient of the patient taken at or near the conclusion of the first treatment period is below a threshold value, then the second dose is less than the starting dose, and
if a LVOT gradient of the patient taken at or near the conclusion of the first treatment period is greater than or equal to the threshold value, then the second dose is the same as the starting dose; and
administering a third dose of the myosin inhibitor to the patient for a third treatment period, wherein:
if a LVOT gradient of the patient taken at or near the conclusion of the second treatment period is below the threshold value, then the third dose is less than the second dose, and
if a LVOT gradient of the patient taken at or near the conclusion of the second treatment period is greater than or equal to the threshold value, then the third dose is the same as the second dose.

In some embodiments, the second treatment period immediately follows the third treatment period.

In some embodiments, the third treatment period immediately follows the second treatment period.

In some embodiments, the method further comprises administering a fourth dose of the myosin inhibitor to the patient for a fourth treatment period, wherein:
if the third dose is less than the second dose, and the second dose is less than the starting dose, and a measurement of left ventricular ejection fraction of the patient taken at or near the conclusion of the third treatment period is greater than or equal to about 50%, then the fourth dose is the same as the lowest previously administered dose and the fourth treatment period, and
if the third dose is equal to the second dose and/or the second dose is equal to the starting dose, then the fourth treatment period is longer than the third treatment period.

In some embodiments, the fourth treatment period immediately follows the third treatment period.

In some embodiments, the first, second, and third treatment periods are about four weeks.

In some embodiments, the threshold value is 20 mmHg.

In some embodiments, the fourth treatment period is about 4 weeks when the third dose is less than the second dose, and the second dose is less than the starting dose, and a measurement of LVEF of the patient taken at or near the conclusion of the third treatment period is greater than or equal to about 50%.

In some embodiments, the fourth treatment period is about 12 weeks when the third dose is equal to the second dose and/or the second dose is equal to the starting dose.

In some embodiments, the patient has a LVEF of greater than or equal to about 50%.

In some embodiments, the LVOT gradient is a Valsalva LVOT gradient.

In some embodiments, the patient's risk of an adverse event is reduced as compared to continued administration of the myosin inhibitor at the starting dose.

In some embodiments, the first, second, and third treatment periods are each about 4 weeks.

In some embodiments, the patient is suffering from symptomatic obstructive hypertrophic cardiomyopathy.

In some embodiments, the patient is suffering from symptomatic New York Heart Association (NYHA) class II-III obstructive hypertrophic cardiomyopathy.

In some embodiments, the myosin inhibitor is selected from the group consisting of a compound of group (I), a compound of group (II), a compound of group (III), mavacamten, MYK-581, aficamten, and pharmaceutically acceptable salts thereof.

In some embodiments, the myosin inhibitor is mavacamten or a pharmaceutically acceptable salt thereof.

In some embodiments, the myosin inhibitor is mavacamten.

In some embodiments, the starting dose is about 5 mg per day of mavacamten.

In some embodiments, the second dose is less than about 5 mg per day of mavacamten.

In some embodiments, the third dose is less than about 2.5 mg per day of mavacamten.

In some embodiments, the second dose is selected from the group consisting of about 2.5 mg per day of mavacamten, about 1 mg per day of mavacamten, or 0 mg per day of mavacamten.

In some embodiments, the third dose is about 1 mg per day of mavacamten or 0 mg per day of mavacamten.

In some embodiments, the second dose is about 2.5 mg per day of mavacamten and the third dose is 0 mg per day of mavacamten.

In some embodiments, the fourth dose is selected from the group consisting of 2.5 mg, 5 mg, and 10 mg per day of mavacamten.

Also disclosed herein is a method of treating a patient in need thereof with a myosin inhibitor, the method comprising:
administering a starting dose of the myosin inhibitor at least once per day at the start of an initiation phase; and
performing one or more assessments of the patient for left ventricular outflow tract obstruction during the initiation phase to obtain one or more assessment outcomes; and
discontinuing administration of the myosin inhibitor based on the one or more assessment outcomes.

In some embodiments, the method further comprises resuming administration of the myosin inhibitor after the discontinuation.

In some embodiments, administration is resumed following an assessment of LVEF of the patient, wherein administration is resumed when LVEF is greater than or equal to a safety threshold.

In some embodiments, the one or more assessments are performed by a non-invasive technique.

In some embodiments, the non-invasive technique comprises echocardiography.

In some embodiments, the non-invasive technique comprises a cardiac imaging technique.

In some embodiments, the non-invasive technique comprises measurement of LVOT gradient with Valsalva maneuver and the one or more assessment outcomes are one or more Valsalva LVOT gradients.

In some embodiments, the method comprises discontinuing administration of the myosin inhibitor when a Valsalva LVOT gradient is below 20 mmHg.

In some embodiments, the method comprises performing two or more assessments of the patient for left ventricular outflow obstruction by a non-invasive technique during the initiation phase to obtain two or more assessment outcomes.

In some embodiments, the non-invasive technique comprises measurement of LVOT gradient with Valsalva maneuver and the two or more assessment outcomes are two or more Valsalva LVOT gradients.

In some embodiments, the method comprises discontinuing administration of the myosin inhibitor when at least two of the two or more Valsalva LVOT gradients are below 20 mmHg.

In some embodiments, the method mitigates the patient's risk of an adverse event.

In some embodiments, the adverse event is systolic dysfunction.

In some embodiments, the adverse event is heart failure.

In some embodiments, the patient's risk of the adverse event is reduced as compared to continued administration of the myosin inhibitor.

In some embodiments, the myosin inhibitor is selected from the group consisting of a compound of group (I), a compound of group (II), a compound of group (III), mavacamten, MYK-581, and aficamten, optionally as a pharmaceutically acceptable salt thereof.

In some embodiments, the myosin inhibitor is mavacamten or a pharmaceutically acceptable salt thereof.

In some embodiments, the myosin inhibitor is mavacamten.

In some embodiments, the starting dose is 5 mg per day of mavacamten.

In some embodiments, the patient is suffering from oHCM.

In some embodiments, the initiation phase is from about 4 weeks to about 6 months in duration.

In some embodiments, the initiation phase is from about 8 weeks to about 16 weeks in duration.

In some embodiments, the safety threshold is 50%.

Also disclosed herein is a method of treating a patient in need thereof with mavacamten, comprising the steps of:
(a) administering a starting dose of 5 mg per day of mavacamten to the patient at the start of an initiation phase; and
(b) performing two or more assessments of the patient for LVOT gradient with Valsalva maneuver at separate times during the initiation phase to obtain two or more Valsalva LVOT gradients; and
(c) discontinuing administration of mavacamten when each of the two or more Valsalva LVOT gradients is below 20 mmHg.

Another aspect disclosed herein is a method of treating a patient in need thereof with a myosin inhibitor, the method comprising:
administering a starting dose of the myosin inhibitor to the patient;
assessing the patient for left ventricular outflow tract obstruction at or near the conclusion of two or more separate treatment periods to obtain two or more assessment outcomes; and
administering a first reduced dose and subsequently administering a second reduced dose, based upon the two or more assessment outcomes, wherein said assessment outcomes are below a threshold value, wherein the first reduced dose is less than the starting dose, and the second reduced dose is less than the first reduced dose.

In some embodiments, the dose of the myosin inhibitor administered to the patient is not increased until the two or more assessment outcomes are completed at or near the conclusion of the two or more separate treatment periods.

In some embodiments, the two or more assessments are performed by a non-invasive technique.

In some embodiments, the non-invasive technique comprises echocardiography.

In some embodiments, the non-invasive technique comprises a cardiac imaging technique.

In some embodiments, the non-invasive technique comprises measurement of LVOT gradient with Valsalva maneuver.

In some embodiments, the assessment outcome is a Valsalva LVOT gradient.

In some embodiments, the threshold value is a Valsalva LVOT gradient.

In some embodiments, the threshold value is a Valsalva LVOT gradient of 20 mmHg.

In some embodiments, the method mitigates the risk of an adverse event.

In some embodiments, the adverse event is systolic dysfunction.

In some embodiments, the adverse event is heart failure.

In some embodiments, the patient's risk of the adverse event is reduced as compared to continued administration of the myosin inhibitor at the starting dose.

In some embodiments, the myosin inhibitor is selected from the group consisting of a compound of group (I), a compound of group (II), a compound of group (III), mavacamten, MYK-581, aficamten, and pharmaceutically acceptable salts thereof.

In some embodiments, the myosin inhibitor is mavacamten or a pharmaceutically acceptable salt thereof.

In some embodiments, the myosin inhibitor is mavacamten.

In some embodiments, the starting dose is 5 mg per day of mavacamten.

In some embodiments, the first reduced dose is less than 5 mg per day.

In some embodiments, the second reduced dose is less than 2.5 mg per day.

In some embodiments, the first reduced dose is 2.5 mg per day of mavacamten.

In some embodiments, the second reduced dose is 1 mg per day or 0 mg per day of mavacamten.

In some embodiments, the patient is suffering from obstructive hypertrophic cardiomyopathy (oHCM).

In some embodiments, the patient is suffering from symptomatic New York Heart Association (NYHA) class II-III oHCM.

In some embodiments, the two or more separate treatment periods comprise a first treatment period, and a second treatment period, and wherein the two or more assessment outcomes comprise a first assessment outcome at or near the conclusion of the first treatment period and a second assessment outcome at or near the conclusion of the second treatment period.

In some embodiments, the method further comprises administering the myosin inhibitor for a third treatment period, following the second assessment.

In some embodiments, the dose of the myosin inhibitor administered to the patient is not increased until after the third treatment period.

In some embodiments, the first treatment period is about four weeks and the second treatment period is about four weeks.

In some embodiments, the third treatment period is about four weeks.

In some embodiments, the method comprises assessing the left ventricular ejection fraction (LVEF) of the patient at or near the conclusion of the two or more separate treatment periods.

Yet another aspect disclosed herein is a method of mitigating a risk of heart failure with reduced ejection fraction due to administration of a myosin inhibitor to a patient, the method comprising the steps of:
administering a myosin inhibitor to the patient;
temporarily discontinuing administration of the myosin inhibitor when the patient has a LVEF of less than 50%;
resuming administration of the myosin inhibitor to the patient when the patient has a LVEF of greater than or equal to 50%; and
permanently discontinuing administration of the myosin inhibitor when the patient has a LVEF of less than 50% after resuming administration.

In some embodiments, the LVEF is determined by a non-invasive technique.

In some embodiments, the non-invasive technique is echocardiography.

In some embodiments, the non-invasive technique comprises a cardiac imaging technique.

In some embodiments, resuming administration comprises administering the same dose that the patient received prior to temporary discontinuation.

In some embodiments, resuming administration comprises administering a lower dose than the dose the patient received prior to temporary discontinuation.

In some embodiments, resuming administration comprises administering a minimum dose of myosin inhibitor to the patient, wherein the minimum dose is the lowest dose of the myosin inhibitor approved to be administered to patients by a governmental regulatory agency.

In some embodiments, the governmental regulatory agency is an agency of the United States, European Union, Switzerland, Japan, China, South Korea, Canada, Mexico, Australia, New Zealand, Brazil, Russia, Ukraine, Georgia, Vietnam, Singapore, Malaysia, Philippines, India, Indonesia, Hong Kong, Israel, South Africa, Colombia, Costa Rica, Dominican Republic, Ecuador, Guatemala, El Salvador, Honduras, Egypt, Syria, Algeria, Kenya, Morocco, or Nigeria.

In some embodiments, the myosin inhibitor is selected from the group consisting of a compound of group (I), a compound of group (II), a compound of group (III), mavacamten, MYK-581, and aficamten, and pharmaceutically acceptable salts thereof.

In some embodiments, the myosin inhibitor is mavacamten or a pharmaceutically acceptable salt thereof.

In some embodiments, the myosin inhibitor is mavacamten. 171. In some embodiments, the patient is suffering from obstructive hypertrophic cardiomyopathy (oHCM).

In some embodiments, the patient is suffering from symptomatic New York Heart Association (NYHA) class II-III oHCM.

Also disclosed herein is a method of mitigating a risk of heart failure with reduced ejection fraction due to administration of mavacamten to a patient, the method comprising the steps of:
administering mavacamten to the patient at a dose of 2.5 mg per day;
temporarily discontinuing administration of mavacamten when the patient has a LVEF of less than 50%;
resuming administration of mavacamten to the patient at a dose of 2.5 mg per day when the patient has a LVEF of greater than or equal to 50%; and
permanently discontinuing administration of mavacamten when the patient has a LVEF of less than 50% after resuming administration.

Also disclosed herein is a method of treating obstructive hypertrophic cardiomyopathy (oHCM) in a patient in need thereof, the method comprising administering a therapeutically effective amount of mavacamten to the patient, wherein the patient does not receive concomitant administration of a strong or moderate CYP2C19 inducer nor a strong or moderate CYP3A4 inducer.

Also disclosed herein is a method of treating obstructive hypertrophic cardiomyopathy (oHCM) in a patient in need thereof, where the patient is being treated with a strong or moderate CYP2C19 inducer or a strong or moderate CYP3A4 inducer, the method comprising:
discontinuing administration to the patient of the strong or moderate CYP2C19 inducer or strong or moderate CYP3A4 inducer; and
administering a therapeutically effective amount of mavacamten to the patient, thereby avoiding the use of mavacamten in combination with a strong or moderate CYP2C19 inducer or a strong or moderate CYP3A4 inducer.

Another aspect disclosed herein is a method of administering a myosin inhibitor to a patient who initiates concomitant therapy with a weak CYP2C19 inhibitor or a moderate CYP3A4 inhibitor while receiving myosin inhibitor therapy, the method comprising:
administering a first daily dose of the myosin inhibitor during a first treatment period prior to initiating concomitant therapy with a weak CYP2C19 inhibitor or a moderate CYP3A4 inhibitor;
administering a second daily dose of the myosin inhibitor, which is less than the first daily dose, during a second treatment period, wherein the patient receives concomitant therapy with a weak CYP2C19 inhibitor or a moderate CYP3A4 inhibitor during the second treatment period.

In some embodiments, the method further comprises assessing LVEF of the patient during the second treatment period and temporarily discontinuing administration of the myosin inhibitor if LVEF is below a safety threshold.

In some embodiments, the safety threshold is 50%.

In some embodiments, the method further comprises assessing LVEF and LVOT gradient of the patient after discontinuing administration, and resuming administration of the first daily dose when the LVOT gradient is greater than or equal to a threshold value and the LVEF is greater than or equal to a LVEF threshold.

In some embodiments, the threshold value is 30 mmHg and the LVEF threshold is 55%.

In some embodiments, the myosin inhibitor is selected from the group consisting of a compound of group (I), a compound of group (II), a compound of group (III), mavacamten, MYK-581, and aficamten, and pharmaceutically acceptable salts thereof.

In some embodiments, the myosin inhibitor is mavacamten or a pharmaceutically acceptable salt thereof.

In some embodiments, the myosin inhibitor is mavacamten.

In some embodiments, the first daily dose is 5 mg, 10 mg, or 15 mg of mavacamten, and the second daily dose is 2.5 mg, 5 mg, or 10 mg of mavacamten.

In some embodiments, the weak CYP2C19 inhibitor or moderate CYP3A4 inhibitor is selected from the group consisting of cimetidine, ciprofloxacin, diltiazem, felbamate, omeprazole at a dose of 20 mg once daily, isoniazid, fluconazole, and verapamil.

In some embodiments, the patient is suffering from obstructive hypertrophic cardiomyopathy (oHCM).

In some embodiments, the patient is suffering from symptomatic New York Heart Association (NYHA) class II-III oHCM.

In some embodiments, assessing LVEF of the patient during the second treatment period comprises assessing LVEF of the patient about four weeks after beginning the concomitant therapy.

In some embodiments, the second treatment period is at least 12 weeks, and wherein the second daily dose is not increased to a higher dose during at least the first 12 weeks of the second treatment period.

Yet another aspect disclosed herein is a method of treating HCM in a patient being administered a first daily dose of mavacamten, wherein said patient is then in need of being treated concurrently with a weak CYP2C19 inhibitor or a moderate CYP3A4 inhibitor in addition to the mavacamten, comprising:
administering to the patient a second daily dose of mavacamten, which is less than the first daily dose, in addition to administration of the weak CYP2C19 inhibitor or moderate CYP3A4 inhibitor.

In some embodiments, the first daily dose is 5 mg, 10 mg, or 15 mg per day and the second daily dose is 2.5 mg, 5 mg, or 10 mg per day.

Still another aspect disclosed herein is a method of initiating concomitant administeration of mavacamten to a patient being administered a weak CYP2C19 inhibitor or a moderate CYP3A4 inhibitor, wherein the patient is in need of concomitant administration of mavacamten and the weak CYP2C19 inhibitor or the moderate CYP3A4 inhibitor and wherein the patient is on a stable therapy of the weak CYP2C19 inhibitor or the moderate CYP3A4 inhibitor, the method comprising:
concomitantly administering a daily dose of 5 mg per day of mavacamten and the stable therapy of the weak CYP2C19 inhibitor or the moderate CYP3A4 inhibitor to the patient.

Still another aspect disclosed herein is a method of administering a myosin inhibitor to a patient who initiates or increases the dose of a concomitant therapy with a negative inotrope while receiving myosin inhibitor therapy, the method comprising:
(a) administering a therapeutically effective amount of a myosin inhibitor during a first treatment period;
(b) continuing to administer the myosin inhibitor, during a second treatment period, wherein the patient initiates or increases the dose of a concomitant therapy with a negative inotrope during the second treatment period; and
(c) providing echocardiographic monitoring of LVEF during the second treatment period.

In some embodiments, echocardiographic monitoring of LVEF is provided until stable doses and clinical response have been achieved.

In some embodiments, the method further comprises providing close medical supervision during the second treatment period.

In some embodiments, the myosin inhibitor is mavacamten.

Another aspect disclosed herein is a computer-implemented method when executed by data processing hardware causes the data processing hardware to perform operations comprising:
obtaining a healthcare professional (HCP) assessment record associated with a patient, the HCP assessment record comprising a procedure result;
determining, using the HCP assessment record, whether the patient is authorized to receive a prescription authorization authorizing use of a prescription medication; and
when the patient is authorized to receive the prescription medication:
obtaining a pharmacy assessment record associated with the patient, the pharmacy assessment record comprising a medical condition of the patient;
determining, using the pharmacy assessment record, whether a pharmacy is authorized to dispense the prescription medication to the patient; and
when the pharmacy is authorized to dispense the prescription medication to the patient:
generating a dispensation authorization; and
transmitting the dispensation authorization to the pharmacy.

In some embodiments, the procedure result comprises an echocardiogram result.

In some embodiments, at least one of the HCP assessment record and the pharmacy assessment record comprises potential drug-drug interactions.

In some embodiments, obtaining the HCP assessment record comprises retrieving the HCP assessment record from a database remote from the data processing hardware.

In some embodiments, determining whether the patient is authorized to receive the prescription authorization is in response to receiving a prescription request.

In some embodiments, the dispensation authorization comprises:
a quantity of the prescription medication the patient is authorized to receive; and
a period of time the patient is authorized to receive the prescription medication.

In some embodiments, the operations further comprise, after the period of time has elapsed:
determining whether an updated procedure result associated with the patient is available;
when the updated procedure result is available, updating, using the updated procedure result, the dispensation authorization; and
when the updated procedure result is unavailable, declining to update the dispensation authorization.

In some embodiments, updating the dispensation authorization comprises at least one of:
adjusting the quantity of the prescription medication the patient is authorized to receive; and
adjusting the period of time the patient is authorized to receive the prescription medication.

In some embodiments, the operations further comprise, when the patient is not authorized to receive the prescription medication or when the pharmacy is not authorized to dispense the prescription medication to the patient, generating a report for a regulatory agency.

In some embodiments, the prescription medication comprises mavacamten.

Also disclosed herein is a system comprising:
data processing hardware; and
memory hardware in communication with the data processing hardware, the memory hardware storing instructions that when executed on the data processing hardware cause the data processing hardware to the perform operations recited above.

Also disclosed herein is a method of mitigating a risk of heart failure due to systolic dysfunction in a patient being administered a myosin inhibitor, comprising:
providing a data storage facility comprising a database comprising patient HCP assessment records and patient pharmacy assessment records, wherein each patient HCP assessment record comprises information on the patient's date of an echocardiogram, LVEF determined from the echocardiogram, VLVOT determined from the echocardiogram, experience of a clinical heart failure event, and risk of potential drug-drug interactions, and wherein each patient pharmacy assessment record comprises information on the patient's medical conditions, concomitant medications and supplements, and potential drug-drug interactions;
providing a central controller having one or more processors coupled to a communication network, which central controller is coupled to the data storage facility to read and write data to the data storage facility via the network, wherein:
the central controller controls transmission and receipt of data to and from the data storage facility via the network,
the central controller being programed to output via the network a HCP authorization for prescription of the myosin inhibitor to the patient, wherein output of the HCP authorization is dependent upon satisfactory HCP information on the date of echocardiogram, the echocardiogram outcomes, the experience of a clinical heart failure event, and the risk of drug-drug interactions entered into each patient HCP assessment record, and wherein the central controller inhibits the HCP authorization output for unsatisfactory HCP information,
the central controller being further programed to output via the network a dispensation authorization for the myosin inhibitor to the patient, wherein output of the pharmacy authorization is dependent upon output of the HCP authorization and satisfactory pharmacy information on the patient's medical conditions, concomitant medications and supplements, and potential drug-drug interactions entered into each patient pharmacy assessment record, and wherein the central controller inhibits the dispensation authorization output for unsatisfactory pharmacy information and/or lack of HCP authorization, and
wherein the central controller manages one or more aspects reporting unsatisfactory information on the experience of a clinical heart failure event to a regulatory agency, or other overseeing body.

Also disclosed herein is a computer-implemented method when executed by data processing hardware causes the data processing hardware to perform operations comprising:
obtaining a healthcare professional (HCP) assessment record associated with a patient, the HCP assessment record comprising a procedure result;
obtaining a pharmacy assessment record associated with the patient, the pharmacy assessment record comprising a medical condition of the patient;
determining, using the HCP assessment record, whether the patient is authorized to receive a prescription authorization authorizing use of a prescription medication; and
when the patient is authorized to receive the prescription medication:
determining, using the pharmacy assessment record, whether a pharmacy is authorized to dispense the prescription medication to the patient; and
when the pharmacy is authorized to dispense the prescription medication to the patient:
   generating a dispensation authorization; and
   transmitting the dispensation authorization to the pharmacy.

In some embodiments, the procedure result comprises an echocardiogram result.

In some embodiments, at least one of the HCP assessment record and the pharmacy assessment record comprises potential drug-drug interactions.

In some embodiments, obtaining the HCP assessment record comprises retrieving the HCP assessment record from a database remote from the data processing hardware.

In some embodiments, determining whether the patient is authorized to receive the prescription authorization is in response to receiving a prescription request.

In some embodiments, the dispensation authorization comprises:
a quantity of the prescription medication the patient is authorized to receive; and
a period of time the patient is authorized to receive the prescription medication.

In some embodiments, the operations further comprise, after the period of time has elapsed:
determining whether an updated procedure result associated with the patient is available;
when the updated procedure result is available, updating, using the updated procedure result, the dispensation authorization; and
when the updated procedure result is unavailable, declining to update the dispensation authorization.

In some embodiments, updating the dispensation authorization comprises at least one of:
adjusting the quantity of the prescription medication the patient is authorized to receive; and
adjusting the period of time the patient is authorized to receive the prescription medication.

In some embodiments, the operations further comprise, when the patient is not authorized to receive the prescription medication or when the pharmacy is not authorized to dispense the prescription medication to the patient, generating a report for a regulatory agency.

In some embodiments, the prescription medication comprises mavacamten.

Also disclosed herein is a system comprising:
data processing hardware; and
memory hardware in communication with the data processing hardware, the memory hardware storing instructions that when executed on the data processing hardware cause the data processing hardware to the perform operations recited in any of the above methods.

### EXAMPLES

### Example 1. Dosing and Administration of Mavacamten

FIGS. 4-6 shows a dosing scheme for the present example. During an initiation phase (FIG. 4), a patient having obstructive HCM is given an initial dose of 5 mg of mavacamten for once daily (QD) oral administration for weeks 1-4. During the fourth week, the patient is assessed by echocardiography. Specifically, the Valsalva LVOT gradient and LVEF of the patient are determined. As shown in FIG. 5, if echocardiography at this visit, or any other visit, shows LVEF<50%, then treatment is interrupted (i.e., temporarily discontinued) for 4 weeks. After 4 weeks of treatment interruption, another echocardiogram is taken, and if LVEF is ≥50%, then treatment is resumed at one dose level below the previous dose level. Referring again to FIG. 4, at the 4 week visit, Valsalva LVOT gradient (VLVOT) is determined and if VLVOT is <20 mmHg, then the dose is reduced to 2.5 mg QD. If VLVOT is ≥20 mmHg at the 4 week visit, then the dose is maintained at 5 mg QD.

After an additional 4 weeks, at the 8 Week visit (during the 8^{th} week), the patient is assessed again by echocardiography. Again, treatment is interrupted (i.e., temporarily discontinued) for at least 4 weeks if LVEF is <50%. If VLVOT is <20 mmHg, then the dose is reduced—patients that were receiving 2.5 mg QD reduce their dose to 0 mg QD (i.e., withhold drug) and patients that were receiving 5 mg QD reduce their dose to 2.5 mg QD. If VLVOT is ≥20 mmHg, then the dose is maintained (i.e., at 2.5 mg QD or 5 mg QD). Thus, the dose is not increased at the Week 4 or Week 8 visits.

After an additional 4 weeks, at the 12 Week visit (during the 12^{th} week), the patient is assessed again by echocardiography. Again, treatment is interrupted if LVEF is less than 50%. For patients receiving 5 mg QD or 2.5 mg QD at the time of the 12 Week visit, the dose is increased one level if LVEF≥55% and VLVOT≥30 mmHg. Dose levels are 0 mg, 2.5 mg, 5 mg, 10 mg, and 15 mg (QD). Thus, for example, a patient receiving 5 mg QD before the Week 12 visit, who has LVEF≥55% and VLVOT≥30 mmHg, will be increased to 10 mg QD following the Week 12 visit. For patients on interruption/withholding (0 mg) at the time of the 12 Week visit, they will be restarted on 2.5 mg QD if LVEF≥50%.

If the dose is increased (including from 0 mg to 2.5 mg QD), then the patient will have another visit 4 weeks after the dose increase to assess LVEF. The patient will continue on the same dose for the next 8 weeks unless LVEF<50%. Beginning at Week 12, the patient will have a clinical visit every 12 weeks during which LVEF and VLVOT will be determined and dose can be increased one level if LVEF≥55% and VLVOT≥30 mmHg. The maximum dose of mavacamten is 15 mg QD. Treatment is permanently discontinued if LVEF<50% at 2.5 mg QD for two times during treatment.

Concomitant administration of mavacamten with moderate and strong inhibitors of CYP2C19 is contraindicated. Concomitant administration of mavacamten with strong inhibitors of CYP3A4 is contraindicated. Concomitant administration of mavacamten with moderate and strong inducers of CYP3A4 or CYP2C19 is contraindicated.

MAVACAMTEN capsules for oral use

WARNING: RISK OF HEART FAILURE
MAVACAMTEN can cause heart failure due to systolic dysfunction.
Echocardiogram assessments of left ventricular ejection fraction (LVEF) required before and during MAVACAMTEN use.
Initiation in patients with LVEF<55% not recommended. Interrupt if LVEF<50% or if worsening clinical status.
Certain CYP 450 inhibitors inducers are contraindicated in patients taking MAVACAMTEN because of an increased risk of heart failure.
MAVACAMTEN is available only through a restricted program called the MAVACAMTEN REMS Program.

Indications and Usage

MAVACAMTEN is a cardiac myosin inhibitor indicated for the treatment of adults with symptomatic New York Heart Association (NYHA) class II-III obstructive hypertrophic cardiomyopathy (HCM) to improve functional capacity and symptoms.

Dosage and Administration

Dosage must be individualized based on clinical status and echocardiographic assessment of patient response. Refer to the Full Prescribing Information for instructions.

Dosage Forms and Strengths

Capsules: 2.5 mg, 5 mg, 10 mg, and 15 mg

Contraindications
Moderate to strong CYP2C19 inhibitors or strong CYP3A4 inhibitors
Moderate to strong CYP2C19 inducers or moderate to strong CYP3A4 inducers

Warnings and Precautions
Heart Failure: Consider interruption of MAVACAMTEN in patients with intercurrent illness.
Drug Interactions Leading to Heart Failure or Loss of Effectiveness: Advise patients of the potential for drug interactions including with over-the-counter medications.
Embryo-Fetal Toxicity: May cause fetal harm. Advise females of reproductive potential to use effective contraception until 4 months after the last dose. Use a contraceptive not affected by CYP 450 enzyme induction or add nonhormonal contraception.

Adverse Reactions

Adverse reactions occurring in >5% of patients and more commonly on MAVACAMTEN than on placebo were dizziness (27%) and syncope (6%).

Drug Interactions
Weak CYP2C19 inhibitors and moderate CYP3A4 inhibitors: May increase risk of heart failure. If initiating an inhibitor, MAVACAMTEN dose reduction and additional monitoring are required.
Negative inotropes: Close medical supervision and LVEF monitoring is recommended if a negative inotrope is initiated, or the dose of a negative inotrope is increased. Avoid certain combinations of negative inotropes.

Full Prescribing Information

Warning: Risk of Heart Failure

MAVACAMTEN reduces left ventricular ejection fraction (LVEF) and can cause heart failure due to systolic dysfunction.

Echocardiogram assessments of LVEF are required prior to and during treatment with MAVACAMTEN. Initiation of MAVACAMTEN in patients with LVEF<55% is not recommended. Interrupt MAVACAMTEN if LVEF is <50% at any visit or if the patient experiences heart failure symptoms or worsening clinical status.

Concomitant use of MAVACAMTEN with certain cytochrome P450 inhibitors or discontinuation of certain cytochrome P450 inducers may increase the risk of heart failure due to systolic dysfunction; therefore, the use of MAVACAMTEN is contraindicated with the following:
Moderate to strong CYP2C19 inhibitors or strong CYP3A4 inhibitors
Moderate to strong CYP2C19 inducers or moderate to strong CYP3A4 inducers

Because of the risk of heart failure due to systolic dysfunction, MAVACAMTEN is available only through a restricted program under a Risk Evaluation and Mitigation Strategy (REMS) called MAVACAMTEN REMS PROGRAM.

1. Indications and Usage

MAVACAMTEN is indicated for the treatment of adults with symptomatic New York Heart Association (NYHA) class II-III obstructive hypertrophic cardiomyopathy (HCM) to improve functional capacity and symptoms.

2. Dosage and Administration

2.1. Initiation, Maintenance, and Interruption of Treatment

Confirm absence of pregnancy and usage of effective contraception in females of reproductive potential.

Initiation or up-titration of MAVACAMTEN in patients with LVEF<55% is not recommended.

The recommended starting dose is 5 mg once daily without regard to food; allowable subsequent doses with titration are 2.5, 5, 10, or 15 mg once daily.

Patients may develop heart failure while taking MAVACAMTEN. Regular LVEF and Valsalva left ventricular outflow tract (LVOT) gradient assessment is required for careful titration to achieve an appropriate target Valsalva LVOT gradient, while maintaining LVEF≥50% and avoiding heart failure symptoms (see FIG. 4 and FIG. 5).

Daily dosing takes weeks to reach steady-state drug levels and therapeutic effects, and genetic variation in metabolism and drug interactions can cause large differences in exposure.

When initiating or titrating MAVACAMTEN, first consider LVEF then consider the Valsalva LVOT gradient and patient clinical status to guide appropriate MAVACAMTEN dosing. Follow the algorithms for Initiation (FIG. 4) and Maintenance (FIG. 5) for appropriate MAVACAMTEN dosing and monitoring schedules.

If LVEF<50% while taking MAVACAMTEN, interrupt treatment. Follow the algorithm for Interruption (FIG. 6) for guidance on interrupting, restarting, or discontinuing MAVACAMTEN. If interrupted at 2.5 mg, either restart at 2.5 mg or discontinue permanently.

FIG. 4 shows an Initiation Phase. FIG. 5 shows a Maintenance Phase. FIG. 6 shows Treatment Interruption at Any Clinic Visit if LVEF<50%.

Delay dose increases when there is intercurrent illness (e.g., serious infection) or arrhythmia (e.g., atrial fibrillation or other uncontrolled tachyarrhythmia) that may impair systolic function. Consider interruption of MAVACAMTEN in patients with intercurrent illness.

Missed or Delayed Doses

If a dose is missed, it should be taken as soon as possible, and the next scheduled dose should be taken at the usual time the following day. Exact timing of dosing during the day is not essential, but two doses should not be taken on the same day.

Swallow capsules whole. Do not break, open, or chew the capsules.

2.2. Concomitant Administration of Weak CYP2C19 or Moderate CYP3A4 Inhibitors

Initiate MAVACAMTEN at the recommended starting dosage of 5 mg orally once daily in patients who are on stable therapy with a weak CYP2C19 inhibitor or a moderate CYP3A4 inhibitor.

Reduce dosage of MAVACAMTEN by one level (i.e., 15 to 10 mg, 10 to 5 mg, or 5 to 2.5 mg) in patients who initiate a weak CYP2C19 inhibitor or a moderate CYP3A4 inhibitor. Schedule clinical and echocardiographic assessment 4 weeks after inhibitor initiation, and do not up-titrate MAVACAMTEN until 12 weeks after inhibitor initiation. Avoid initiation of concomitant weak CYP2C19 and moderate CYP3A4 inhibitors in patients who are on stable treatment with 2.5 mg of MAVACAMTEN because a lower MAVACAMTEN once-daily dose is not available.

3. Dosage Forms and Strengths

MAVACAMTEN is available as capsules imprinted with the strength and “Maya” in the following strengths:
2.5 mg—light purple cap
5 mg—yellow cap
10 mg—pink cap
15 mg—gray cap

4. Contraindications

MAVACAMTEN is contraindicated with concomitant use of: Moderate to strong CYP2C19 inhibitors or strong CYP3A4 inhibitors Moderate to strong CYP2C19 inducers or moderate to strong CYP3A4 inducers

5. Warnings and Precautions

5.1. Heart Failure

MAVACAMTEN reduces systolic contraction and can cause heart failure or totally block ventricular function. Patients who experience a serious intercurrent illness (e.g., serious infection) or arrhythmia (e.g., atrial fibrillation or other uncontrolled tachyarrhythmia) are at greater risk of developing systolic dysfunction and heart failure.

Assess the patient's clinical status and LVEF prior to and regularly during treatment and adjust the MAVACAMTEN dose accordingly. New or worsening arrhythmia, dyspnea, chest pain, fatigue, palpitations, leg edema, or elevations in N-terminal pro-b-type natriuretic peptide (NT-proBNP) may be signs and symptoms of heart failure and should also prompt an evaluation of cardiac function.

Asymptomatic LVEF reduction, intercurrent illnesses, and arrhythmias require additional dosing considerations.

Initiation of MAVACAMTEN in patients with LVEF<55% is not recommended. Avoid concomitant use of MAVACAMTEN in patients on disopyramide, ranolazine, verapamil with a beta blocker, or diltiazem with a beta blocker as these medications and combinations were excluded from the clinical study of MAVACAMTEN in obstructive HCM (EXPLORER-HCM). Concomitant use of MAVACAMTEN with disopyramide in combination with verapamil or diltiazem has been associated with left ventricular systolic dysfunction and heart failure symptoms in patients with obstructive HCM.

5.2. CYP 450 Drug Interactions Leading to Heart Failure or Loss of Effectiveness

MAVACAMTEN is primarily metabolized by CYP2C19 and CYP3A4 enzymes. Concomitant use of MAVACAMTEN and drugs that interact with these enzymes may lead to life-threatening drug interactions such as heart failure or loss of effectiveness.

Advise patients of the potential for drug interactions, including with over-the-counter medications (such as omeprazole, esomeprazole, or cimetidine). Advise patients to inform their healthcare provider of all concomitant products prior to and during MAVACAMTEN treatment.

5.3. MAVACAMTEN REMS Program

MAVACAMTEN is only available through a restricted program called the MAVACAMTEN REMS Program because of the risk of heart failure due to systolic dysfunction.

Notable requirements of the MAVACAMTEN REMS Program include the following:
Prescribers must be certified by enrolling in the MAVACAMTEN REMS Program.
Patients must enroll in the MAVACAMTEN REMS Program and comply with ongoing monitoring requirements.
Pharmacies must be certified by enrolling in the MAVACAMTEN REMS Program and must only dispense to patients who are authorized to receive MAVACAMTEN.
Wholesalers and distributors must only distribute to certified pharmacies.

5.4. Embryo-Fetal Toxicity

MAVACAMTEN may cause fetal toxicity when administered to a pregnant female, based on findings in animal studies. Confirm absence of pregnancy in females of reproductive potential prior to treatment and advise patients to use effective contraception during treatment with MAVACAMTEN and for 4 months after the last dose. MAVACAMTEN may reduce the effectiveness of combined hormonal contraceptives (CHCs). Advise patients using CHCs to use an alternative contraceptive method that is not affected by CYP 450 enzyme induction or to add nonhormonal contraception.

Advise females of reproductive potential about the potential risk to the fetus with maternal exposure to MAVACAMTEN during pregnancy.

6. Adverse Reactions

The following adverse reaction is discussed in other sections of the labeling:
Heart failure

6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety of MAVACAMTEN was evaluated in EXPLORER-HCM, a Phase 3, double-blind, randomized, placebo-controlled trial. Of the 251 adults with obstructive HCM, 123 patients were treated with MAVACAMTEN 2.5-15 mg daily and 128 were treated with placebo. MAVACAMTEN-treated patients had a median duration of exposure of 30 weeks (range: 2-40 weeks).

Syncope (0.8%) was the only adverse drug reaction leading to discontinuation in patients receiving MAVACAMTEN.

Adverse reactions occurring in >5% of patients and more commonly on MAVACAMTEN than on placebo were dizziness (27% vs. 18%) and syncope (6% vs. 2%).

Effects on Systolic Function

In the EXPLORER-HCM trial, mean (SD) resting LVEF was 74% (6) at baseline in both treatment groups. Consistent with the mechanism of action of MAVACAMTEN, mean (SD) absolute change from baseline in LVEF was −4% (8) in the MAVACAMTEN group and 0% (7) in the placebo group over the 30-week treatment period. At Week 38, following an 8-week interruption of trial drug, mean LVEF was similar to baseline for both treatment groups. In the EXPLORER-HCM trial, 7 (6%) patients in the MAVACAMTEN group and 2 (2%) patients in the placebo group experienced reversible reductions in LVEF to <50% (median 48%: range 35-49%) while on treatment. In 3 of the 7 MAVACAMTEN patients and 1 of the 2 placebo patients, these reductions were asymptomatic. In all 7 patients treated with MAVACAMTEN, LVEF recovered following interruption of MAVACAMTEN.

7. Drug Interactions

7.1. Potential for Other Drugs to Affect Plasma Concentrations of MAVACAMTEN

Mavacamten is primarily metabolized by CYP2C19 and to a lesser extent by CYP3A4 and CYP2C9. Inducers and inhibitors of CYP2C19 and moderate to strong inhibitors or inducers of CYP3A4 may affect the exposures of mavacamten. (See Table 1)

TABLE 1
Established and Potentially Significant Pharmacokinetic Drug Interactions with MAVACAMTEN
Impact of Other Drugs on MAVACAMTEN
Moderate to Strong CYP2C19 Inhibitors or Strong CYP3A4 Inhibitors

| | |
|---|---|
| Clinical Impact | Concomitant use with a moderate to strong CYP2C19 or a strong |
| | CYP3A4 inhibitor increases mavacamten exposure, which may increase |
| | the risk of heart failure due to systolic dysfunction. |
| Prevention or Management | Concomitant use with a moderate to strong CYP2C19 inhibitor or a |
| | strong CYP3A4 inhibitor is contraindicated. |

Moderate to Strong CYP2C19 Inducers or Moderate to Strong CYP3A4 Inducers

| | |
|---|---|
| Clinical Impact | Concomitant use with a moderate to strong CYP2C19 inducer or a |
| | moderate to strong CYP3A4 inducer decreases mavacamten exposure, |
| | which may reduce MAVACAMTEN's efficacy. The risk of heart failure |
| | due to systolic dysfunction may increase with discontinuation of these |
| | inducers as the levels of induced enzyme normalizes. |
| Prevention or Management | Concomitant use of a moderate to strong CYP2C19 inducer or a moderate |
| | to strong CYP3A4 inducer is contraindicated. |

Weak CYP2C19 Inhibitors or Moderate CYP3A4 Inhibitors

| | |
|---|---|
| Clinical Impact | Concomitant use with a weak CYP2C19 inhibitor or a moderate CYP3A4 |
| | inhibitor increases mavacamten exposure, which may increase the risk of |
| | adverse drug reactions. |
| Prevention or Management | Initiate MAVACAMTEN at the recommended starting dosage of 5 mg |
| | orally once daily in patients who are on stable therapy with a weak |
| | CYP2C19 inhibitor or a moderate CYP3A4 inhibitor. |
| | Reduce dose of MAVACAMTEN by one level (i.e., 15 to 10 mg, 10 to 5 |
| | mg, or 5 to 2.5 mg) in patients who are on MAVACAMTEN treatment |
| | and intend to initiate a weak CYP2C19 inhibitor or a moderate CYP3A4 |
| | inhibitor. Avoid initiation of concomitant weak CYP2C19 and moderate |
| | CYP3A4 inhibitors in patients who are on stable treatment with 2.5 mg of |
| | MAVACAMTEN because a lower dose is not available. |

7.2. Potential for MAVACAMTEN to Affect Plasma Concentrations of Other Drugs

Mavacamten is an inducer of CYP3A4, CYP2C9, and CYP2C19. Concomitant use with CYP3A4, CYP2C19, or CYP2C9 substrates may reduce plasma concentration of these drugs. Closely monitor when MAVACAMTEN is used in combination with CYP3A4, CYP2C19, or CYP2C9 substrates where decreases in the plasma concentration of these drugs may reduce their activity.

Hormonal Contraceptives: Progestin and ethinyl estradiol are CYP3A4 substrates. Concomitant use of MAVACAMTEN may decrease exposures of ethinyl estradiol and progestin, which may lead to contraceptive failure or an increase in breakthrough bleeding. Advise patients to use a contraceptive method that is not affected by CYP 450 enzyme induction (e.g., intrauterine system) or add nonhormonal contraception (such as condoms) during concomitant use and for 4 months after the last dose of MAVACAMTEN.

7.3. Drugs that Reduce Cardiac Contractility

Expect additive negative inotropic effects of MAVACAMTEN and other drugs that reduce cardiac contractility. In the EXPLORER-HCM trial, 119 of 123 patients who received MAVACAMTEN received concomitant therapy with beta blockers (n=94), verapamil (n=19), or diltiazem (n=6).

Avoid concomitant use of MAVACAMTEN with disopyramide in combination with verapamil or diltiazem because such use has been associated with left ventricular systolic dysfunction and heart failure symptoms.

If concomitant therapy with a negative inotrope is initiated, or if the dose of a negative inotrope is increased, monitor LVEF closely until stable doses and clinical response have been achieved.

8. Use in Specific Populations

8.1. Pregnancy

Risk Summary

Based on animal data, MAVACAMTEN may cause fetal harm when administered to a pregnant female. There are no human data on the use of MAVACAMTEN during pregnancy to evaluate for a drug-associated risk of major birth defects, miscarriage, or other adverse maternal or fetal outcomes. The underlying maternal condition during pregnancy poses a risk to the mother and fetus. Advise pregnant females about the potential risk to the fetus with maternal exposure to MAVACAMTEN during pregnancy.

In animal embryo-fetal development studies, mavacamten-related decreases in mean fetal body weight, reductions in fetal ossification of bones, and increases in post-implantation loss (early and/or late resorptions) were observed in rats and increases in visceral and skeletal malformations were observed in both rabbits and rats at dose exposures similar to that achieved at the maximum recommended human dose (MRHD).

The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2% to 4% and 15% to 20%, respectively.

There is a pregnancy safety study for MAVACAMTEN. If MAVACAMTEN is administered during pregnancy, or if a patient becomes pregnant while receiving MAVACAMTEN or within 4 months after the last dose of MAVACAMTEN, healthcare providers should report MAVACAMTEN exposure.

Clinical Considerations

Disease-Associated Maternal and Embryo-Fetal Risk

Obstructive HCM in pregnancy has been associated with increased risk for preterm birth.

Data

Animal Data

When mavacamten was administered orally to pregnant rats (0.3 to 1.5 mg/kg/day) during the period of organogenesis, increases in post-implantation loss, decreases in mean fetal body weight, reductions in fetal ossification of bones, and fetal malformations (visceral and skeletal) were observed in the high dose group (1.5 mg/kg/day). Visceral malformations (heart malformation in fetuses, including one total situs inversus) and increased incidences of skeletal malformations (mainly fused sternebrae) were observed at a similar exposure as in humans at the MRHD. Plasma exposure (based on area under the concentration-time curve or AUC) at the no-effect dose for embryo-fetal development in rats is 0.3 times the exposure in humans at the MRHD.

When mavacamten was administered orally to pregnant rabbits (0.6 to 2.0 mg/kg/day) during the period of organogenesis, fetal malformations (visceral and skeletal) were increased at doses of 1.2 mg/kg/day and higher, with similar plasma exposure at 1.2 mg/kg/day as in humans at the MRHD. Visceral findings consisted of malformations of the great vessels (dilatation of pulmonary trunk and/or aortic arch). Skeletal malformations consisted of higher incidences of fused sternebrae at ≥1.2 mg/kg/day. Plasma exposure (AUC) at the no-effect dose for embryo-fetal development in rabbits is 0.4 times the exposure in humans at the MRHD.

In a pre/postnatal development study, mavacamten was administered orally to pregnant rats (0.3, to 1.5 mg/kg/day) from gestation Day 6 to lactation/post-partum Day 20. No adverse effects were observed in the dams or offspring exposed daily from before birth (in utero) through lactation. The no-observed-adverse-effect level (NOAEL) was 1.5 mg/kg/day (the highest dosage level tested), with similar exposure (AUC) as in humans at the MRHD.

8.2. Lactation

Risk Summary

The presence of mavacamten in human or animal milk, the drug's effects on the breastfed infant, and the effects on milk production are unknown. The developmental and health benefits of breastfeeding should be considered along with the mother's clinical need for MAVACAMTEN and any potential adverse effects on the breastfed child from MAVACAMTEN or from the underlying maternal condition.

8.3. Females and Males of Reproductive Potential

Based on animal data, MAVACAMTEN may cause fetal harm when administered to a pregnant female.

Pregnancy Testing

Confirm absence of pregnancy in females of reproductive potential prior to initiation of MAVACAMTEN.

Contraception

Females

Advise females of reproductive potential to use effective contraception during treatment with MAVACAMTEN and for 4 months after the last dose. Use of MAVACAMTEN may reduce the effectiveness of CHCs. Advise patients using CHCs to use an alternative contraceptive method or add nonhormonal contraception.

8.4. Pediatric Use

The safety and effectiveness of MAVACAMTEN have not been established in pediatric patients.

8.5. Geriatric Use

Clinical trials included 263 patients dosed with MAVACAMTEN, 95 of whom were 65 years of age or older (36.1%), and 17 of whom (6.5%) were age 75 years or older. Safety, effectiveness, and pharmacokinetics were similar between patients ≥65 years and younger patients.

8.6. Hepatic Impairment

No dosage adjustment is required in patients with mild (Child-Pugh A) to moderate (Child-Pugh B) hepatic impairment. Mavacamten exposure (AUC) increased up to 220% in patients with mild (Child-Pugh A) or moderate (Child-Pugh B) hepatic impairment compared to patients with normal hepatic function. However, no additional dose adjustment is required in patients with mild to moderate hepatic impairment with the recommended dose titration algorithm and monitoring plan. The effect of severe (Child-Pugh C) hepatic impairment is unknown.

10. Overdosage

Human experience of overdose with MAVACAMTEN is limited. MAVACAMTEN has been given as a single dose of up to 144 mg in patients with HCM. One subject administered a single dose of 144 mg experienced serious adverse events including vasovagal reaction, hypotension, and asystole, but the subject recovered. In healthy subjects, doses of up to 25 mg have been administered for up to 25 days, with 3 of 8 participants treated at the 25 mg dose level experiencing 20% or greater reductions in LVEF. An infant death was reported after accidental ingestion of three 15 mg capsules.

Systolic dysfunction is the most likely result of overdosage of MAVACAMTEN. Treatment of overdose with MAVACAMTEN consists of discontinuation of MAVACAMTEN treatment as well as medically supportive measures to maintain hemodynamic stability, including close monitoring of vital signs and LVEF and management of the clinical status of the patient. Overdose in humans can be life-threatening and result in asystole refractory to any medical intervention.

11. Description

MAVACAMTEN capsules for oral use contain mavacamten, a cardiac myosin inhibitor.

The chemical name of mavacamten is 3-(1-methylethyl)-6-[[(1S)-1-phenylethyl]amino]-2,4(1H,3H)-pyrimidinedione. The molecular formula is C₁₅H₁₉N₃O₂, and the molecular weight is 273.33 g/mol.

The structural formula of mavacamten is:

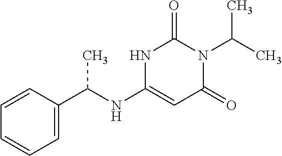 [SMILES: CC(C)n1c(=O)cc(N[C@@H](C)c2ccccc2)[nH]c1=O]

Mavacamten is a white to off-white powder that is practically insoluble in water and aqueous buffers at pH 2-10, sparingly soluble in methanol and ethanol, and freely soluble in DMSO and NMP.

MAVACAMTEN is supplied as immediate release Size 2 hard gelatin capsules, containing 2.5, 5, 10, or 15 mg of mavacamten per capsule as active ingredient and the following inactive ingredients: croscarmellose sodium, hypromellose, magnesium stearate (non-bovine), mannitol, and silicon dioxide. The capsule shell contains black edible ink, black iron oxide, gelatin, red iron oxide, titanium dioxide, and yellow iron oxide.

12. Clinical Pharmacology

12.1. Mechanism of Action

Mavacamten is an allosteric and reversible inhibitor selective for cardiac myosin. Mavacamten modulates the number of myosin heads that can enter “on actin” (power-generating) states, thus reducing the probability of force-producing (systolic) and residual (diastolic) cross-bridge formation. Excess myosin actin cross-bridge formation and dysregulation of the super-relaxed state are mechanistic hallmarks of HCM. Mavacamten shifts the overall myosin population towards an energy-sparing, recruitable, super-relaxed state. In HCM patients, myosin inhibition with mavacamten reduces dynamic LVOT obstruction and improves cardiac filling pressures.

12.2. Pharmacodynamics

Left Ventricular Ejection Fraction and Left Ventricular Outflow Tract Obstruction

In the EXPLORER-HCM trial, patients achieved reductions in mean resting and provoked (Valsalva) LVOT gradient by Week 4 which were sustained throughout the 30-week trial. At Week 30, the mean (SD) changes from baseline in resting and Valsalva LVOT gradients were −39 (29) mmHg and −49 (34) mmHg, respectively, for the MAVACAMTEN group and −6 (28) mmHg and −12 (31) mmHg, respectively, for the placebo group. The reductions in Valsalva LVOT gradient were accompanied by decreases in LVEF, generally within the normal range. Eight weeks after discontinuation of MAVACAMTEN, mean LVEF and Valsalva LVOT gradients were similar to baseline.

Cardiac Structure

In EXPLORER-HCM, echocardiographic measurements of cardiac structure showed a mean (SD) reduction from baseline at Week 30 in left ventricular mass index (LVMI) in the mavacamten group (−7.4 [17.8] g/m²) versus an increase in LVMI in the placebo group (8.9 [15.3] g/m²). There was also a mean (SD) reduction from baseline in left atrial volume index (LAVI) in the mavacamten group (−7.5 [7.8] mL/m²) versus no change in the placebo group (−0.1 [8.7] mL/m²). The clinical significance of these findings is unknown.

Cardiac Biomarkers

In the EXPLORER-HCM trial, reductions in a biomarker of cardiac wall stress, NT-proBNP, were observed by Week 4 and sustained through the end of treatment. At Week 30 compared with baseline, the reduction in NT-proBNP after mavacamten treatment was 80% greater than for placebo (proportion of geometric mean ratio between the two groups, 0.20 [95% CI: 0.17, 0.24]). The clinical significance of these findings is unknown.

Cardiac Electrophysiology

In healthy volunteers receiving multiple doses of MAVACAMTEN, a concentration-dependent increase in the QTc interval was observed at doses up to 25 mg once daily. No acute QTc changes have been observed at similar exposures during single-dose studies. The mechanism of the QT prolongation effect is not known.

A meta-analysis across clinical studies in HCM patients does not suggest clinically relevant increases in the QTc interval in the therapeutic exposure range. In HCM, the QT interval may be intrinsically prolonged due to the underlying disease, in association with ventricular pacing, or in association with drugs with potential for QT prolongation commonly used in the HCM population. The effect of coadministration of MAVACAMTEN with QT-prolonging drugs or in patients with potassium channel variants resulting in a long QT interval have not been characterized.

12.3. Pharmacokinetics

Mavacamten exposure increases generally dose proportionally after multiple once-daily doses of 1 mg to 15 mg. At the same dose level of MAVACAMTEN, 170% higher exposures of mavacamten are observed in patients with HCM compared to healthy subjects.

Absorption

Mavacamten has an estimated oral bioavailability of at least 85% and time to maximum concentration (Tmax) of 1 hour.

Effect of Food

No clinically significant differences in mavacamten pharmacokinetics were observed following its administration with a high fat meal. The Tmax was increased by 4 hours.

Distribution

Plasma protein binding of mavacamten is between 97 and 98%.

Elimination

Mavacamten has a variable terminal t1/2 that depends on CYP2C19 metabolic status. Mavacamten terminal half-life is 6-9 days in CYP2C19 normal metabolizers (NMs), which is prolonged in CYP2C19 poor metabolizers (PMs) to 23 days. Drug accumulation occurs with an accumulation ratio of about 2-fold for Cmax and about 7-fold for AUC in CYP2C19 NMs. The accumulation depends on the metabolism status for CYP2C19 with the largest accumulation observed in CYP2C19 PMs. At steady-state, the peak-to-trough plasma concentration ratio with once daily dosing is approximately 1.5.

Metabolism

Mavacamten is extensively metabolized, primarily through CYP2C19 (74%), CYP3A4 (18%), and CYP2C9 (8%).

Excretion

Following a single 25 mg dose of radiolabeled mavacamten, 7% of the dose was recovered in feces (1% unchanged) and 85% in urine (3% unchanged).

Specific Populations

No clinically significant differences in the pharmacokinetics of mavacamten were observed based on age (range: 18-82 years), sex, race, ethnicity, or mild (eGFR: 60 to 89 mL/min/1.73 m²) to moderate (eGFR: 30 to 59 mL/min/1.73 m²) renal impairment. The effects of severe (eGFR: 15 to 30 mL/min/1.73 m²) renal impairment and kidney failure (eGFR: <15 mL/min/1.73 m²; including patients on dialysis) are unknown.

Hepatic Impairment

Mavacamten exposures (AUC) increased up to 220% in patients with mild (Child-Pugh A) or moderate (Child-Pugh B) hepatic impairment. The effect of severe (Child-Pugh C) hepatic impairment is unknown.

Drug Interactions

Clinical Studies and Model-Informed Approaches

Weak CYP2C19 Inhibitors: Concomitant use of mavacamten (15 mg) with omeprazole (20 mg) once daily increased mavacamten AUCinf by 48% with no effect on Cmax in healthy CYP2C19 NMs and rapid metabolizers (RMs; e.g., *1/*17).

Moderate CYP3A4 Inhibitors: Concomitant use of mavacamten (25 mg) with verapamil sustained release (240 mg) increased mavacamten AUCinf by 15% and Cmax by 52% in intermediate metabolizers (IMs; e.g., *1/*2, *1/*3, *2/*17, *3/*17) and NMs of CYP2C19. Concomitant use of mavacamten with diltiazem in CYP2C19 PMs is predicted to increase mavacamten AUC0-24 h and Cmax up to 55% and 42%, respectively.

Strong CYP3A4 Inhibitors: Concomitant use of mavacamten (15 mg) with ketoconazole 400 mg once daily is predicted to increase mavacamten AUC0-24 and Cmax up to 130% and 90%, respectively.

Strong CYP2C19 and CYP3A4 Inducers: Concomitant use of mavacamten (a single 15 mg dose) with a strong CYP2C19 and CYP3A4 inducer (rifampin 600 mg daily dose) is predicted to decrease mavacamten AUC0-inf and Cmax by 87% and 22%, respectively in CYP2C19 NMs, and by 69% and 4%, respectively, in CYP2C19 PMs.

CYP3A4 Substrates: Concomitant use of a 16-day course of mavacamten (25 mg on days 1 and 2, followed by 15 mg for 14 days) resulted in a 13% and 7% decrease in midazolam AUCinf and Cmax, respectively, in healthy CYP2C19 NMs. Following coadministration of mavacamten once daily in HCM patients, midazolam AUCinf and Cmax are predicted to decrease by 21 to 64% and 13 to 48%, respectively, depending on the dose of mavacamten and CYP2C19 phenotype.

CYP2C8 Substrates: Concomitant use of mavacamten once daily in HCM patients is predicted to decrease AUC and Cmax of repaglinide, a CYP2C8 and CYP3A substrate, by 12 to 39%, depending on the dose of mavacamten and CYP2C19 phenotype.

CYP2C9 Substrates: Concomitant use of mavacamten once daily in HCM patients is predicted to decrease AUC and Cmax of tolbutamide, a CYP2C9 substrate, by 33 to 65%, depending on the dose of mavacamten and CYP2C19 phenotype.

CYP2C19 Substrates: Concomitant use of mavacamten once daily in HCM patients is predicted to decrease AUC and Cmax of omeprazole, a CYP2C19 substrate, by 48 to 67%, depending on the dose of mavacamten and CYP2C19 phenotype.

In Vitro Studies

CYP Enzymes: Mavacamten does not inhibit CYP1A2, CYP2B6, or CYP2C8. At clinically relevant concentrations, mavacamten is not an inhibitor of CYP2D6, CYP2C9, CYP2C19, or CYP3A4. Mavacamten is a CYP2B6 inducer.

Transporter Systems: Mavacamten does not inhibit P-gp, BCRP, BSEP, MATE1, MATE2-K, organic anion transporting polypeptides (OATPs), organic cation transporters (OCTs), or organic anion transporters (OATs).

12.5 Pharmacogenomics

Mavacamten AUCinf increased by 241% and Cmax increased by 47% in CYP2C19 poor metabolizers (PMs) compared to normal metabolizers (NMs) following a single dose of 15 mg mavacamten. Mean half-life is prolonged in CYP2C19 PMs compared to NMs (23 days vs. 6 to 9 days, respectively).

Polymorphic CYP2C19 is the main enzyme involved in the metabolism of MAVACAMTEN. An individual carrying two normal function alleles is a NM (e.g., *1/*1). An individual carrying two no function alleles is a PM (e.g., *2/*2, *2/*3, *3/*3).

The prevalence of CYP2C19 poor metabolizers differs depending on ancestry. Approximately 2% of individuals of European ancestry and 4% of individuals of African ancestry are PMs; the prevalence of PMs is higher in Asian populations (e.g., approximately 13% of East Asians).

13. Nonclinical Toxicology

13.1. Carcinogenesis, Mutagenesis, and Impairment of Fertility

Mavacamten was not genotoxic in a bacterial reverse mutation test (Ames test), a human in vitro lymphocyte clastogenicity assay, or a rat in vivo micronucleus assay.

There was no evidence of carcinogenicity seen in a 6-month rasH2 transgenic mouse study at mavacamten doses of up to 2.0 mg/kg/day in males and 3.0 mg/kg/day in females, which resulted in exposures (AUC) that were 1.8- and 3-fold in males and females, respectively, compared to AUC exposures in humans at the MRHD.

In reproductive toxicity studies, there was no evidence of effects of mavacamten on mating and fertility in male or female rats at doses up to 1.2 mg/kg/day, or on the viability and fertility of offspring of dams dosed up to 1.5 mg/kg/day. Plasma exposure (AUC) of mavacamten at the highest dose tested was the same as in humans at the MRHD.

13.2. Animal Toxicology and/or Pharmacology

The safety of mavacamten has been evaluated in rats and dogs at multiple dose levels (0.06 to 10 mg/kg/day) orally. Noted toxicities, including echocardiographic findings, reduction in systolic function, cardiac dilation, and death, as well as increased heart weights in rats, were consistent with mavacamten's mechanism of action and primary pharmacological activity. Other findings included cardiac osseous metaplasia in rats and QTc prolongation in dogs. Plasma exposures (AUC) at the NOAEL in rats and dogs were 0.1 and 0.3 times, respectively, human exposure (AUC) at the MRHD.

14. Clinical Studies

Obstructive Hypertrophic Cardiomyopathy

The efficacy of MAVACAMTEN was evaluated in EXPLORER-HCM (NCT-03470545) a Phase 3, double-blind, randomized, placebo-controlled, multicenter, international, parallel-group trial in 251 adults with symptomatic NYHA class II and III obstructive HCM, LVEF≥55%, and Valsalva LVOT peak gradient ≥50 mmHg at rest or with provocation.

Patients on dual therapy with beta blocker and calcium channel blocker treatment or monotherapy with disopyramide or ranolazine were excluded. Patients with a known infiltrative or storage disorder causing cardiac hypertrophy that mimicked obstructive HCM, such as Fabry disease, amyloidosis, or Noonan syndrome with left ventricular hypertrophy, were also excluded.

Patients were randomized in a 1:1 ratio to receive either a starting dose of 5 mg of MAVACAMTEN or placebo once daily for 30 weeks. Treatment assignment was stratified by baseline NYHA functional class, baseline use of beta blockers, and type of ergometer (treadmill or exercise bicycle).

Groups were well matched with respect to age (mean 59 years), BMI (mean 30 kg/m²), heart rate (mean 62 bpm), blood pressure (mean 128/76 mmHg), and race (90% Caucasian). Males comprised 54% of the MAVACAMTEN group and 65% of the placebo group.

At baseline, approximately 73% of the randomized patients were NYHA class II and 27% were NYHA class III. The mean LVEF was 74%, and the mean Valsalva LVOT gradient was 73 mmHg. About 10% had prior septal reduction therapy, 75% were on beta blockers, 17% were on calcium channel blockers, and 14% had a history of atrial fibrillation.

All patients were initiated on MAVACAMTEN 5 mg (or matching placebo) once daily, and the dose was periodically adjusted to optimize patient response (decrease in LVOT gradient with Valsalva maneuver) and maintain LVEF≥50%. The dose was also informed by plasma concentrations of MAVACAMTEN.

In the MAVACAMTEN group, at the end of treatment, 49% of patients were receiving the 5 mg dose, 33% were receiving the 10 mg dose, and 11% were receiving the 15 mg dose. Three patients temporarily interrupted their dose due to LVEF<50%, of whom two resumed treatment at the same dose and one had the dose reduced from 10 mg to 5 mg.

Primary Endpoint

The primary composite functional endpoint, assessed at 30 weeks, was defined as the proportion of patients who achieved either improvement of mixed venous oxygen tension (pVO2) by ≥1.5 mL/kg/min plus improvement in NYHA class by at least 1 or improvement of pVO2 by ≥3.0 mL/kg/min plus no worsening in NYHA class.

A greater proportion of patients met the primary endpoint at Week 30 in the MAVACAMTEN group compared to the placebo group (37% vs. 17%, respectively, p=0.0005; see Table 2).

TABLE 2
Primary Endpoint at 30 Weeks

| | MAVACAMTEN | Placebo | Difference | |
|---|---|---|---|---|
| | N = 123 | N = 128 | (95% CI) | p-value |
| Total responders | 45 (37%) | 22 (17%) | 19% (9, 30) | 0.0005 |
| Change from baseline | 41 (33%) | 18 (14%) | 19% (9, 30) | |
| pVO₂ ≥1.5 mL/kg/min and | | | | |
| decreased NYHA | | | | |
| Change from baseline | 29 (23%) | 14 (11%) | 13% (3, 22) | |
| pVO₂ ≥3 mL/kg/min and | | | | |
| NYHA not increased | | | | |

A range of demographic characteristics, baseline disease characteristics, and baseline concomitant medications were examined for their influence on outcomes. Results of the primary analysis consistently favored MAVACAMTEN across all subgroups analyzed (FIG. 17).

FIG. 17 shows a Subgroup Analysis of the Primary Composite Functional Endpoint.

The dashed vertical line represents the overall treatment effect and the solid vertical line (no effect) indicates no difference between treatment groups. Note: The figure presents effects in various subgroups, all of which are baseline characteristics. The 95% confidence limits that are shown do not take into account the number of comparisons made and may not reflect the effect of a particular factor after adjustment for all other factors. Apparent homogeneity or heterogeneity among groups should not be over-interpreted.

Although the benefit of mavacamten was smaller in patients on background beta blocker therapy compared to those who were not (attenuated improvement in pVO2), analyses of other secondary endpoints (symptoms, LVOT gradient) suggest that patients might benefit from mavacamten treatment regardless of beta blocker use.

Secondary Endpoints

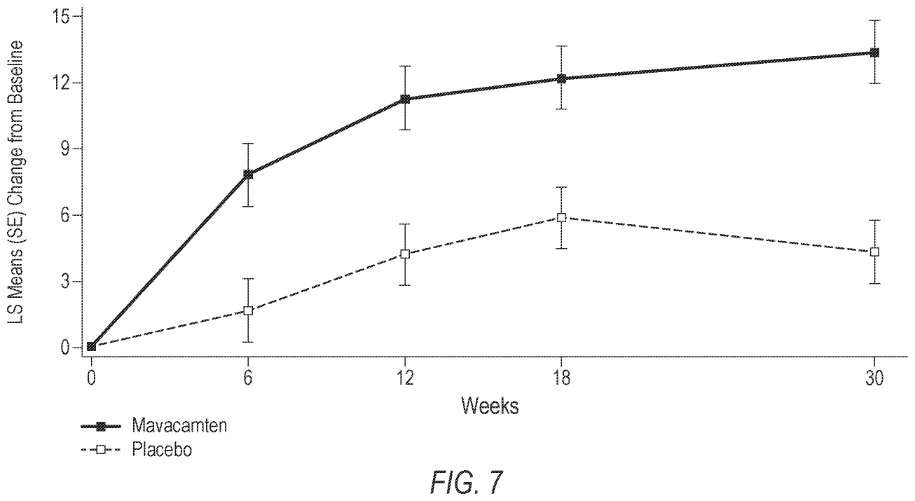

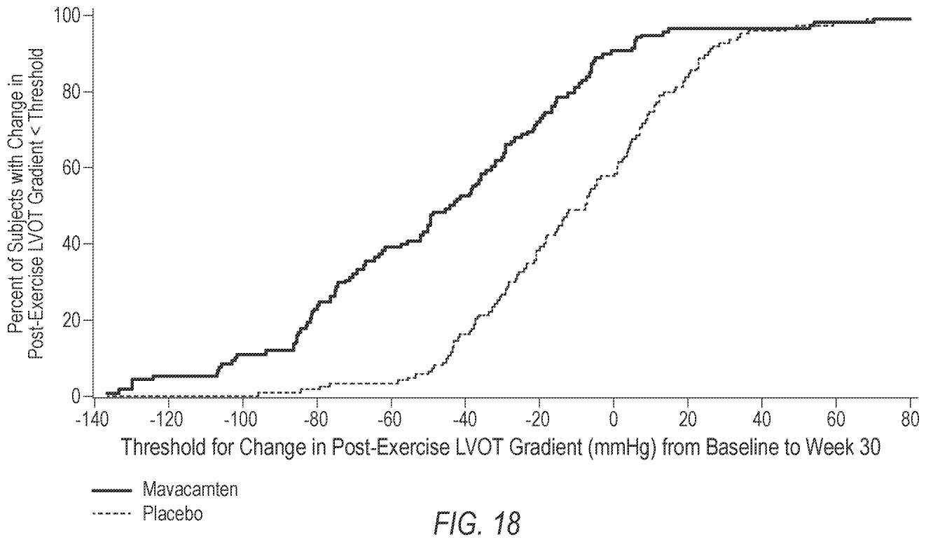

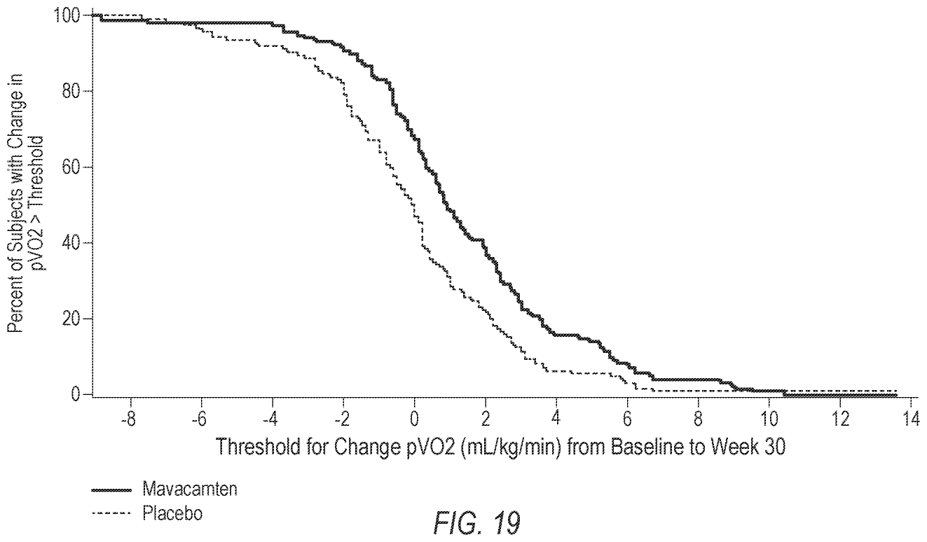

The treatment effects of MAVACAMTEN on LVOT obstruction, functional capacity, and health status were assessed by change from baseline through Week 30 in post-exercise LVOT peak gradient, change in pVO2, proportion of patients with improvement in NYHA class, Kansas City Cardiomyopathy Questionnaire-23 (KCCQ-23) Clinical Summary Score (CSS), and Hypertrophic Cardiomyopathy Symptom Questionnaire (HCMSQ) Shortness of Breath (SoB) domain score. At Week 30, patients receiving MAVACAMTEN had greater improvement compared to the placebo group across all secondary endpoints (Table 3, FIG. 18, FIG. 19, Table 4, and FIGS. 7-10).

TABLE 3
Change from Baseline to Week 30 in Post-Exercise
LVOT Gradient, pVO2, and NYHA Class

| | MAVACAMTEN | Placebo | | |
|---|---|---|---|---|
| | N = 123 | N = 128 | Difference (95% CI) | p-value |
| Post-Exercise LVOT | −47 (40) | −10 (30) | −35 (−43, −28) | <0.0001 |
| gradient (mmHg), mean | | | | |
| (SD) | | | | |
| pVO₂ (mL/kg/min), mean | 1.4 (3.1) | −0.1 (3.0) | 1.4 (0.6, 2.1) | <0.0006 |
| (SD) | | | | |
| Number (%) with NYHA | 80 (65%) | 40 (31%) | 34% (22%, 45%) | <0.0001 |
| Class improved ≥1 | | | | |

FIG. 18 shows the Cumulative Distribution of Change from Baseline to Week 30 in LVOT Peak Gradient

FIG. 19 shows the Cumulative Distribution of Change from Baseline to Week 30 in pVO2

TABLE 4
Change from Baseline to Week 30 in KCCQ-23 CSS and HCMSQ SoB Domain

| | | | | | Difference, |
|---|---|---|---|---|---|
| | | | Change from Baseline | | LS Mean |
| | Baseline, Mean (SD) | | to Week 30, Mean (SD) | | (95% CI) and |
| | MAVACAMTEN | Placebo | MAVACAMTEN | Placebo | p-value |
| KCCQ-23 CSS^{†} | n = 99 | n = 97 | 14 (14) | 4 (14) | 9 (5, 13) |
| | 71 (16) | 71 (19) | | | p < 0.0001 |
| KCCQ-23 TSS | 71 (17) | 69 (22) | 12 (15) | 5 (16) | |
| KCCQ-23 PL | 70 (18) | 72 (19) | 15 (17) | 4 (15) | |
| HCMSQ SoB^{‡} | n = 108 | n = 109 | −3 (3) | −1 (2) | −2 (−2, −1) |
| | 5 (3) | 5 (3) | | | p < 0.0001 |

^{†}The KCCQ-23 CSS is derived from the Total Symptom Score (TSS) and the Physical Limitations (PL) score of the KCCQ-23. The CSS ranges from 0 to 100 with higher scores representing less severe symptoms and/or physical limitations.
^{‡}The HCMSQ SoB domain score measures the frequency and severity of shortness of breath. The HCMSQ SoB domain score ranges from 0 to 18 with lower scores representing less shortness of breath.

Missing data were not imputed to summarize the baseline and change from baseline to Week 30 values. Difference in mean change from baseline between treatment groups was estimated using a mixed model for repeated measures.

FIG. 7 shows the time course for changes in KCCQ-23 CSS. FIG. 8 shows the distribution of changes from baseline to Week 30 for KCCQ-23 CSS.

The figure displays the cumulative percentage of patients achieving a certain level of response.

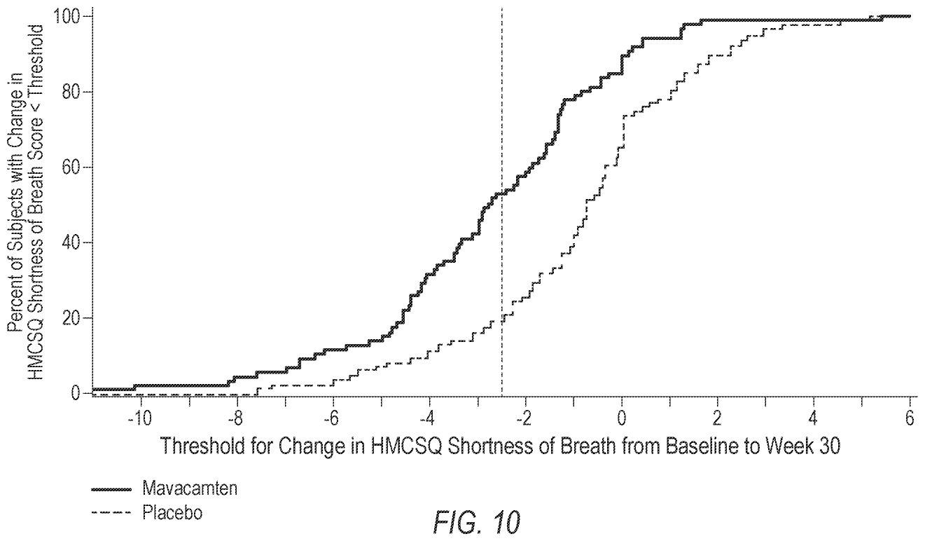

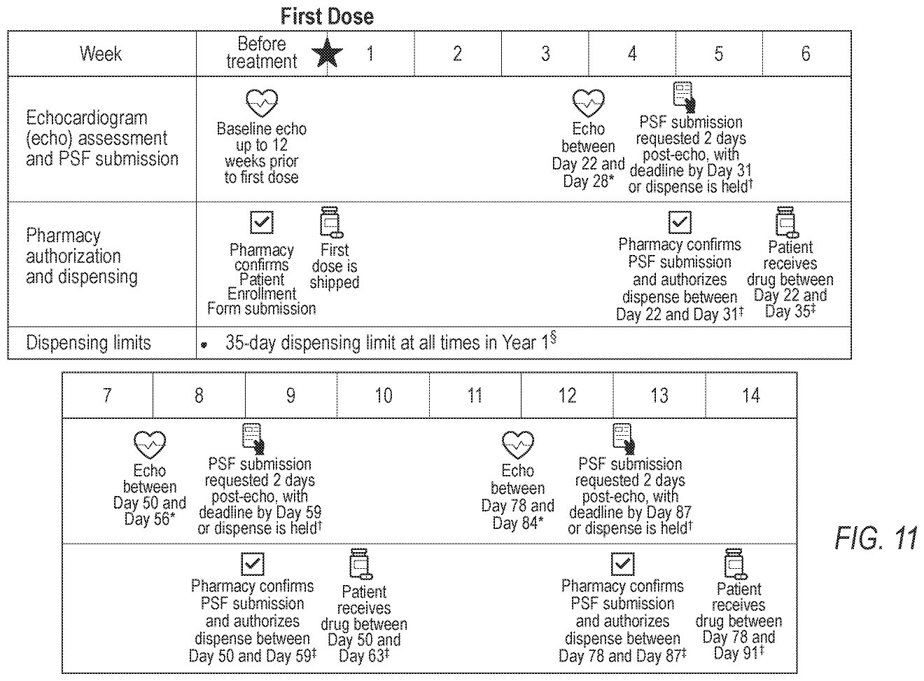

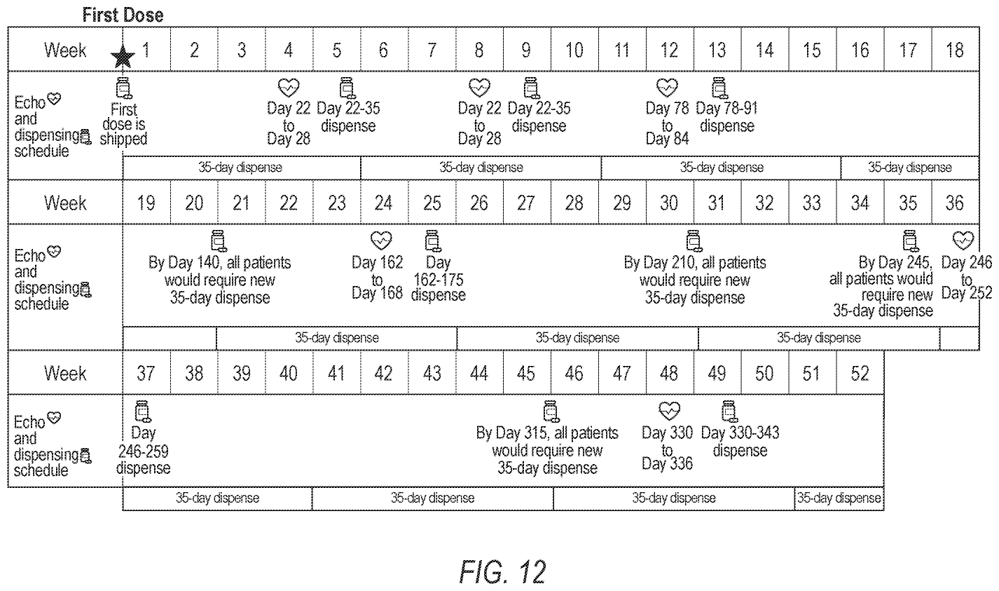

FIG. 9 shows the time course for changes in HCMSQ SoB. FIG. 10 shows the distribution of changes from baseline to Week 30 for HCMSQ SoB.

16. How Supplied/Storage and Handling

MAVACAMTEN is supplied as immediate release Size 2 hard gelatin capsules containing 2.5, 5, 10, or 15 mg of mavacamten. White opaque capsule bodies are imprinted with “Maya”, and the opaque cap is imprinted with the strength. The capsule contains white to off-white powder. MAVACAMTEN capsules are available in bottles of 30 capsules, as listed in the table below:

| Strength | Capsule Cap | NDC Number |
|---|---|---|
| 2.5 mg | Light purple | 73625-111-11 |
| 5 mg | Yellow | 73625-112-11 |
| 10 mg | Pink | 73625-113-11 |
| 15 mg | Gray | 73625-114-11 |

Storage

Store at 20° C. to 25° C. (68° F. to 77° F.), excursions permitted between 15° C. and 30° C. (between 59° F. and 86° F.) [see USP Controlled Room Temperature].

17. Patient Counseling Information

Advise the patient and/or caregiver to read the FDA-approved patient labeling (Medication Guide).

Heart Failure

Inform patients that cardiac function monitoring must be performed using echocardiography to monitor for heart failure. Advise patients to report any signs or symptoms of heart failure immediately to their healthcare provider.

Drug Interactions

Advise patients to inform their healthcare providers of all concomitant products, including over-the-counter medications (such as omeprazole, esomeprazole, or cimetidine) and supplements, prior to and during MAVACAMTEN treatment.

MAVACAMTEN REMS Program

MAVACAMTEN is available only through a restricted program called the MAVACAMTEN REMS Program. Inform the patient of the following notable requirements:
Patients must enroll in the program and comply with ongoing monitoring requirements.

MAVACAMTEN is only prescribed by certified healthcare providers and only dispensed from certified pharmacies participating in the program. Provide patients with the telephone number and website for information on how to obtain the product.

Embryo-Fetal Toxicity

Advise pregnant females and females of reproductive potential of the potential risk to a fetus. Advise females of reproductive potential to inform their healthcare provider of a known or suspected pregnancy.

Advise females of reproductive potential to use effective contraception during treatment with MAVACAMTEN and for 4 months after the last dose. Advise patients using CHCs to use an alternative contraceptive method or add nonhormonal contraception because MAVACAMTEN may decrease the efficacy of CHCs.

Advise females who are exposed to MAVACAMTEN during pregnancy that there is a pregnancy safety study that monitors pregnancy outcomes.

Instructions for Taking MAVACAMTEN

MAVACAMTEN capsules should be swallowed whole. Advise patients that if they miss a dose of MAVACAMTEN, to take the dose as soon as possible that day and the next scheduled dose should be taken at the usual time the following day. The patient should not take two doses in the same day.

### Example 2. Simulation of Dosing Regimens for Safety and Efficacy

Mavacamten has substantial pharmacokinetic (PK) variability, the largest contributor being CYP2C19 phenotype. Beyond phenotype, and after incorporation of all PK covariates, a moderate inter-individual variability remains. As a result of the PK variability, there is a need to achieve an ideal balance of reducing the Valsalva left ventricular outflow tract gradient (VLVOT) while maintaining the patient's left ventricular ejection fraction (LVEF), and an echocardiogram (ECHO) based titration regimen was developed. Modeling and simulation analyses have been undertaken to further evaluate dose titration by CYP2C19 phenotype.

In addition, an exposure-response simulation-based approach was utilized to investigate multiple titration regimens using an equal distribution of CYP2C19 phenotype to allow for additional sensitivity in assessing the performance of the titration regimen for each phenotype. The evaluations included genotype specific dosing and titration, adjusted starting dose of 2.5 mg and additional monitoring intervals. For each posology investigated, the key objectives remained consistent: to limit the percent (%) of simulated patients experiencing LVEF<50%, while gradually up-titrating them to achieve VLVOT<30 mmHg. This Example outlines the evaluation of 3 titration regimens, described below.

The methods used to conduct the simulations are described as follows. To investigate the regimens described, 5000 virtual oHCM patients were simulated, with approximately 1000 simulated patients from each of the following 5 CYP2C19 phenotypes: poor metabolizers (PM), intermediate metabolizers (IM), normal metabolizers (NM), rapid metabolizers (RM), and ultra-rapid metabolizers (UM). To construct distributions of simulated patient covariates, sets of covariates were chosen from the set of active-treatment subjects in Studies MYK-461-005 (EXPLORER-HCM, NCT03470545) and MYK-461-007 (MAVA-LTE, for subjects with oHCM, NCT03723655), stratified by phenotype.

The same 5000 simulated patients were utilized in the simulation of each regimen to allow for a robust comparison of the performance of each simulated regimen by metabolizer status, and to control for patient covariates or residual variability at each week contributing to differences between regimens.

Each regimen was simulated for 104 weeks, with the simulation code designed to mimic, as closely as possible, the regimen of interest. For any patient that required a dose interruption, a visit was added (if it was not part of the regimen) 4 weeks later, to assess if the simulated patient met the criteria for restart of therapy. If they did not meet criteria, they remained off-therapy for an additional 4 weeks and were “forced” to restart 4 weeks later (after a total of 8 weeks) if no visit was planned (i.e., the simulation code implemented a restart without a visit 4 weeks later, which is a simplification of the clinical implementation of the titration regimen which states restarting after 4-6 weeks of dose interruption). Patients were restarted at the next lower dose strength available and if discontinued on 2.5 mg, they were restarted at the same 2.5 mg dose.

The simulations described below in each section have been arranged to provide the following key items:
A table showing the percentage of patients meeting LVEF≤50% stratified by CYP2C19 metabolizer status/phenotype at selected time points over 104 weeks A table showing the percentage of patients meeting LVOT≤30 mmHg stratified by
CYP2C19 metabolizer status/phenotype at the same time points over 104 weeks
A table showing the percentage of patients with mavacamten plasma concentration >1000 ng/mL stratified by CYP2C19 metabolizer status/phenotype at the same time points over 104 weeks
A figure showing each of the key safety and efficacy thresholds (percentage of patients who have LVEF≤50%, LVOT≤30 mmHg, mavacamten plasma concentration >700 ng/mL, and mavacamten plasma concentration >1000 ng/mL) stratified by CYP2C19 metabolizer status/phenotype
The dose distribution in each CYP2C19 metabolizer status/phenotype at Week 104 stratified by CYP2C19 metabolizer status/phenotype.

Regimen #1: First Proposed Titration Regimen Using Uniform CYP2C19 Stratification

For this scenario, the proposed ECHO-based posology was as follows:
Each patient begins therapy with a 5 mg once daily dose of mavacamten.
All patients return for a visit at Week 4 to assess if they meet criteria for dose interruption (LVEF<50%) or down-titration VLVOT<20 mmHg.
All patients return for a visit at Week 12 for an up-titration opportunity (for patients with LVEF≥55% and VLVOT≥30 mmHg).
Patients who had a dose interruption at Week 4 return 4 weeks later to assess if they meet the LVEF criterion to restart therapy at the next lower dose (if LVEF recovered ≥50%). This criterion is applied at every visit.
Patients who were up-titrated at Week 12 return 4 weeks later to assess LVEF and LVOT gradient. After this 4-week follow up, the next visit is scheduled 12 weeks later (Week 28) for the next opportunity for an up-titration, and every 12 weeks thereafter (unless there is a dose up-titration or interruption, which would result in an additional 4-week follow-up visit).
Subsequent to the Week 12 visit, patients with no dose change are evaluated at a visit 12 weeks later and every 12 weeks thereafter until Week 52, and every 24 weeks from Week 52 to 104.
After Week 40, all patients follow the same visit schedule as follows: a visit 12 weeks later at Week 52, and every 24 weeks thereafter. To account for 4-week follow up visits which are clinically intended only for patients with up-titration or dose interruption, all simulated patients are assessed 4 weeks after the quarterly (12 weeks) or biannual (24 weeks) visit.
Up-titrations cannot occur within 12 weeks of one another.
For simplification in the code, a ‘force restart’ was implemented 8 weeks after dose interruption if patients did not meet the criteria for LVEF≥50% at the coded 4-week follow-up visit. This simplification is implemented in four instances, once at Week 20 and 3 times between Weeks 40-104.

Table 5 shows the percentage of patients attaining an LVEF≤50% across selected time points of the 104-week period stratified by phenotype. Generally, the highest percentage of patients with LVEF≤50% was based on phenotype status, with the greatest occurrence in PMs (10.2% at Week 24) and the lowest occurrence in UMs (<2% at all time points). This relationship was also seen with LVOT, with the highest percentage of patients meeting LVOT≤30 mmHg in PMs and the lowest percentage in Ums (Table 6) In all phenotypes, >60% of patients met LVOT≤30 mmHg after Week 18.

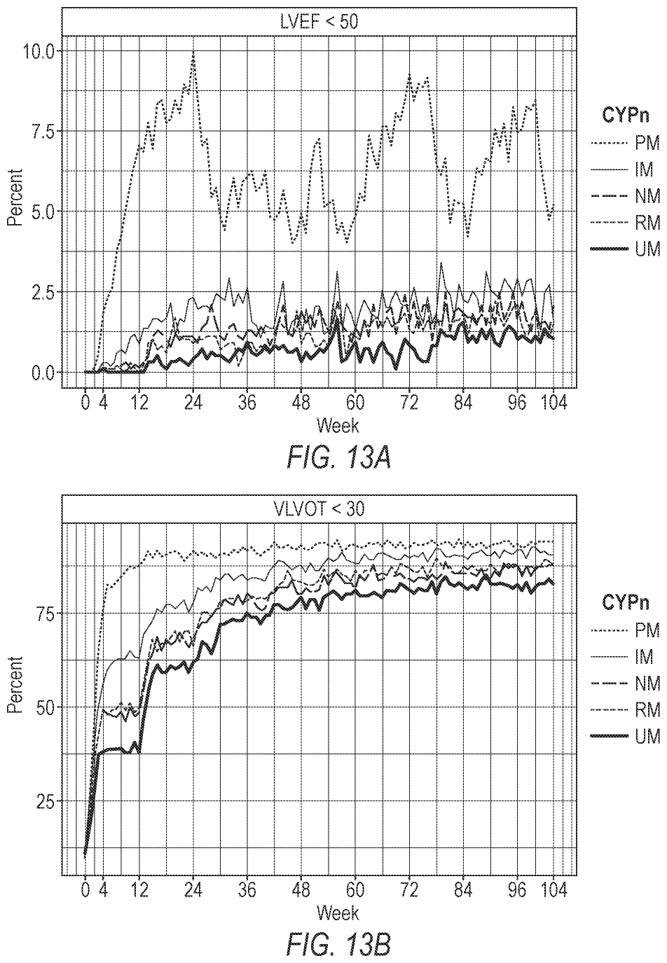

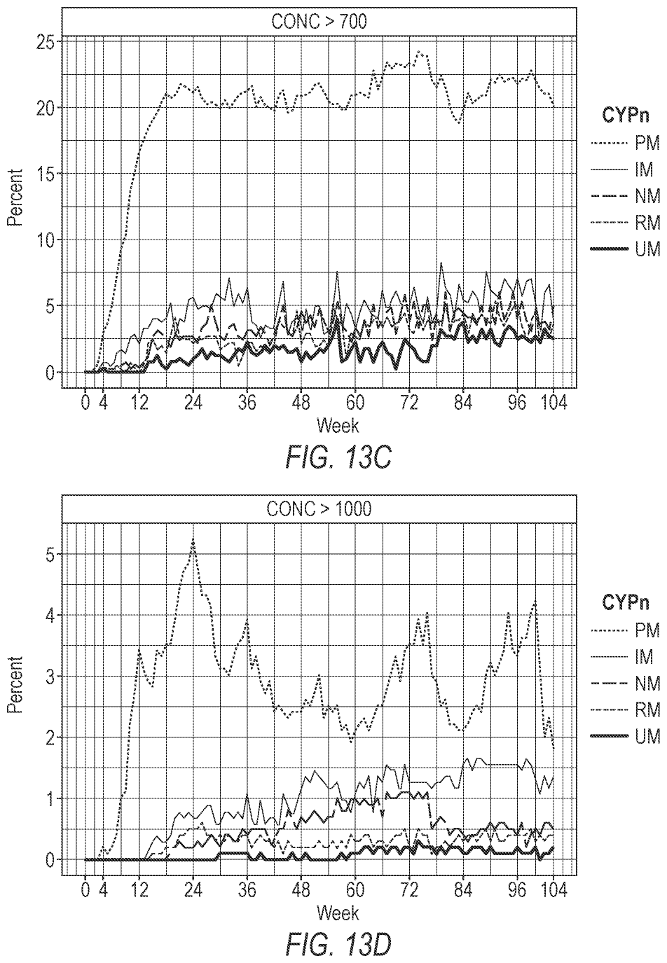

Table 7 shows that the percentage of patients attaining mavacamten plasma concentration >1000 ng/mL was generally low, with no more than 4% exceeding this threshold across the phenotypes. FIG. 13 depicts the key safety and efficacy thresholds including mavacamten plasma concentration >700 ng/mL over the 104-week period. The dose distribution depicted in Table 8 shows that there is a wide spread of doses across all phenotype assigned in the simulated patients at Week 104, indicating no single dose strength is appropriate for all patients using the proposed posology.

TABLE 5
Regimen #1: Percent of Patients Attaining
LVEF ≤50% Across Time Points During the
104-Week Dosing Period Stratified by Phenotype

| Week | PM | IM | NM | RM | UM |
|---|---|---|---|---|---|
| 4 | 1.81 | 0.292 | 0.0993 | 0.1 | 0.103 |
| 8 | 4.23 | 0.68 | 0.199 | 0.1 | 0 |
| 10 | 5.84 | 1.07 | 0.0993 | 0.301 | 0 |
| 12 | 7.04 | 0.875 | 0.0993 | 0 | 0 |
| 18 | 7.75 | 1.65 | 1.09 | 0.602 | 0.103 |
| 24 | 9.96 | 2.33 | 1.09 | 0.903 | 0.411 |
| 30 | 4.83 | 2.33 | 1.09 | 0.702 | 0.514 |
| 36 | 6.04 | 2.62 | 1.29 | 0.903 | 0.925 |
| 48 | 4.93 | 1.26 | 1.89 | 0.903 | 0.308 |
| 72 | 9.26 | 2.04 | 1.59 | 1.3 | 0.822 |
| 104 | 5.23 | 1.46 | 2.09 | 1.5 | 1.03 |

TABLE 6
Regimen #1: Percent of Patients Attaining
LVOT ≤30 mmHg Across Time Points During the
104-Week Dosing Period Stratified by Phenotype

| Week | PM | IM | NM | RM | UM |
|---|---|---|---|---|---|
| 4 | 75.6 | 55.8 | 48.2 | 49.2 | 38 |
| 8 | 84.7 | 62.8 | 48.6 | 51.2 | 39 |
| 10 | 87.3 | 65.1 | 49.8 | 51.1 | 38 |
| 12 | 87.5 | 63 | 48.3 | 49.6 | 38 |
| 18 | 90.5 | 77.2 | 67.8 | 66.7 | 59.3 |
| 24 | 91.4 | 77.6 | 67.1 | 66.2 | 61.9 |
| 30 | 90.6 | 83.7 | 76.6 | 78.1 | 71.9 |
| 36 | 91.8 | 83.8 | 80.1 | 78 | 75 |
| 48 | 93.1 | 87.7 | 79.6 | 83.1 | 79.2 |
| 72 | 92.7 | 89.7 | 84.2 | 87 | 81.1 |
| 104 | 94.1 | 90.4 | 88 | 87.9 | 82.7 |

TABLE 7
Regimen #1: Percent of Patients Attaining Mavacamten
Plasma Concentration >1000 ng/mL Across Time Points
During the 104-Week Dosing Period Stratified by Phenotype

| Week | PM | IM | NM | RM | UM |
|---|---|---|---|---|---|
| 4 | 0.201 | 0 | 0 | 0 | 0 |
| 8 | 1.01 | 0 | 0 | 0 | 0 |
| 10 | 2.21 | 0 | 0 | 0 | 0 |
| 12 | 3.42 | 0 | 0 | 0 | 0 |
| 18 | 3.52 | 0.292 | 0 | 0.201 | 0 |
| 24 | 5.23 | 0.68 | 0.199 | 0.502 | 0 |
| 30 | 3.12 | 0.583 | 0.199 | 0.401 | 0.103 |
| 36 | 3.92 | 1.07 | 0.397 | 0.401 | 0.103 |
| 48 | 2.41 | 1.17 | 0.695 | 0.201 | 0 |
| 72 | 3.52 | 1.26 | 1.09 | 0.201 | 0.206 |
| 104 | 1.81 | 1.36 | 0.497 | 0.401 | 0.206 |

FIG. 13 (A-D) shows the Time-Course of Percent of Patients with LVEF≤50%, VLVOT≤30 mmHg, Mavacamten Plasma Concentration >700 ng/mL, and Mavacamten Plasma Concentration >1000 ng/mL (104 Week Treatment Duration) under Regimen #1.

TABLE 8
Regimen #1: Dose Distribution
at Week 104 Stratified by Phenotype

| CYP | 0 mg | 2.5 mg | 5 mg | 10 mg | 15 mg |
|---|---|---|---|---|---|
| PM | 8.45 | 50.70 | 32.90 | 7.14 | 0.80 |
| IM | 2.04 | 15.35 | 36.15 | 33.62 | 12.83 |
| NM | 1.99 | 6.45 | 26.91 | 37.04 | 27.61 |
| RM | 1.91 | 6.92 | 26.88 | 37.71 | 26.58 |
| UM | 1.13 | 3.19 | 17.06 | 35.56 | 43.06 |

Regimen #2: Second Proposed Posology with Additional ECHO Monitoring

This posology maintains some similarities to the originally proposed posology in that it represents one “unified” posology for all patients with the same starting dose (5 mg), LVEF criteria for up- and down-titration, and VLVOT criteria for up- and down-titration. The Regimen #2 posology is consistent the posology in Example 1, and FIGS. 4-6. The key changes to the posology compared to Regimen #1 include an additional visit at Week 8 for all patients with opportunity for down-titration, opportunity for permanent discontinuation for patients who were unable to tolerate the lowest dose of mavacamten (as defined by patients who achieve LVEF<50% twice during treatment with a 2.5 mg dose) and continuation of every 3 month monitoring intervals into the second year of treatment. At the Week 8 visit, an additional down-titration opportunity for patients still on 5 mg was implemented for patients who were not down-titrated at Week 4 and met VLVOT<20 mmHg at criteria. The simulation implementation remained consistent with the methods outlined above. In addition, after the first up-titration opportunity at week 12, all patient visits were synchronized to every 12 weeks, rather than 12-weeks post their previous visit. Visits every 12 weeks were maintained for the second year rather than visits every 24 weeks.

Table 9 shows the percentage of patients attaining an LVEF<=50% across selected time points of the 104-week period stratified by phenotype. Generally, the highest percentage of patients with LVEF≤50% was based on phenotype status, with the greatest occurrence in PMs (4.2% at Week 8) and the lowest occurrence in UMs (≤1% at all time points). This relationship was also seen with LVOT, with the highest percentage of patients meeting LVOT≤30 mmHg in PMs and the lowest percentage in UMs (Table 10). In all phenotypes except for UM, >60% of patients met LVOT≤30 mmHg after Week 18.

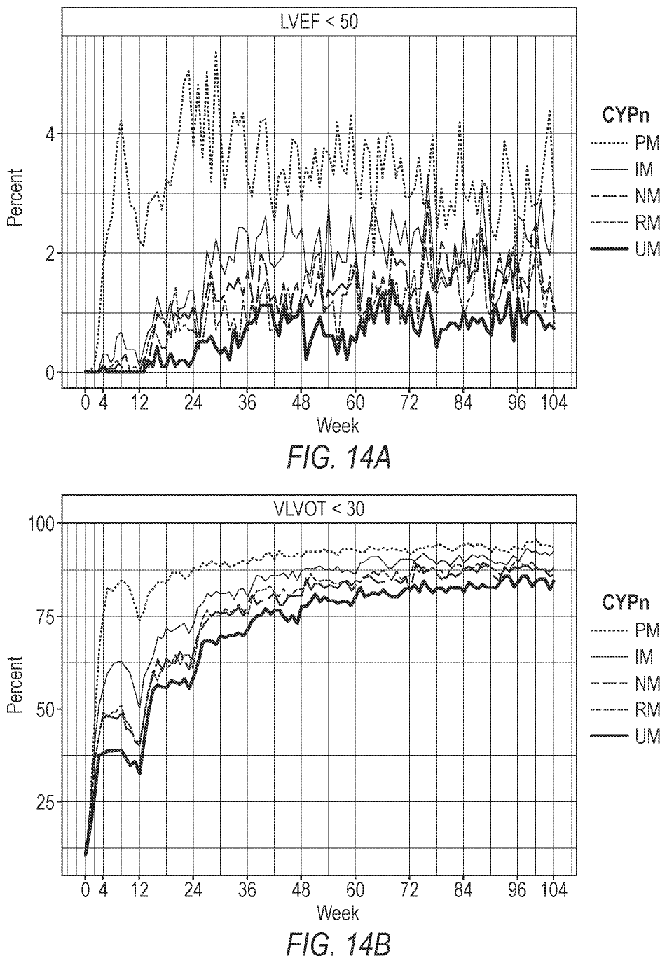

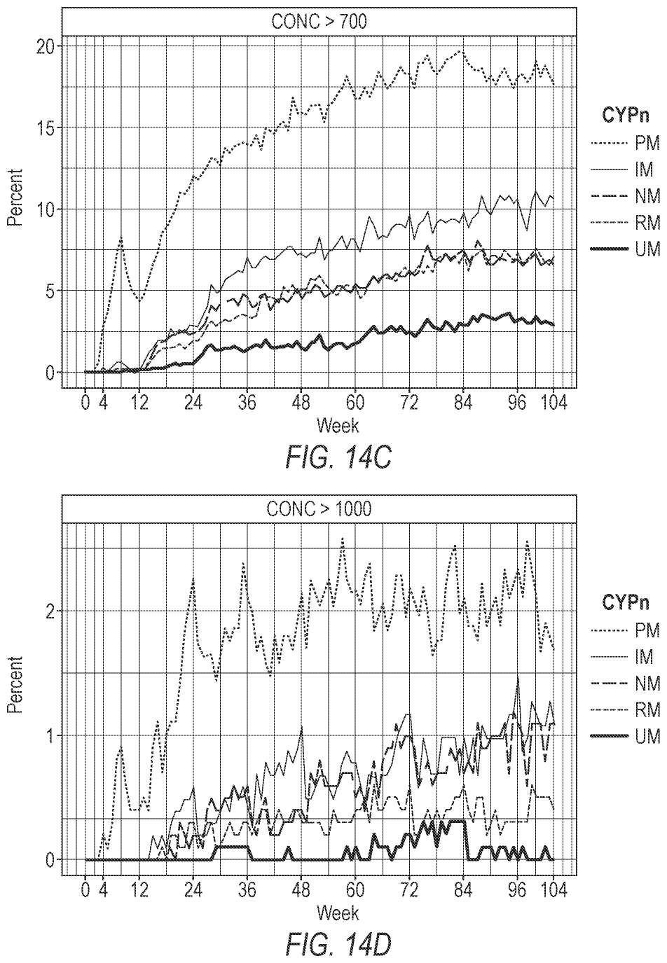

Table 11 shows that the percentage of patients attaining mavacamten plasma concentration >1000 ng/mL was generally low, with no more than ˜2% exceeding this threshold across the phenotypes. FIG. 14 depicts the key safety and efficacy thresholds including mavacamten plasma concentration >700 ng/mL over the 104-week period. The dose distribution depicted in Table 12 shows that there continues to be a wide spread of doses across all phenotype, and that the eventual distribution is relatively consistent to the original posology, with the exception of ˜10% of PMs who get permanently discontinued during the course of treatment. This criteria appears to have minimal impact on all other metabolizer phenotypes and allows for an improved benefit/risk overall, while appropriately identifying those patients who may not be able to tolerate the lowest available dose strength. According to the simulated dose distribution below, this reflects about 10% of the PM population and 0.3% of the IM population. Considering the CYP2C19 phenotype distribution in the EU population, this criteria would exclude/permanently discontinue <0.5% of patients overall.

TABLE 9
Regimen #2: Percent of Patients Attaining
LVEF ≤50% Across Time PointsDuring the
104-Week Dosing Period Stratified by Phenotype

| Week | PM | IM | NM | RM | UM |
|---|---|---|---|---|---|
| 4 | 1.81 | 0.292 | 0.0993 | 0.1 | 0.103 |
| 8 | 4.23 | 0.68 | 0.199 | 0.1 | 0 |
| 10 | 2.82 | 0.389 | 0 | 0 | 0 |
| 12 | 2.21 | 0.0972 | 0 | 0 | 0 |
| 18 | 3.23 | 0.875 | 0.794 | 0.401 | 0.103 |
| 24 | 3.79 | 1.36 | 1.09 | 0.702 | 0.206 |
| 30 | 4.03 | 1.85 | 1.19 | 0.903 | 0.308 |
| 36 | 3.26 | 2.33 | 1.69 | 0.702 | 0.822 |
| 48 | 2.88 | 2.43 | 1.09 | 1.3 | 1.13 |
| 72 | 2.94 | 1.95 | 1.19 | 1.31 | 1.13 |
| 104 | 2.8 | 2.73 | 0.993 | 0.803 | 0.719 |

TABLE 10
Regimen #2: Percent of Patients Attaining LVOT ≤
30 mmHg Across Time Points During the 104-Week Dosing
Period Stratified by Phenotype

| Week | PM | IM | NM | RM | UM |
|---|---|---|---|---|---|
| 4 | 75.6 | 55.8 | 48.2 | 49.2 | 38 |
| 8 | 84.7 | 62.8 | 48.6 | 51.2 | 39 |
| 10 | 82.2 | 59.6 | 44.3 | 45.3 | 34.7 |
| 12 | 73.8 | 50 | 40.4 | 41.2 | 32.7 |
| 18 | 83.9 | 71.7 | 63.5 | 61.1 | 55.8 |
| 24 | 88.2 | 72.9 | 62.7 | 61 | 58.5 |
| 30 | 88.4 | 81.1 | 75.3 | 75.2 | 69.8 |
| 36 | 89.2 | 80.4 | 78.3 | 75.4 | 71.3 |
| 48 | 91 | 87.3 | 80.5 | 82.1 | 77.6 |
| 72 | 91.5 | 90.4 | 81.8 | 85.8 | 82.3 |
| 104 | 93.6 | 92.8 | 86.1 | 88.3 | 84.9 |

TABLE 11
Regimen #2: Percent of Patients Attaining Mavacamten
Plasma Concentration > 1000 ng/mL Across Time Points
During the 104-Week Dosing Period Stratified by Phenotype

| Week | PM | IM | NM | RM | UM |
|---|---|---|---|---|---|
| 4 | 0.101 | 0 | 0 | 0 | 0 |
| 8 | 1.11 | 0 | 0 | 0 | 0 |
| 10 | 0.302 | 0 | 0 | 0 | 0 |
| 12 | 0.302 | 0 | 0 | 0 | 0 |
| 18 | 1.01 | 0.0972 | 0 | 0.1 | 0 |
| 24 | 2.05 | 0.583 | 0.199 | 0.401 | 0 |
| 30 | 1.65 | 0.292 | 0.497 | 0.1 | 0.103 |
| 36 | 2 | 0.68 | 0.596 | 0.201 | 0.103 |
| 48 | 2.24 | 0.875 | 0.298 | 0.301 | 0 |
| 72 | 2.4 | 0.974 | 0.993 | 0.502 | 0.103 |
| 104 | 2.13 | 1.36 | 1.09 | 0.502 | 0.103 |

FIG. 14 (A-D) shows the Time-Course of Percent of Patients with (A) LVEF<=50%, (B) VLVOT<=30 mmHg, (C) Mavacamten Plasma Concentration >700 ng/mL, and (D) Mavacamten Plasma Concentration >1000 ng/mL (104-Week Treatment Duration) on Regimen #2.

TABLE 12
Regimen #2: Dose Distribution
at Week 104 Stratified by Phenotype

| CYP | DC | 0 mg | 2.5 mg | 5 mg | 10 mg | 15 mg |
|---|---|---|---|---|---|---|
| PM | 10.16 | 0.00 | 50.20 | 34.10 | 7.04 | 0.50 |
| IM | 0.29 | 0.00 | 15.26 | 37.90 | 33.62 | 13.02 |
| NM | 0.00 | 0.00 | 7.25 | 25.62 | 38.23 | 28.90 |
| RM | 0.10 | 0.00 | 7.62 | 27.68 | 35.51 | 29.09 |
| UM | 0.00 | 0.00 | 3.70 | 15.72 | 34.12 | 46.45 |

Regimen #3: Evaluation of Lower Starting Dose with Additional ECHO Monitoring

This regimen remains identical to the regimen defined as “Regimen #2” above, with a lower starting dose of 2.5 mg. This regimen was evaluated in order to assess whether simple recommendations, such as lower starting dose could be made for patients who were known CYP2C19 PMs. For the purposes of this simulation, all patients were started on a 2.5 mg starting dose, however the intent was to assess whether a sub-group of phenotypes may benefit from a lower starting dose with no other modifications to the titration regimen.

Table 13 shows the percentage of patients attaining an LVEF≤50% across selected time points of the 104-week period stratified by phenotype. Generally, the highest percentage of patients with LVEF≤50% was based on phenotype status, with the greatest occurrence in PMs (4.9% at Week 24) and the lowest occurrence in UMs (≤1% at all time points). This relationship was also seen with LVOT, with the highest percentage of patients meeting LVOT≤30 mmHg in PMs and the lowest percentage in UMs (Table 14). In all phenotypes except for UM, >60% of patients met LVOT≤30 mmHg after Week 30.

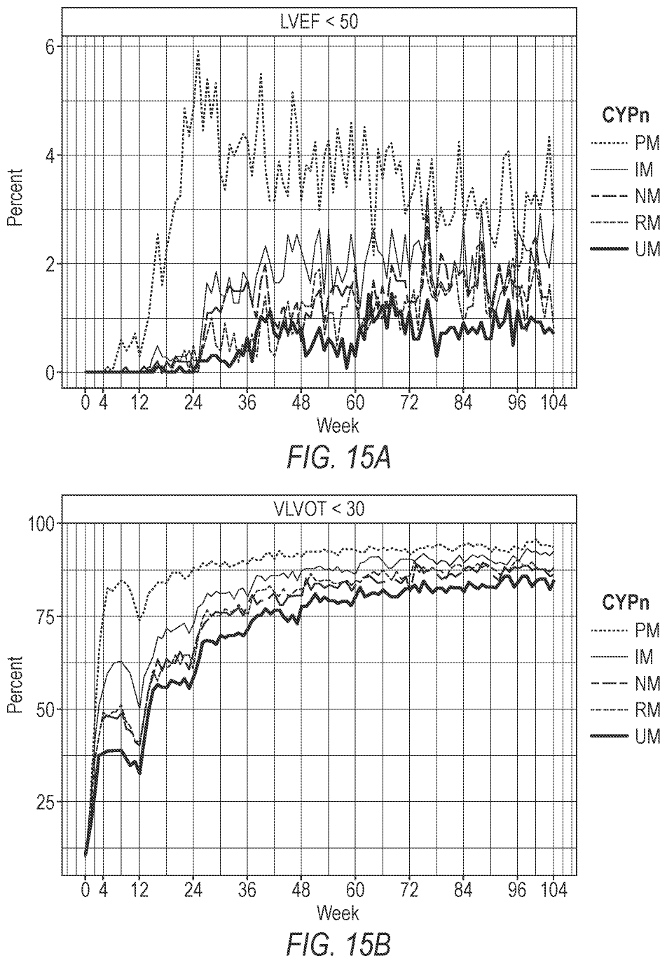

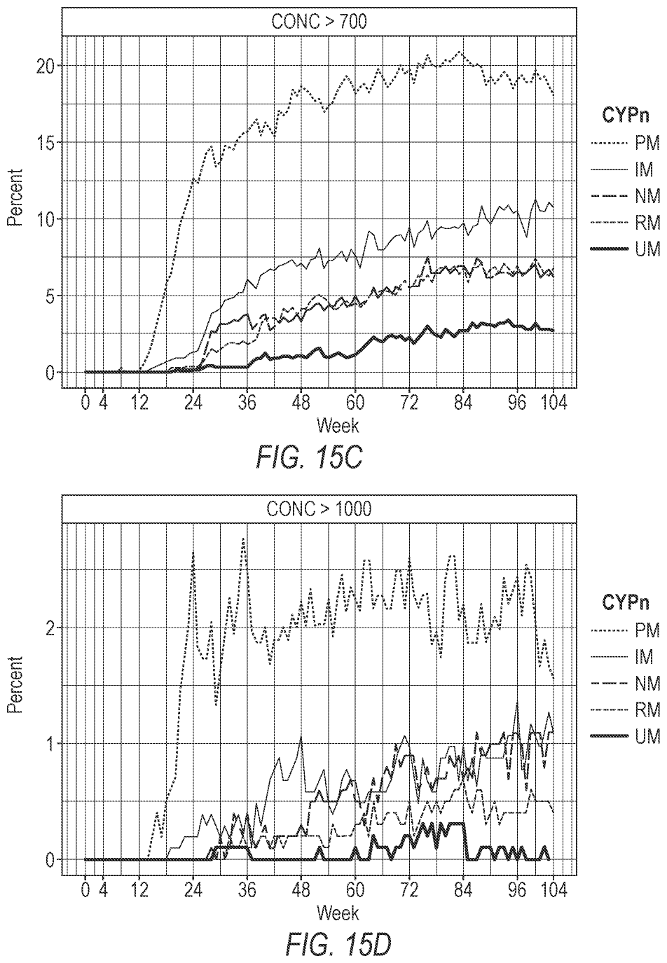

Table 15 shows that the percentage of patients attaining mavacamten plasma concentration >1000 ng/mL was generally low, but higher than Regimen 2 above with no more than 2.6% exceeding this threshold across the phenotypes. FIG. 15 depicts the key safety and efficacy thresholds including mavacamten plasma concentration >700 ng/mL over the 104-week period. The dose distribution depicted in Table 16 is very consistent with the distribution obtained with Regimen 2 above, indicating that over time the same patients eventually achieve similar individualized optimal doses. This regimen comes with a delay in efficacy for patients with no improvement in safety compared to Regimen 2 above. The primary reason for this, is that, in Regimen 2, the introduction of the additional down titration visit at Week 8 allows for an increased opportunity to down titrate patients early, who may eventually require lower doses. Starting patients, especially PMs at a lower dose, eliminates the benefit of the additional down titration opportunity, and when they do eventually get up-titrated, at Week 12 or beyond, the duration of treatment without ECHO visits is prolonged compared to the first 12 weeks, which results in a higher percent of PMs achieving LVEF≤50% overall, but later in therapy i.e. at Week 24 rather than Week 8. Table 16 shows that there is a wide spread of doses across all phenotypes assigned in the simulated patients at Week 104, indicating no single dose strength is appropriate for all patients using the proposed posology.

TABLE 13
Regimen #3: Percent of Patients Attaining
LVEF ≤ 50% Across Time Points During the 104-
Week Dosing Period Stratified by Phenotype

| Week | PM | IM | NM | RM | UM |
|---|---|---|---|---|---|
| 4 | 0 | 0 | 0 | 0 | 0 |
| 8 | 0.604 | 0 | 0 | 0 | 0 |
| 10 | 0.503 | 0 | 0 | 0 | 0 |
| 12 | 0.302 | 0 | 0 | 0 | 0 |
| 18 | 2.23 | 0.292 | 0.199 | 0 | 0 |
| 24 | 4.88 | 0.292 | 0 | 0.502 | 0 |
| 30 | 3.68 | 1.46 | 1.09 | 0.401 | 0.206 |
| 36 | 4.25 | 1.65 | 1.69 | 0.401 | 0.617 |
| 48 | 3.18 | 2.24 | 0.596 | 1.3 | 0.822 |
| 72 | 3.15 | 1.85 | 1.19 | 1.2 | 1.13 |
| 104 | 2.89 | 2.73 | 0.894 | 0.803 | 0.719 |

TABLE 14
Regimen #3: Percent of Patients Attaining LVOT ≤
30 mmHg Across Time Points During the 104-Week Dosing
Period Stratified by Phenotype

| Week | PM | IM | NM | RM | UM |
|---|---|---|---|---|---|
| 4 | 43.5 | 29 | 27 | 26 | 21.3 |
| 8 | 54.2 | 34.8 | 26 | 29.1 | 22.5 |
| 10 | 60.8 | 36.2 | 28.4 | 28.4 | 22 |
| 12 | 60.3 | 34.8 | 27 | 27.7 | 21.6 |
| 18 | 79.4 | 60 | 48.3 | 47.6 | 37.3 |
| 24 | 85.2 | 61.3 | 47.1 | 48 | 41 |
| 30 | 88.7 | 75.9 | 67.7 | 67.9 | 58.6 |
| 36 | 89 | 75.4 | 70.8 | 68 | 59.4 |
| 48 | 91.3 | 84.7 | 75.9 | 77.6 | 71.6 |
| 72 | 91.6 | 90 | 80.6 | 83.7 | 81 |
| 104 | 93.8 | 92.2 | 85.6 | 87.9 | 84.5 |

TABLE 15
Regimen #3: Percent of Patients Attaining Mavacamten
Plasma Concentration > 1000 ng/mL Across Time Points
During the 104-Week Dosing Period Stratified by Phenotype

| Week | PM | IM | NM | RM | UM |
|---|---|---|---|---|---|
| 4 | 0 | 0 | 0 | 0 | 0 |
| 8 | 0 | 0 | 0 | 0 | 0 |
| 10 | 0 | 0 | 0 | 0 | 0 |
| 12 | 0 | 0 | 0 | 0 | 0 |
| 18 | 0.506 | 0 | 0 | 0 | 0 |
| 24 | 2.64 | 0.194 | 0 | 0 | 0 |
| 30 | 1.64 | 0.194 | 0.199 | 0 | 0.103 |
| 36 | 2.49 | 0.389 | 0.397 | 0.1 | 0.103 |
| 48 | 2.22 | 1.07 | 0.298 | 0.201 | 0 |
| 72 | 2.6 | 0.974 | 0.894 | 0.502 | 0.206 |
| 104 | 1.56 | 1.07 | 1.09 | 0.402 | 0 |

FIG. 15 (A-D) shows the Time-Course of Percent of Patients with (A) LVEF<=50%, (B) VLVOT<=30 mmHg, (C) Mavacamten Plasma Concentration >700 ng/mL, and (D) Mavacamten Plasma Concentration >1000 ng/mL (104-Week Treatment Duration) for Regimen #3.

TABLE 16
Regimen #3: Dose Distribution
at Week 104 Stratified by Phenotype

| CYP | DC | 0 mg | 2.5 mg | 5 mg | 10 mg | 15 mg |
|---|---|---|---|---|---|---|
| PM | 9.56 | 0.00 | 50.10 | 35.21 | 6.74 | 0.50 |
| IM | 0.29 | 0.00 | 14.87 | 39.75 | 33.53 | 11.66 |
| NM | 0.00 | 0.00 | 7.25 | 26.71 | 38.43 | 27.61 |
| RM | 0.10 | 0.00 | 7.02 | 29.09 | 36.61 | 27.18 |
| UM | 0.00 | 0.00 | 4.01 | 15.93 | 35.35 | 44.71 |

### Summary

In summary, several titration regimens were assessed to improve the proposed titration regimen. Regimens evaluated assessed monitoring frequency, phenotype specific fixed dose, phenotype specific starting dose, phenotype specific titration rules as well as permanent discontinuation criteria. The second proposed posology (Regimen #2) is considered to provide the best benefit/risk across the population, while improving the safety profile particularly for PMs. Regimen #3 was tested to assess whether simple modifications/recommendations could be made for those patients whose CYP2C19 phenotype was known. A lower starting dose recommendation for PMs did not result in an improvement in benefit/risk. It resulted in a delay in achievement of VLVOT≤30 mmHg as well as a higher % of patients at one visit with LVEF≤50%. While the safety profile appeared improved within the first 12 weeks, the overall percent of patients achieving LVEF≤50% was higher than Regimen #2. However, this phenomenon occurred later, as expected, after patients were up-titrated at week 12. This highlights that a simple modification to the titration scheme such as a lower starting dose does not improve the safety profile for PMs. Additional changes to dosing regimen based on phenotype may result in a slight benefit for PMs but would introduce additional risks for the overall patient population in terms of label complexity, risk of medication errors, and its impact on safety. Balancing all those components, it was assessed that this unified posology in Regimen #2 allowed for an improvement in benefit/risk without a label that introduces too much complexity that could result in unintended medication errors.

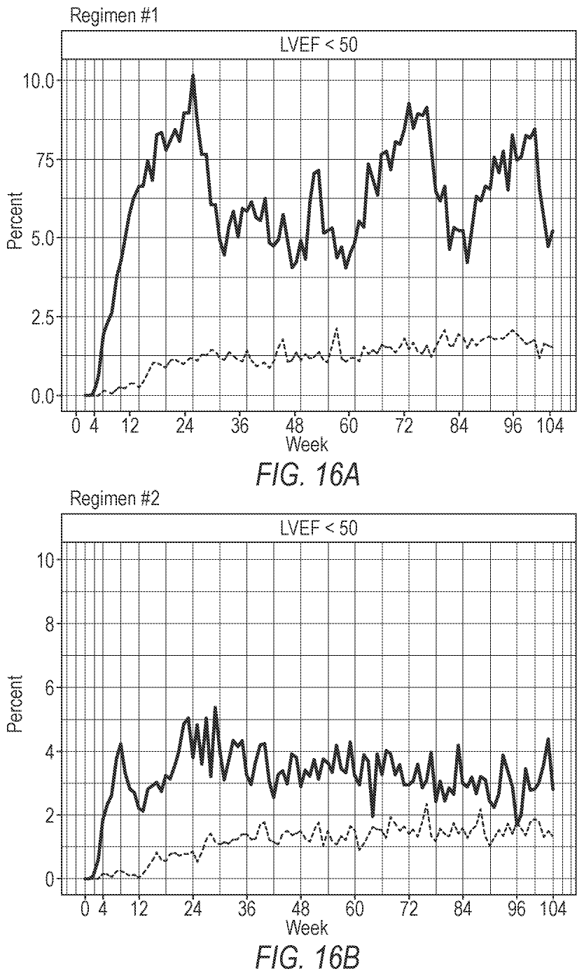

FIG. 16 (A and B) shows a comparison of the Regimen #1 versus the Regimen #2 with respect to the percent of patients for whom LVEF reached below 50%. As seen in the figure, a lower percent of patients, especially poor metabolizer patients, have LVEF<50% under Regimen #2. For example, under Regimen #1, about 10% of poor metabolizer (PM) patients and about 1.2% of non-PM patients have LVEF<50% at Week 24, whereas under Regimen #2, about 3.8% of PM patients and about 0.85% of non-PM patients have LVEF<50% at Week 24. Also, under Regimen #1, about 7% of poor metabolizer (PM) patients and about 0.25% of non-PM patients have LVEF<50% at Week 12, whereas under Regimen #2, about 2.2% of PM patients and about 0.025% of non-PM patients have LVEF<50% at Week 12.

### Example 3. Risk Evaluation and Mitigation Strategy (REMS)

I. REMS Goal

The goal of the MAVACAMTEN Risk Evaluation and Mitigation Strategy (REMS) program is to mitigate the risk of heart failure due to systolic dysfunction.

Objectives:
1. Monitor for detection of heart failure due to systolic dysfunction with periodic echocardiograms.
2. Screen for drug interactions prior to each dispense.

II. REMS Requirements

REMS Program Manager must ensure that healthcare providers, patients, pharmacies, and wholesalers-distributors comply with the following requirements:

1. Healthcare providers who prescribe MAVACAMTEN must:

| | | |
|---|---|---|
| To become | 1. | Review the drug's Prescribing Information. |
| certified | 2. | Review the Program Overview and the |
| to prescribe | | Education Program for Healthcare Providers |
| | | and Pharmacies. |
| | 3. | Successfully complete the Healthcare |
| | | Provider Knowledge Assessment and submit |
| | | it to the REMS. |
| | 4. | Enroll by completing the Healthcare |
| | | Provider Enrollment Form and submitting |
| | | it to the REMS. |
| Before treatment | 5. | Counsel the patient using the Patient |
| initiation | | Brochure on the risk of heart failure |
| (first dose) | | due to systolic dysfunction. |
| | 6. | Counsel the patient using the Patient |
| | | Brochure on the risk of drug-drug |
| | | interactions with CYP2C19 and CYP3A4 |
| | | inhibitors and inducers and the need |
| | | to inform healthcare providers of all |
| | | the prescription and nonprescription |
| | | medications they take. |
| | 7. | Provide the patient with the Patient |
| | | Brochure. |
| | 8. | Assess the patient's cardiovascular status |
| | | and the appropriateness of initiating |
| | | treatment by obtaining an echocardiogram. |
| | | Document and submit confirmation of an |
| | | echocardiogram and authorization for |
| | | treatment to the REMS using the Patient |
| | | Enrollment Form. |
| | 9. | Assess the patient's prescription and |
| | | nonprescription medications and |
| | | supplements for drug-drug interactions. |
| | | Document and submit to the REMS using |
| | | the Patient Enrollment Form. |
| | 10. | Enroll the patient by completing and |
| | | submitting the Patient Enrollment Form |
| | | to the REMS. |
| During treatment: | 11. | Counsel the patient on the risks of |
| 4, 8, and 12 weeks | | heart failure due to systolic dysfunction |
| after treatment | | and drug-drug interactions with CYP2C19 |
| initiation and | | and CYP3A4 inhibitors and inducers, and |
| every 12 weeks | | the related safe-use requirements using |
| thereafter unless | | the Patient Brochure. |
| dose change, | 12. | Assess the patient's cardiovascular |
| initiating weak | | status and the appropriateness of |
| CYP2C19 inhibitor, | | continuing treatment by echocardiogram. |
| or initiating a | | Document and submit confirmation of an |
| moderate CYP3A4 | | echocardiogram and authorization for |
| inhibitor | | treatment to the REMS using the Patient |
| | | Status Form. |
| | 13. | Assess the patient's prescription and |
| | | nonprescription medications and |
| | | supplements for drug-drug interactions. |
| | | Document and submit authorization for |
| | | continuing treatment to the REMS using |
| | | the Patient Status Form. |
| During treatment: | 14. | Counsel the patient on the risks of |
| 4 and 12 weeks | | heart failure due to systolic dysfunction |
| after any dose | | and drug-drug interactions with CYP2C19 |
| change, initiating | | and CYP3A4 inhibitors and inducers, and |
| a weak CYP2C19 | | the related safe-use requirements using |
| inhibitor, or | | the Patient Brochure. |
| initiating a | 15. | Assess the patient's cardiovascular |
| moderate CYP3A4 | | status and the appropriateness of |
| inhibitor, and | | continuing treatment by echocardiogram. |
| every 12 weeks | | Document and submit confirmation of an |
| thereafter | | echocardiogram and authorization for |
| | | continuing treatment to the REMS using |
| | | the Patient Status Form. |
| | 16. | Assess the patient's prescription and |
| | | nonprescription medications and |
| | | supplements for drug-drug interactions. |
| | | Document and submit authorization for |
| | | continuing treatment to the REMS using |
| | | the Patient Status Form. |
| At all times | 17. | Report adverse events of heart failure |
| | | due to systolic dysfunction |

2. Patients who are prescribed MAVACAMTEN:

| | | |
|---|---|---|
| Before treatment | 1. | Receive counseling from the healthcare |
| initiation (first | | provider on the risk of heart failure due |
| dose) | | to systolic dysfunction (when the heart |
| | | is unable to pump enough blood to the |
| | | body) using the Patient Brochure. |
| | 2. | Receive counseling from the healthcare |
| | | provider on the risk of drug-drug |
| | | interactions and the need to inform |
| | | healthcare providers of all prescription |
| | | and over-the-counter medicines and |
| | | supplements they take, using the |
| | | Patient Brochure. |
| | 3. | Get an echocardiogram to check their |
| | | heart. |
| | 4. | Enroll in the REMS by completing the |
| | | Patient Enrollment Form with the |
| | | healthcare provider. Enrollment |
| | | information will be provided to the REMS. |
| During treatment: | 5. | Receive counseling from the healthcare |
| 4, 8, and 12 weeks | | provider on the risk of heart failure |
| after treatment | | due to systolic dysfunction (when the |
| initiation and | | heart is unable to pump enough blood to |
| every 12 weeks | | the body). |
| thereafter unless | 6. | Receive counseling from the healthcare |
| dose change or | | provider on the risk of drug-drug |
| starting certain | | interactions and the need to inform |
| new medicines | | healthcare providers of all the |
| | | prescription and over-the-counter |
| | | medicines and supplements they take, |
| | | using the Patient Brochure. |
| | 7. | Get an echocardiogram to check their |
| | | heart. |
| During treatment: | 8. | Receive counseling from the healthcare |
| 4 and 12 weeks | | provider on the risk of heart failure |
| after dose | | due to systolic dysfunction. |
| changes and | 9. | Receive counseling from the healthcare |
| starting certain | | provider on the risk of drug-drug |
| new medicines and | | interactions and the need to inform |
| every 12 weeks | | healthcare providers of all the |
| thereafter | | prescription and over-the-counter |
| | | medicines and supplements they take, |
| | | using the Patient Brochure. |
| | 10. | Get an echocardiogram to check their |
| | | heart. |
| Before each | 11. | Review all prescriptions and over-the- |
| prescription | | counter medicines and supplements with |
| dispense | | the pharmacist. |
| | 12. | Receive counseling from the pharmacist |
| | | on drug-drug interactions. |
| At all times | 13. | Inform the healthcare provider or seek |
| | | other medical attention if there are new |
| | | or worsening symptoms of heart failure. |
| | 14. | Inform other healthcare providers about |
| | | treatment with MAVACAMTEN. |
| | 15. | Inform healthcare providers of all |
| | | medicines and any changes, including |
| | | over-the-counter medicines and supplements. |

3. Pharmacies that dispense MAVACAMTEN must:

| | | |
|---|---|---|
| To become | 1. | Designate an authorized representative |
| certified to | | to complete the certification process |
| dispense | | and oversee implementation and compliance |
| | | with the REMS on behalf of the pharmacy. |
| | 2. | Have the authorized representative review |
| | | the Prescribing Information, the Education |
| | | Program for Healthcare Providers and |
| | | Pharmacies, and the Program Overview. |
| | 3. | Have the authorized representative |
| | | successfully complete the Pharmacy |
| | | Authorized Representative Knowledge |
| | | Assessment and submit it to the REMS. |
| | 4. | Have the authorized representative enroll |
| | | in the REMS on behalf of the pharmacy by |
| | | completing the Pharmacy Enrollment Form |
| | | and submitting it to the REMS. |
| | 5. | Train all relevant staff involved in |
| | | dispensing MAVACAMTEN using the Program |
| | | Overview and Education Program for |
| | | Healthcare Providers and Pharmacies. |
| Before dispensing | 6. | Counsel the patient on drug-drug |
| | | interactions. |
| | 7. | Assess the patient's prescription and |
| | | nonprescription medications and |
| | | supplements for drug-drug interactions. |
| | | Document and submit to the REMS using the |
| | | Drug Interaction and Counseling Checklist |
| | | for Pharmacies. |
| | 8. | Document the prescribed dose. |
| | 9. | Obtain authorization to dispense each |
| | | prescription by contacting the REMS to |
| | | verify that the prescriber is certified, |
| | | the patient is enrolled, the healthcare |
| | | provider has authorized the patient to |
| | | receive the drug, the patient is counseled, |
| | | and the pharmacy identified and resolved |
| | | any drug-drug interactions. |
| | 10. | Provide the patient with the Patient |
| | | Brochure. |
| | 11. | Dispense no more than a 35-day supply of |
| | | MAVACAMTEN. |
| To maintain | 12. | Have the new authorized representative |
| certification | | enroll in the REMS by successfully |
| to dispense | | completing the Pharmacy Authorized |
| | | Representative Knowledge Assessment and |
| | | completing the Pharmacy Enrollment |
| | | Form if the authorized representative |
| | | changes. |
| At all times | 13. | Report adverse events of heart failure |
| | | due to systolic dysfunction |
| | 14. | Do not distribute, transfer, loan, or sell |
| | | MAVACAMTEN, except to a certified pharmacy. |
| | 15. | Maintain records of dispensing information. |
| | 16. | Maintain records of completion of the REMS |
| | | training by relevant staff. |
| | 17. | Maintain records that all processes and |
| | | procedures are in place and are being |
| | | followed. |
| | 18. | Comply with audits conducted by REMS |
| | | Program Manager or a third party acting |
| | | on behalf of REMS Program Manager to ensure |
| | | that all processes and procedures are in |
| | | place and are being followed. |

4. Wholesalers-distributors that distribute MAVACAMTEN must:

| | | |
|---|---|---|
| To be able to | 1. | Establish processes and procedures to ensure that |
| distribute | | the drug is distributed only to certified pharmacies. |
| | 2. | Train all relevant staff involved in distributing |
| | | MAVACAMTEN on the REMS requirements. |
| At all times | 3. | Distribute only to certified pharmacies. |
| | 4. | Maintain records of drug distribution for all |
| | | MAVACAMTEN shipments. |
| | 5. | Comply with audits conducted by REMS Program |
| | | Manager or a third party acting on behalf of REMS |
| | | Program Manager to ensure that all processes and |
| | | procedures are in place and are being followed. |

REMS Program Manager must provide training to healthcare providers who prescribe MAVACAMTEN.

The training includes the following educational materials:
Program Overview
Education Program for Healthcare Providers and Pharmacies
Healthcare Provider Knowledge Assessment

The training must be available online, via email, and in a hard-copy format via mail.

REMS Program Manager must provide training to pharmacies that dispense MAVACAMTEN.

The training includes the following educational materials:
Program Overview
Education Program for Healthcare Providers and Pharmacies
Pharmacy Authorized Representative Knowledge Assessment

The training must be available online, via email, and in a hard-copy format via mail.

To support REMS operations, REMS Program Manager must:
1. Authorize dispensing for each patient based on receipt of a Patient Enrollment Form, Patient Status Form, and Drug Interaction and Counseling Checklist for Pharmacies on the following schedule:
   For the initial dispense: If the Patient Enrollment Form and Drug Interaction and Counseling Checklist for Pharmacies are not completed, the patient is not authorized to receive drug until the completed forms are received.
   For subsequent dispenses: If the Patient Status Form is not received within 3 calendar days of the last day of the week the echocardiogram is due (as described in the Education Program for Healthcare Providers and Pharmacies) and the Drug Interaction and Counseling Checklist for Pharmacies is not completed before each dispense, the patient is not authorized to receive the drug until the completed forms are received.
2. Establish and maintain a REMS Website. The REMS Website must include the capability to complete healthcare provider and pharmacy certification and enrollment online; the capability to enroll and manage patients online, including completion of the Drug Interaction and Counseling Checklist for Pharmacies and Patient Status Form; the capability to review patient enrollment and authorization status and healthcare provider certification status, as well as obtaining authorization to dispense; and the option to print the Prescribing Information, Medication Guide, and REMS materials. All product websites for consumers and healthcare providers must include prominent REMS-specific links to the REMS Program website. The REMS Program website must not link back to the promotional product website(s).
3. Make the REMS Website fully operational and all REMS materials available through website and the MAVACAMTEN REMS Call Center at the time that MAVACAMTEN first becomes commercially available.
4. Establish and maintain a REMS Call Center for REMS participants.
5. Establish and maintain a validated, secure database of all REMS participants who are enrolled and/or certified in the MAVACAMTEN REMS.
6. Ensure healthcare providers and pharmacies are able to complete the certification process online and by fax.
7. Ensure healthcare providers are able to complete the patient enrollment process online and by fax.
8. Ensure healthcare providers are able to complete the Patient Status Form online and by fax.
9. Ensure pharmacies are able to document the prescribed dose and complete the Drug Interaction and Counseling Checklist for Pharmacies online and by fax.
10. Ensure pharmacies are able to obtain authorization to dispense online and by phone.
11. Provide the Program Overview, Education Program for Healthcare Providers and Pharmacies, and the Healthcare Provider Enrollment Form or Pharmacy Enrollment Form to healthcare providers who (1) attempt to prescribe or dispense MAVACAMTEN and are not yet certified or (2) inquire about how to become certified.
12. Notify healthcare providers and pharmacies within 1 business day when they become certified in the REMS.
13. Notify healthcare providers within 1 business day when patient enrollment is complete.
14. Provide certified healthcare providers access to the database of their enrolled patients and certified pharmacies.
15. Provide certified pharmacies access to the database of certified healthcare providers and enrolled patients.
16. Provide authorized wholesalers-distributors access to the database of certified pharmacies.

To ensure REMS participants' compliance with the REMS, REMS Program Manager must:
17. Annually verify that the authorized representative's name and contact information correspond to those of the current designated authorized representative for the pharmacy. If different, the pharmacy must recertify a new authorized representative.
18. Maintain adequate records to demonstrate that the REMS requirements have been met, including but not limited to records of the following: MAVACAMTEN distribution and dispensing, certification of healthcare providers and pharmacies, enrolled patients, completed Patient Status Forms and Drug Interaction and Counseling Checklist for Pharmacies, and audits of certified pharmacies and wholesalers-distributors. These records must be readily available for FDA inspections.
19. Establish and maintain a plan for addressing noncompliance with the REMS requirements.
20. Monitor healthcare providers, pharmacies, and wholesalers-distributors on an ongoing basis to ensure the REMS requirements are being met. Take corrective action if noncompliance is identified, including decertification.
21. Audit pharmacies and wholesalers-distributors no later than 180 calendar days after they become certified and annually to ensure all REMS processes and procedures are in place, functioning, and support the REMS requirements.
22. Take reasonable steps to improve operations of and compliance with the REMS requirements based on monitoring and evaluation of the MAVACAMTEN REMS.

III. REMS Assessment Timetable

REMS Program Manager must submit REMS assessments annually from the date of the initial approval of the REMS program. To facilitate the inclusion of as much information as possible while allowing reasonable time to prepare the submission, the reporting interval covered by each assessment should conclude no earlier than 60 calendar days before the submission date for that assessment. REMS Program Manager must submit each assessment so that it will be received by the FDA on or before the due date.

IV. REMS Materials

The following materials are part of the MAVACAMTEN REMS:

### Enrollment Forms

Healthcare Providers:
1. Healthcare Provider Enrollment Form

Patients:
2. Patient Enrollment Form

Pharmacy:
3. Pharmacy Enrollment Form

### Training and Educational Materials

Healthcare Providers:
4. Education Program for Healthcare Providers and Pharmacies
5. Program Overview
6. Healthcare Provider Knowledge Assessment

Patients:
7. Patient Brochure

Pharmacy:
8. Education Program for Healthcare Providers and Pharmacies
9. Program Overview
10. Pharmacy Authorized Representative Knowledge Assessment

### Patient Care Forms

11. Patient Status Form
12. Drug Interaction and Counseling Checklist for Pharmacies

### Other Materials

13. REMS Website

### Example 4. Drug-Drug Interaction Simulations

Statement of Purpose: Mavacamten is a first-in-class, selective, allosteric, and reversible cardiac myosin inhibitor in development for the treatment of adults with symptomatic obstructive hypertrophic cardiomyopathy. Mavacamten is hepatically metabolized predominantly via CYP2C19 (74%), CYP3A4 (18%) and CYP2C9 (8%). A previously developed fit-for-purpose physiologically based pharmacokinetic (PBPK) model was applied to explore the induction effects of a strong CYP3A4/CYP2C19 inducer—rifampin, as well as a strong CYP3A4 inducer—carbamazepine, on mavacamten exposures.

Methods: Modeling and simulation analyses were conducted using Simcyp v19 (Certara). The mavacamten compound file was further verified against additional mavacamten Phase I clinical data in healthy volunteers. The DDI simulation study design included lead-in inducer dosing period to maximize induction effects (rifampin 600 mg QD for 7 days, carbamazepine 400 mg BID for 14 days) followed by a single mavacamten dose (15 mg), with continued dosing of inducers for another 2 months to end of virtual trial. All simulations used the default rifampin and carbamazepine compound files provided in Simcyp Simulator (V19). The results of the simulations were stratified by 3 populations: healthy volunteers containing all CYP2C19 phenotypes, healthy volunteers excluding CYP2C19 poor metabolizers (PM), healthy volunteers including only CYP2C19 PM.

Data & Results: The rifampin DDI simulations observed a moderate reduction of mavacamten AUC_{0-T} (geometric mean (GM) 60-69%), and minimal reduction in Cmax (GM 4-7%) for all virtual populations evaluated. Mavacamten clearance increased by 2.5 to 3.2-fold in the presence of rifampin induction. DDI simulation with carbamazepine resulted in a weak reduction of mavacamten AUC_{0-T} (GM 13-30%) and minimal reduction in Cmax (GM 1%) for all virtual populations evaluated. Mavacamten clearance was increased by 1.1 to 1.4-fold in the presence of carbamazepine induction.

Conclusions: The Simcyp Simulator was used to investigate the impact of strong CYP2C19 and/or CYP3A4 induction on mavacamten exposures based on CYP2C19 phenotypic distribution in a virtual population. Application of the model, to understand impact of strong CYP2C19/CYP3A4 induction (rifampin), revealed moderate reductions in mavacamten exposure in all populations. A weak induction effect in all populations was observed when co-administering mavacamten with carbamazepine, a less potent strong CYP3A4 inducer. Due to the reductions in mavacamten exposure when co-administered with strong CYP2C19 and/or CYP3A4 inducers, mavacamten is proposed to be contraindicated with both strong and moderate inducers of CYP2C19 and CYP3A4.

### Example 5. Concomitant Administration Simulation

Statement of Purpose: Mavacamten is a first-in-class, allosteric, selective, and reversible cardiac myosin inhibitor currently in development for the treatment of adults with symptomatic obstructive hypertrophic cardiomyopathy. A physiologically based pharmacokinetic (PBPK) model was utilized to explore mavacamten victim drug-drug interaction (DDI) status with CYP2C19 and CYP3A4 induction. The PBPK model was further implemented to explore potential effects of CYP2C19 and CYP3A4 inhibitors on mavacamten pharmacokinetics.

Methods: All modeling was performed using Simcyp v19 software (Certara). A previously developed fit-for-purpose PBPK model was verified with additional clinical data from amavacamten DDI study with CYP3A4 inhibitor verapamil. Using Simcyp library compounds of ticlopidine, omeprazole, itraconazole, diltiazem, and cimetidine, strong, moderate, and weak inhibition of CYP2C19 and CYP3A4 were explored individually to understand potential effects on mavacamten exposure with the maximum suggested clinical dose (15 mg QD). Simulations were run under steady-state conditions for both mavacamten and perpetrator and DDI potential explored per CYP2C19 phenotype.

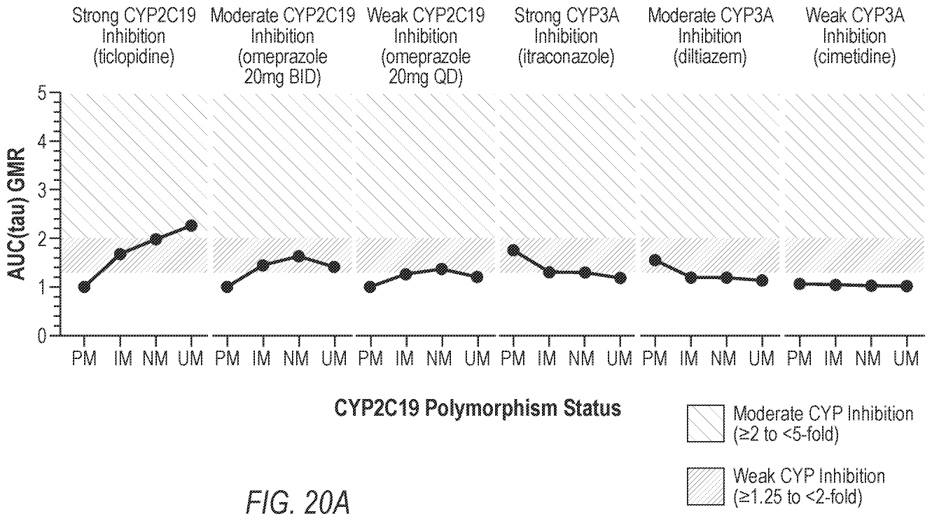

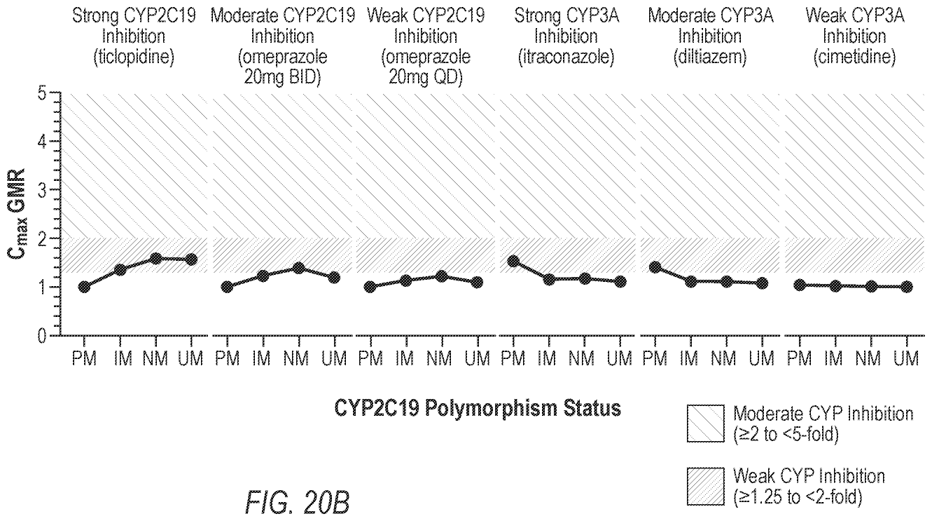

Data & Results: Simulations of strong CYP2C19 or CYP3A4 inhibition resulted in geometric mean AUC increases of up to 2.3- and 1.8-fold, respectively, and geometric mean Cmax increases of up to 1.6- and 1.5-fold, respectively, with inhibition demonstrating phenotype-specific trends, e.g., strong CY-P2C19 inhibition impacted CYP2C19 ultra-rapid metabolizers the most while strong CYP3A4 inhibition had the greatest effect on poor metabolizers (FIG. 20 A & B). Moderate CYP2C19 or CYP3A4 inhibition was predicted to increase AUC up to 1.6-fold and Cmax up to 1.4-fold. Weak CYP2C19 inhibition was predicted to increase AUC and Cmax at most 1.4- and 1.2-fold, respectively, while weak CYP3A4 inhibition was predicted to increase mavacamten exposure less than 1.1-fold. Results are shown in FIG. 20 (A & B).

Conclusions: The simulation results across the CY-P2C19 phenotypes were used to inform the posology of mavacamten in CYP2C19 and CYP3A4 DDI inhibition situations in the patient population since CYP2C19 is a highly polymorphic enzyme. Due to the exposure increases observed in the simulations, contraindication of coadministration of moderate to strong CYP2C19 inhibitors and strong CYP3A4 inhibitors is proposed. Additional proposals for weak CYP2C19 inhibitors or moderate CYP3A4 inhibitors were suggested, including starting mavacamten treatment at the approved recommended dosage in patients on stable inhibitor regimens, and reducing mavacamten dose by one level in patients who intend to start therapy with these inhibitors. In conclusion, PBPK modeling was implemented to inform mavacamten DDI inhibition recommendations considering CYP2C19 phenotype difference.

### Example 6. Risk Evaluation and Mitigation Strategy Requirements

REMS must include the following:

Elements to assure safe use: elements necessary to assure safe use are required as part of the REMS to mitigate the risk of heart failure due to systolic dysfunction listed in the labeling of the drug.

REMS includes the following elements to mitigate this risk:
Healthcare providers have particular experience or training, or are specially certified
Pharmacies, practitioners, or health care settings that dispense the drug are specially certified
The drug is dispensed to patients with evidence or other documentation of safe-use conditions
Each patient using the drug is subject to certain monitoring

Implementation System: The REMS must include an implementation system to monitor, evaluate, and work to improve the implementation of the elements to assure safe use (outlined above) that require: pharmacies that dispense the drug be specially certified, and the drug is dispensed to patients with documentation of safe use conditions.

The REMS consists of elements to assure safe use, an implementation system, and a timetable for submission of assessments of the REMS.

The REMS assessment plan must include, but is not limited to, the following: Program Outreach and Communication (provide data at the 1-year assessment only)

1. REMS Program Website
a) Date REMS website went live
b) Number of total visits and unique visits to the REMS Program Website
c) Number and type of Mavacamten REMS materials downloaded or accessed

Program Implementation and Operations

2. REMS Call Center Reports (Provide Data for Two Previous Reporting Periods, the Current Reporting Period, and Cumulatively)
a) Number of calls by stakeholder type (patient, healthcare provider, designee, pharmacy, wholesalers-distributors, other)
b) Summary of reasons for calls (e.g., enrollment question) and stakeholder type (patients, healthcare provider, designee, pharmacy, other). Limit the summary to the top five reasons for calls by each stakeholder group.
c) If the summary reason for the call(s) indicates a complaint, include details on the nature of the complaint(s) and whether the caller indicated potential REMS burden or patient access issues
d) If the summary reason for the call(s) indicates an adverse event related to heart failure or a contraindicated drug or drug interaction, include details and the outcome of the call(s)
e) Percentage of calls to the REMS Call Center that were answered within 20 minutes
f) The shortest wait time for a call to be answered, the longest wait time for a call to be answered and the median time for a call to be answered
g) Percentage of calls to the REMS Call Center where the caller abandoned the call before the call was answered
f) The shortest wait time at which a call was abandoned, the longest wait time before the call was abandoned and the median wait time for a call to be abandoned

3. Program Implementation (Provide Data at the 1-Year Assessment Only)
a) Date of first commercial availability of Mavacamten
b) For each stakeholder (healthcare providers, designees, pharmacies, patients), the date when they could become certified
c) Date when the Mavacamten REMS Call Center was established and fully operational

4. REMS Certification and Enrollment (Provide Data for Two Previous Reporting Periods, the Current Reporting Period and Cumulatively)
a) Healthcare Providers
i. Number of newly certified healthcare providers and number of active (i.e., who have prescribed at least once during the reporting period) healthcare providers stratified by credentials (e.g., Doctor of Medicine, Doctor of Osteopathic Medicine, Nurse Practitioner, Physician Assistant, Other), specialty (e.g., Cardiology, Electrophysiology, Geneticist, Other), and geographic region (defined by US Census). If “Other” accounts for >10% of respondents, provide the most common specialties identified. Specifically identify and categorize if a specialty is within cardiology or non-cardiology
b) Number of designees stratified by role (e.g., RPh/PharmD, RN, NP, or PA)
i. Method of healthcare provider and designee certification (online or fax)
c) Pharmacies
i. Number of newly certified pharmacies
ii. Number of active pharmacies (i.e., have dispensed Mavacamten)
d) Patients
i. Number of newly enrolled patients and number of active (i.e., received at least one dispense of Mavacamten) patients stratified by a combined variable of age and gender and geographic region. Provide the minimum and maximum age of enrolled patients. For gender/age variable use age ranges of less than 18, 18-40, 41-60, 61 years and older
e) Wholesalers-distributors
i. Number of newly contracted wholesalers-distributors and number of active (i.e., have shipped Mavacamten) wholesalers-distributors

5. REMS Compliance (Provide Data for Two Previous Reporting Periods, the Current Reporting Period, and Cumulatively)
a) A copy of the non-compliance plan, including the criteria for non-compliance for healthcare providers, pharmacies, and wholesalers-distributors, actions taken to address noncompliance for each case, and which events lead to decertification from the Mavacamten REMS (Beginning with the 1-year assessment and annually thereafter)
b) Audits
i. A copy of the audit plan for pharmacies and wholesalers/distributors
ii. Report of audit findings for each stakeholder (pharmacies and wholesalers-distributors)
iii. Number of audits expected, and the number of audits performed.
iv. Documentation of completion of training for relevant staff
v. Documentation of processes and procedures in place for complying with the Mavacamten REMS
vi. Verification for each audited stakeholder's site that the designated Authorized Representative remains the same. If different, document that the pharmacy has re-certified with the name and contact information for the new Authorized Representative
vii. Number and types of deficiencies noted for each group of audited stakeholders as a percentage of audited stakeholders.
viii. For each Audited Pharmacy, number of the following deficiencies (numerator) divided by the number of dispenses audited at that pharmacy (denominator):
   Healthcare provider not certified, and prescription dispensed
   Patient not enrolled and prescription dispensed
   Drug Interaction and Counseling Checklist not completed, and prescription dispensed
   Audit of Drug Interaction and Counseling Checklist forms that identified a drug was dispensed but a required action not taken
   Authorization denied and prescription dispensed
ix. For stakeholders with deficiencies noted, the number that successfully completed a Corrective and Preventative Action (CAPA) plan and as a percentage of those for which a CAPA plan was requested
x. For any stakeholders who did not complete the CAPA Plan, a description of actions taken
c) Healthcare provider noncompliance (For each non-compliance event, the source of the report, a description of the event, the root cause analysis of the event, and corrective actions taken)
i. Number of healthcare providers who were non-compliant with the Mavacamten REMS program requirements. Provide as a percentage of active healthcare providers.
ii. Number of healthcare providers who were de-certified and reasons for de-certification, also provided as a percentage of active healthcare providers. Include if any healthcare providers were re-certified.
d) Pharmacies (For each non-compliance event, the source of the report, a description of the event, the root cause analysis, and corrective actions taken)
i. Number of pharmacies for which non-compliance with the Mavacamten REMS is detected (numerator) divided by all pharmacies dispensing Mavacamten (denominator)
ii. The number of non-certified pharmacies that dispensed Mavacamten (numerator) divided by all pharmacies that dispensed Mavacamten (denominator). A compliance rate of 99.9% is expected.
iii. Number of Mavacamten prescriptions dispensed by non-certified pharmacies (numerator) divided by all Mavacamten prescriptions dispensed (denominator) and the actions taken to prevent future occurrences. A compliance rate of 99.9% is expected
iv. Number of Mavacamten prescriptions dispensed that were written by non-certified healthcare providers (numerator) divided by all dispensed prescriptions (denominator). For prescriptions dispensed that were written by non-certified healthcare providers, provide the root cause analysis and the actions taken to prevent future occurrences. A compliance rate of 99.9% is expected.
v. Number of Mavacamten prescriptions dispensed to non-enrolled patients (numerator) divided by all dispensed prescriptions (denominator). For prescriptions dispensed to non-enrolled patients provide a root cause analysis and the actions taken to prevent future occurrences. A compliance rate of 99.9% is expected.
vi. Number of Mavacamten prescriptions dispensed to non-enrolled patients based on a prescription from a non-certified healthcare provider (numerator) divided by all dispensed prescriptions (denominator). For prescriptions dispensed to non-enrolled patients based on a prescription from a non-certified healthcare provider provide a root cause analysis and the actions taken to prevent future occurrences. A compliance rate of 99.9% is expected.
vi. Number of times a Mavacamten prescription was dispensed because a certified pharmacy bypassed the Mavacamten REMS authorization processes (numerator) divided by all certified pharmacies (denominator). Provide a root cause analysis and include a description of how the events were identified and any corrective actions taken. A compliance rate of 99.9% is expected.
vii. Number of pharmacies decertified, reasons for decertification, and actions to address non-compliance. Provide as a ratio the number of pharmacies decertified (numerator) divided by all certified pharmacies (denominator).
e) Wholesalers-distributors (For each non-compliance event, the source of the report, a description of the event, the root cause analysis, and corrective actions taken)
i. Number of contracted wholesalers-distributors for which non-compliance with the Mavacamten REMS is detected (numerator) divided by the number of contracted wholesalers-distributors (denominator)
ii. Number of wholesalers-distributors suspended from distributing, reasons for the suspension, and actions to address non-compliance
iii. Number of times Mavacamten was distributed to a non-certified pharmacy (numerator) divided by the number of distributions of Mavacamten (denominator)

6. Utilization Data (Provide Data for Two Previous Reporting Periods, the Current Reporting Period, and Cumulatively)
a) Number of prescriptions (new and refills) dispensed, stratified by:
i. Healthcare provider degree/credentials and geographic region
ii. Patient demographics (age and gender, and geographic region)
b) The number of prescriptions received and denied (not authorized), stratified by:
i. Reasons and number of denials (numerator) divided by all denials (denominator)
   Healthcare provider not certified
   Prescription written by designee
   Patient not enrolled
   Patient status form documenting echocardiogram not submitted on appropriate schedule
   Drug Interaction and Counseling Checklist not completed
   Drug interaction or contraindicated drug identified, and appropriate actions not taken
   Other reasons for denial not categorized above
ii. Healthcare provider degree/credentials and geographic region
c) Number of unique healthcare providers who wrote prescriptions dispensed in the reporting period (active healthcare providers)
d) Number of unique patients receiving Mavacamten, stratified by age, gender, and geographic region

7. Burden to the Healthcare System and/or Barriers to Patient Access

Reports to the Mavacamten REMS Call Center indicating a burden to the healthcare system or barriers to patient access. Assessment of whether burden is attributable to the REMS, insurance, health care availability, other

Safe Use Behavior

8. Patient Status Forms (Provide Data for Two Previous Reporting Periods, Current Reporting Period and Cumulatively)
a) Number of Patient Status Forms expected, received, and outstanding as of the REMS assessment cut-off date
b) Number of first patient shipments sent prior to receipt of a Patient Enrollment Form (numerator) divided by all patients who were dispensed Mavacamten (denominator). A compliance rate of 99.9% is expected.
c) Number of unique patients who had a Patient Status Form submitted who the healthcare provider confirmed reviewing the echocardiogram for (numerator) divided by number of unique patients who had a Patient Status Form submitted (denominator)
d) Number of unique patients who had a Patient Status Form submitted who the healthcare provider authorized treatment for (numerator) divided by number of unique patients who had a patient status form submitted (denominator)
e) Number of Patient Status Forms outstanding from previous reporting periods that were completed in the current reporting period (numerator) divided by the number of outstanding Patients Status Forms from the previous reporting period (if applicable)
f) Number of patients whose echocardiogram was completed off drug as a result of an authorization denial and reason (e.g., drug not dispensed due to missing Patient Status Form, insurance issues prevented drug dispensing, transportation issues prevented patient from obtaining echocardiograms)
g) Number of Patient Status Forms on which the healthcare provider indicated that the patient experienced a clinical heart failure event requiring hospitalization
h) Number of Patient Status Forms on which the healthcare provider indicated the patient experienced a decrease in LVEF to <50%
i) Number of patients who were not authorized to receive Mavacamten as indicated on the Patient Status Form

9. Drug Interaction and Counseling Checklist for Pharmacies (Provide Data for Two Previous Reporting Periods, Current Reporting Period and Cumulatively)
a) Number of unique patients who had a Drug Interaction and Counseling Checklist completed prior to their initial dispensing of Mavacamten (numerator) divided by the number of patients who initiated therapy with Mavacamten (denominator). A compliance rate of 99.9% is expected.
b) Number of prescriptions dispensed that had a Drug Interaction and Counseling Checklist completed prior to dispensing (numerator) divided by the number of prescriptions dispensed for Mavacamten (denominator). A compliance rate of 99.9% is expected.
c) Number of Drug Interaction and Counseling Checklists that identified a concurrent contraindicated medicine (numerator) divided by the total number of Drug Interaction and Counseling Checklists completed (denominator)
d) For those Drug Interaction and Counseling Checklists that identified a concurrent contraindicated medicine indicate the source of the drug interaction and action taken after healthcare provider was contacted including:
i. Source
   1. Interacting drug prescribed by Mavacamten certified healthcare provider/designee
   2. Interacting drug prescribed by other healthcare provider
   3. Interacting drug purchased over the counter by patient
ii. Action taken
   1. Mavacamten discontinued
   2. Contraindicated drug discontinued
e) Number of Drug Interaction and Counseling Checklists that identified a concurrent medicine that required a dosage reduction (numerator) divided by the total number of Drug Interaction and Counseling Checklists completed (denominator)
f) For those Drug Interaction and Counseling Checklists that identified a concurrent medicine that required a dosage reduction, indicate the source of the drug interaction and action taken after healthcare provider was contacted including:
i. Source
   1. Interacting drug prescribed by Mavacamten certified healthcare provider/designee
   2. Interacting drug prescribed by other healthcare provider
   3. Interacting drug purchased over the counter by patient
ii. Action taken
   1. Mavacamten discontinued
   2. Mavacamten dose decreased
   3. Other medicine(s) discontinued
g) Any information obtained from audits, or self-reported by pharmacies that indicated that a patient received a contraindicated medicine, while taking Mavacamten expressed by the number of patients who received at least one shipment (dispensing) of Mavacamten who were also taking a concurrent contraindicated medicine (numerator) divided by the total number of patients with at least one shipment (dispensing) of Mavacamten (denominator). For all occurrences, include the contraindicated drug name, dose, and duration of therapy.

10. Knowledge Assessments (Provide Data at the 1-Year and 2-Year Assessment Reports Only)
a) Number of completed Healthcare Provider Knowledge Assessments, including the method of completion and number of attempts to complete
b) A summary of the most frequently missed Healthcare Provider Knowledge Assessment questions
c) A summary of potential comprehension or perception issues identified with the Healthcare Provider Knowledge Assessment
d) Number of completed Pharmacy Authorized Representative Knowledge Assessments, including the method of completion and number of attempts to complete
e) A summary of the most frequently missed Pharmacy Authorized Representative Knowledge Assessment questions
f) A summary of potential comprehension or perception issues identified with the Pharmacy Authorized Representative Knowledge Assessment.

We claim:

1. A method of treating a patient in need thereof with a myosin inhibitor, comprising:
administering a starting dose of the myosin inhibitor to the patient for a first treatment period;
when a first measurement of left ventricular outflow tract obstruction of the patient taken at or near the conclusion of the first treatment period is below a threshold value, administering a first reduced dose of the myosin inhibitor to the patient during a second treatment period wherein the first reduced dose is less than the starting dose; and
when a second measurement of left ventricular outflow tract obstruction of the patient taken at or near the conclusion of the second treatment period is below a threshold value, administering a second reduced dose of the myosin inhibitor to the patient during a third treatment period, wherein the second reduced dose is less than a dose of the myosin inhibitor administered immediately prior to the second reduced dose.

2. The method of claim 1, further comprising
obtaining a first measurement of left ventricular outflow tract obstruction of the patient taken at or near the conclusion of the first treatment period; and
obtaining a second measurement of left ventricular outflow tract obstruction of the patient taken at or near the conclusion of the second treatment period.

3. The method of claim 1 or 2, wherein the second treatment period immediately follows the first treatment period.

4. The method of any one of claims 1-3, wherein the third treatment period immediately follows the second treatment period.

5. The method of any one of claims 1-4, wherein the dose of the myosin inhibitor administered to the patient is not increased until after the third treatment period.

6. The method of any one of claims 1-5, wherein the first and second measurements are taken using echocardiography.

7. The method of any one of claims 1-6, wherein the first measurement of left ventricular outflow tract obstruction is a measurement of Valsalva LVOT gradient.

8. The method of claim 7, wherein the second measurement of left ventricular outflow tract obstruction is a measurement of Valsalva LVOT gradient.

9. The method of claim 7 or 8, wherein the threshold value is a Valsalva LVOT gradient of 20 mmHg.

10. The method of any one of claims 1-9, wherein the patient's risk of an adverse event is reduced as compared to continued administration of the myosin inhibitor at the starting dose.

11. The method of any one of claims 1-10, wherein the first treatment period is about 4 weeks.

12. The method of claim 11, wherein the second treatment period is about 4 weeks.

13. The method of claim 12, wherein the first, second, and third treatment periods are each about 4 weeks.

14. The method of one of claims 1-13, wherein the patient is suffering from symptomatic obstructive hypertrophic cardiomyopathy.

15. The method of claim 14, wherein the patient is suffering from symptomatic New York Heart Association (NYHA) class II-III obstructive hypertrophic cardiomyopathy.

16. The method of any one of claims 1-15, wherein the myosin inhibitor is selected from the group consisting of a compound of group (I), a compound of group (II), a compound of group (III), mavacamten, MYK-581, aficamten, and pharmaceutically acceptable salts thereof.

17. The method of claim 16, wherein the myosin inhibitor is mavacamten or a pharmaceutically acceptable salt thereof.

18. The method of claim 16, wherein the myosin inhibitor is mavacamten.

19. The method of one of claim 18, wherein the starting dose is about 5 mg per day of mavacamten.

20. The method of claim 19, wherein the first reduced dose is less than about 5 mg per day of mavacamten.

21. The method of claim 20, wherein the first reduced dose is selected from the group consisting of about 2.5 mg per day of mavacamten, about 1 mg per day of mavacamten, or 0 mg per day of mavacamten.

22. The method of claim 21, wherein the second reduced dose is about 1 mg per day of mavacamten or 0 mg per day of mavacamten.

23. The method of claim 19, wherein the first reduced dose is about 2.5 mg per day of mavacamten and the second reduced dose is 0 mg per day of mavacamten.

24. The method of any one of claims 1-23, wherein a left ventricular ejection fraction of the patient at or near the conclusion of the first and second treatment periods is greater than or equal to about 50%.

25. A method of treating symptomatic obstructive hypertrophic cardiomyopathy in a patient in need thereof, comprising:
administering a starting dose of 5 mg per day of mavacamten to the patient for a first treatment period, wherein the first treatment period is about 4 weeks;
administering 2.5 mg per day of mavacamten to the patient for a second treatment period when a Valsalva left ventricular outflow tract (LVOT) gradient of the patient taken at or near the conclusion of the first treatment period is below 20 mmHg, wherein the second treatment period is about 4 weeks; and
administrating 0 mg per day of mavacamten to the patient for a third treatment period when a Valsalva left ventricular outflow tract (LVOT) gradient of the patient taken at or near the conclusion of the second treatment period is below 20 mmHg, wherein the third treatment period is about 4 weeks.

26. The method of claim 25, wherein the second treatment period immediately follows the first treatment period.

27. The method of claim 25 or 26, wherein the third treatment period immediately follows the second treatment period.

28. The method of any one of claims 25-27, further comprising administering 2.5 mg per day of mavacamten to the patient for a fourth treatment period when a measurement of left ventricular ejection fraction of the patient taken at or near the conclusion of the third treatment period is greater than or equal to about 50%, wherein the fourth treatment period is about 4 weeks

29. The method of claim 28, wherein the fourth treatment period immediately follows the third treatment period.

30. The method of any one of claims 25-29, wherein the patient has a LVEF of greater than or equal to about 50%.

31. A method of treating a patient in need thereof with a myosin inhibitor, the method comprising the steps of:
(a) administering a starting dose of the myosin inhibitor during a first treatment period;
(b) assessing the patient for left ventricular outflow tract obstruction to obtain a first assessment outcome and determining whether the first assessment outcome is below a first threshold value;
(c) when the first assessment outcome is below a first threshold value, administering a second dose during a second treatment period, wherein the second dose is less than the starting dose;
(d) assessing the patient for left ventricular outflow tract obstruction to obtain a second assessment outcome and determining whether the second assessment outcome is below a second threshold value; and
(e) when the second assessment outcome is below a second threshold value, administering a third dose during a third treatment period, wherein the third dose is less than the second dose.

32. The method of claim 31, further comprising the steps of:
(f) at or near the conclusion of the third treatment period, assessing the patient for left ventricular outflow tract obstruction to obtain a third assessment outcome and determining whether the third assessment outcome is greater than or equal to a third threshold value, and assessing the left ventricular ejection fraction (LVEF) of the patient; and
(g) when the third assessment outcome is greater than or equal to a third threshold value and the LVEF of the patient is greater than or equal to a LVEF threshold, administering a fourth dose during a fourth treatment period, wherein the fourth dose is greater than the third dose.

33. The method of claim 31, further comprising the steps of:
(f) at or near the conclusion of the third treatment period, assessing the left ventricular ejection fraction (LVEF) of the patient; and
(g) when the LVEF of the patient is greater than or equal to a safety threshold, administering a fourth dose during a fourth treatment period, wherein the fourth dose is greater than the third dose.

34. The method of claim 32 or 33, further comprising the steps of:
(h) at or near the conclusion of the fourth treatment period, assessing the patient for left ventricular outflow tract obstruction to obtain a fourth assessment outcome and determining whether the fourth assessment outcome is greater than or equal to a fourth threshold value, and assessing the left ventricular ejection fraction (LVEF) of the patient; and
(i) when the fourth assessment outcome is greater than or equal to the fourth threshold value and the LVEF of the patient is greater than or equal to a LVEF threshold, administering a fifth dose during a fifth treatment period, wherein the fifth dose is greater than the fourth dose.

35. The method of claim 31, wherein the first and second assessments are performed by a non-invasive technique.

36. The method of claim 32, wherein the third assessment is performed by a non-invasive technique.

37. The method of claim 35 or 36, wherein the non-invasive technique comprises echocardiography

38. The method of claim 35 or 36, wherein the non-invasive technique comprises a cardiac imaging technique.

39. The method of any one of claims 35-38, wherein the non-invasive technique comprises measurement of LVOT gradient with Valsalva maneuver.

40. The method of claim 39, wherein the first assessment outcome is a first Valsalva LVOT gradient and the second assessment outcome is a second Valsalva LVOT gradient.

41. The method of claim 40, wherein the first threshold value and the second threshold value are each a Valsalva LVOT gradient.

42. The method of claim 41, wherein the first threshold value and the second threshold value are each a Valsalva LVOT gradient of 20 mmHg.

43. The method of any one of claims 31-42, wherein the method mitigates the risk of an adverse event.

44. The method of claim 43, wherein the adverse event is systolic dysfunction.

45. The method of claim 43, wherein the adverse event is heart failure.

46. The method of any one of claims 43-45, wherein the patient's risk of the adverse event is reduced as compared to continued administration of the myosin inhibitor at the starting dose.

47. The method of any one of claims 31-46, wherein the myosin inhibitor is selected from the group consisting of a compound of group (I), a compound of group (II), a compound of group (III), mavacamten, MYK-581, and aficamten, and pharmaceutically acceptable salts thereof.

48. The method of claim 47, wherein the myosin inhibitor is mavacamten or a pharmaceutically acceptable salt thereof.

49. The method of claim 47, wherein the myosin inhibitor is mavacamten.

50. The method of claim 49, wherein the starting dose is 5 mg per day of mavacamten.

51. The method of claim 50, wherein the second dose is less than 5 mg per day of mavacamten.

52. The method of claim 51, wherein the second dose is 2.5 mg per day of mavacamten.

53. The method of claim 51, wherein the third dose is less than 2.5 mg per day of mavacamten.

54. The method of claim 52, wherein the third dose is 0 mg per day of mavacamten or 1 mg per day of mavacamten.

55. The method of any one of claims 31-54, wherein the patient is suffering from obstructive hypertrophic cardiomyopathy (oHCM).

56. The method of claim 55, wherein the patient is suffering from symptomatic New York Heart Association (NYHA) class II-III oHCM.

57. The method of claim 31, wherein a dose of 0 mg is administered during the third treatment period.

58. The method of any one of claims 31-57, wherein the dose of the myosin inhibitor administered to the patient is not increased until after the third treatment period.

59. The method of claim 32, wherein the LVEF threshold is 55%.

60. The method of claim 33, wherein the safety threshold is 50%.

61. The method of any one of claims 31-60, further comprising assessing the patient for left ventricular ejection fraction (LVEF) at or near the conclusion of the first treatment period and at or near the conclusion of the second treatment period.

62. The method of claim 61, further comprising temporarily discontinuing treatment when the LVEF assessment at or near the conclusion of the first or second treatment period is less than 50%.

63. The method of any one of claims 31-62, wherein the first treatment period is about four weeks and the second treatment period is about four weeks.

64. The method of claim 63, wherein the first treatment period is about four weeks, the second treatment period is about four weeks, and the third treatment period is about four weeks.

65. A method of treating a patient in need thereof with mavacamten, the method comprising the steps of:
(a) administering 5 mg per day of mavacamten to the patient during a first treatment period;
(b) assessing the patient for LVOT gradient with Valsalva maneuver to determine a first Valsalva LVOT gradient;
(c) administering 2.5 mg per day of mavacamten per day to the patient during a second treatment period when the first Valsalva LVOT gradient is below 20 mmHg;
(d) assessing the patient for LVOT gradient with Valsalva maneuver to determine a second Valsalva LVOT gradient; and
(e) administering 0 mg or 1 mg of mavacamten per day to the patient for a third treatment period when the second Valsalva LVOT gradient is below 20 mmHg.

66. The method of claim 65, further comprising the steps of:
(f) at or near the conclusion of the third treatment period, assessing the patient for LVOT gradient with Valsalva maneuver to determine a third Valsalva LVOT gradient and assessing the left ventricular ejection fraction (LVEF) of the patient; and
(g) administering 2.5 mg of mavacamten per day to the patient for a fourth treatment period when the third Valsalva LVOT gradient is greater than or equal to 30 mmHg and the LVEF of the patient is greater than or equal to 55%.

67. The method of claim 65, further comprising the steps of:
(f) at or near the conclusion of the third treatment period, assessing the left ventricular ejection fraction (LVEF) of the patient; and
(g) administering 2.5 mg of mavacamten per day to the patient for a fourth treatment period when the LVEF of the patient is greater than or equal to 50%.

68. The method of claim 66 or 67, further comprising the steps of:
(h) at or near the conclusion of the fourth treatment period, assessing the patient for LVOT gradient with Valsalva maneuver to determine a fourth Valsalva LVOT gradient and assessing the left ventricular ejection fraction (LVEF) of the patient; and
(i) administering 5 mg of mavacamten per day to the patient for a fifth treatment period when the fourth Valsalva LVOT gradient is greater than or equal to 30 mmHg and the LVEF of the patient is greater than or equal to 55%.

69. A method of administering mavacamten to a patient, wherein the patient is suffering from oHCM, comprising the steps of:
(a) administering to the patient a starting dose of 5 mg per day of mavacamten for a first treatment period;
(b) assessing the patient for LVOT gradient with Valsalva maneuver to determine a first Valsalva LVOT gradient;
(c) administering to the patient 2.5 mg per day of mavacamten for a second treatment period when the first Valsalva LVOT gradient is less than 20 mmHg;
(d) assessing the patient for LVOT gradient with Valsalva maneuver to determine a second Valsalva LVOT gradient;
(e) administering to the patient 0 mg per day of mavacamten for a third treatment period when the second Valsalva LVOT gradient is less than 20 mmHg;
(f) assessing the patient to determine a first left ventricular ejection fraction (LVEF); and
(g) administering to the patient 2.5 mg per day of mavacamten for a fourth treatment period when the first LVEF is greater than or equal to 50%.

70. The method of claim 69, further comprising the steps of:
(h) assessing the patient for LVOT gradient with Valsalva maneuver to determine a third Valsalva LVOT gradient and assessing the patient to determine a second left ventricular ejection fraction (LVEF); and
(i) administering to the patient 5 mg per day of mavacamten for a fifth treatment period when the third Valsalva LVOT gradient is greater than or equal to 30 mmHg and the second LVEF is greater than or equal to 55%.

71. The method of claim 69 or 70, wherein a risk of systolic dysfunction and/or heart failure in the patient is reduced as compared to continued administration of the myosin inhibitor at the starting dose.

72. The method of any of claims 69-71, wherein the first treatment period is about four weeks and the second treatment period is about four weeks.

73. The method claim 72, wherein the third treatment period is about four weeks.

74. The method of claim 73, wherein the fourth treatment period is about twelve weeks.

75. The method of any one of claims 69-74, wherein the patient is suffering from symptomatic New York Heart Association (NYHA) class II-III oHCM.

76. A method of administering mavacamten to a patient, wherein the patient is suffering from oHCM, comprising the steps of:
administering to the patient a starting dose of 5 mg per day of mavacamten for a first treatment period;
assessing the patient for LVOT gradient with Valsalva maneuver to determine a first Valsalva LVOT gradient;
administering a second dose of mavacamten during a second treatment period, wherein if the first Valsalva LVOT gradient is less than 20 mmHg, then the second dose is 2.5 mg per day, and wherein if the first Valsalva LVOT gradient is greater than or equal to 20 mmHg, then the second dose is 5 mg per day;
assessing the patient for LVOT gradient with Valsalva maneuver to determine a second Valsalva LVOT gradient;
administering a third dose of mavacamten during a third treatment period, wherein if the second Valsalva LVOT gradient is less than 20 mmHg, then the third dose is less than the second dose and the third dose is 2.5 mg, 1 mg, or 0 mg per day; and wherein if the first Valsalva LVOT gradient is greater than or equal to 20 mmHg, then the third dose is the same as the second dose and the third dose is 5 mg or 2.5 mg per day.

77. The method of claim 76, wherein the patient receives a third dose of 0 mg per day during the third treatment period; the method further comprising the steps of:
assessing the patient to determine a left ventricular ejection fraction (LVEF); and
administering a fourth dose of mavacamten during a fourth treatment period, wherein if the LVEF is greater than or equal to 50%, then the fourth dose is is 2.5 mg per day, and wherein if the LVEF is less than 50%, then the fourth dose is 0 mg per day.

78. The method of claim 76, wherein the patient receives a third dose of 1 mg per day, 2.5 mg per day, or 5 mg per day during the third treatment period; the method further comprising the steps of:
assessing the patient for LVOT gradient with Valsalva maneuver to determine a third Valsalva LVOT gradient and assessing the patient to determine a left ventricular ejection fraction (LVEF); and
administering a fourth dose of mavacamten during a fourth treatment period, wherein if the third Valsalva LVOT gradient is greater than or equal to 30 mmHg and the LVEF is greater than or equal to 55%, then the fourth dose is greater than the third dose and the fourth dose is 2.5 mg, 5 mg, or 10 mg per day, and wherein if the third Valsalva LVOT gradient is less than 30 mmHg or the LVEF is less than 55%, then the fourth dose is the same as the third dose and the fourth dose is 1 mg, 2.5 mg, or 5 mg per day.

79. The method of any one of claims 76-78, wherein a risk of systolic dysfunction and/or heart failure in the patient is reduced as compared to if the patient received continued administration of mavacamten at the starting dose.

80. The method of any one of claims 76-79, wherein the first treatment period is about four weeks and the second treatment period is about four weeks.

81. The method claim 80, wherein the third treatment period is about four weeks.

82. The method of claim 81, wherein the fourth treatment period is about twelve weeks.

83. The method of any one of claims 76-82, wherein the patient is suffering from symptomatic New York Heart Association (NYHA) class II-III oHCM.

84. A method of treating a patient in need thereof with a myosin inhibitor, comprising:
administering a starting dose of a myosin inhibitor to the patient for a first treatment period;
administering a second dose of the myosin inhibitor to the patient for a second treatment period, wherein:
if a LVOT gradient of the patient taken at or near the conclusion of the first treatment period is below a threshold value, then the second dose is less than the starting dose, and
if a LVOT gradient of the patient taken at or near the conclusion of the first treatment period is greater than or equal to the threshold value, then the second dose is the same as the starting dose; and
administering a third dose of the myosin inhibitor to the patient for a third treatment period, wherein:
if a LVOT gradient of the patient taken at or near the conclusion of the second treatment period is below the threshold value, then the third dose is less than the second dose, and
if a LVOT gradient of the patient taken at or near the conclusion of the second treatment period is greater than or equal to the threshold value, then the third dose is the same as the second dose.

85. The method of claim 84, wherein the second treatment period immediately follows the third treatment period.

86. The method of claim 85, wherein the third treatment period immediately follows the second treatment period.

87. The method of any one of claims 84-86, further comprising administering a fourth dose of the myosin inhibitor to the patient for a fourth treatment period, wherein:
if the third dose is less than the second dose, and the second dose is less than the starting dose, and a measurement of left ventricular ejection fraction of the patient taken at or near the conclusion of the third treatment period is greater than or equal to about 50%, then the fourth dose is the same as the lowest previously administered dose and the fourth treatment period, and
if the third dose is equal to the second dose and/or the second dose is equal to the starting dose, then the fourth treatment period is longer than the third treatment period.

88. The method of claim 87, wherein the fourth treatment period immediately follows the third treatment period.

89. The method of any one of claims 84-88, wherein the first, second, and third treatment periods are about four weeks.

90. The method of any one of claims 84-89, wherein the threshold value is 20 mmHg.

91. The method of any one of claims 84-90, wherein the fourth treatment period is about 4 weeks when the third dose is less than the second dose, and the second dose is less than the starting dose, and a measurement of LVEF of the patient taken at or near the conclusion of the third treatment period is greater than or equal to about 50%.

92. The method of any one of claims 84-90, wherein the fourth treatment period is about 12 weeks when the third dose is equal to the second dose and/or the second dose is equal to the starting dose.

93. The method of any one of claims 84-92, wherein the patient has a LVEF of greater than or equal to about 50%.

94. The method of any one of claims 84-93, where the LVOT gradient is a Valsalva LVOT gradient.

95. The method of any one of claims 84-94, wherein the patient's risk of an adverse event is reduced as compared to continued administration of the myosin inhibitor at the starting dose.

96. The method of any one of claims 84-95, wherein the first, second, and third treatment periods are each about 4 weeks.

97. The method of any one of claims 84-96, wherein the patient is suffering from symptomatic obstructive hypertrophic cardiomyopathy.

98. The method of claim 97, wherein the patient is suffering from symptomatic New York Heart Association (NYHA) class II-III obstructive hypertrophic cardiomyopathy.

99. The method of any one of claims 84-98, wherein the myosin inhibitor is selected from the group consisting of a compound of group (I), a compound of group (II), a compound of group (III), mavacamten, MYK-581, aficamten, and pharmaceutically acceptable salts thereof.

100. The method of claim 99, wherein the myosin inhibitor is mavacamten or a pharmaceutically acceptable salt thereof.

101. The method of claim 99, wherein the myosin inhibitor is mavacamten.

102. The method of claim 101, wherein the starting dose is about 5 mg per day of mavacamten.

103. The method of claim 102, wherein the second dose is less than about 5 mg per day of mavacamten, or wherein the second dose is selected from the group consisting of about 2.5 mg per day of mavacamten, about 1 mg per day of mavacamten, or 0 mg per day of mavacamten.

104. The method of claim 103, wherein the third dose is less than about 2.5 mg per day of mavacamten or wherein the third dose is 1 mg per day or 0 mg per day.

105. The method of claim 104, wherein the second dose is about 2.5 mg per day of mavacamten and the third dose is 0 mg per day of mavacamten.

106. The method of any one of claims 84-105, wherein the fourth dose is selected from the group consisting of 2.5 mg, 5 mg, and 10 mg per day of mavacamten.

107. A method of treating a patient in need thereof with a myosin inhibitor, the method comprising:
administering a starting dose of the myosin inhibitor at least once per day at the start of an initiation phase; and
performing one or more assessments of the patient for left ventricular outflow tract obstruction during the initiation phase to obtain one or more assessment outcomes; and
discontinuing administration of the myosin inhibitor based on the one or more assessment outcomes.

108. The method of claim 107, further comprising resuming administration of the myosin inhibitor after the discontinuation.

109. The method of claim 108, wherein administration is resumed following an assessment of LVEF of the patient, wherein administration is resumed when LVEF is greater than or equal to a safety threshold.

110. The method of any one of claims 107-109, wherein the one or more assessments are performed by a non-invasive technique.

111. The method of claim 110, wherein the non-invasive technique comprises echocardiography.

112. The method of claim 110, wherein the non-invasive technique comprises a cardiac imaging technique.

113. The method of claim 110, wherein the non-invasive technique comprises measurement of LVOT gradient with Valsalva maneuver and the one or more assessment outcomes are one or more Valsalva LVOT gradients.

114. The method of claim 113, comprising discontinuing administration of the myosin inhibitor when a Valsalva LVOT gradient is below 20 mmHg.

115. The method of any one of claims 110-114, comprising performing two or more assessments of the patient for left ventricular outflow obstruction by a non-invasive technique during the initiation phase to obtain two or more assessment outcomes.

116. The method of claim 115, wherein the non-invasive technique comprises measurement of LVOT gradient with Valsalva maneuver and the two or more assessment outcomes are two or more Valsalva LVOT gradients.

117. The method of claim 116, comprising discontinuing administration of the myosin inhibitor when at least two of the two or more Valsalva LVOT gradients are below 20 mmHg.

118. The method of any one of claims 107-117, wherein the method mitigates the patient's risk of an adverse event.

119. The method of claim 118, wherein the adverse event is systolic dysfunction.

120. The method of claim 118, wherein the adverse event is heart failure.

121. The method of any one of claims 118-120, wherein the patient's risk of the adverse event is reduced as compared to continued administration of the myosin inhibitor.

122. The method of any one of claims 107-121, wherein the myosin inhibitor is selected from the group consisting of a compound of group (I), a compound of group (II), a compound of group (III), mavacamten, MYK-581, and aficamten, optionally as a pharmaceutically acceptable salt thereof.

123. The method of claim 122, wherein the myosin inhibitor is mavacamten or a pharmaceutically acceptable salt thereof.

124. The method of claim 122, wherein the myosin inhibitor is mavacamten.

125. The method of claim 124, wherein the starting dose is 5 mg per day of mavacamten.

126. The method of any one of claims 107-125, wherein the patient is suffering from oHCM.

127. The method of any one of claims 107-126, wherein the initiation phase is from about 4 weeks to about 6 months in duration.

128. The method of claim 127, wherein the initiation phase is from about 8 weeks to about 16 weeks in duration.

129. The method of claim 109, wherein the safety threshold is 50%.

130. A method of treating a patient in need thereof with mavacamten, comprising the steps of:
(a) administering a starting dose of 5 mg per day of mavacamten to the patient at the start of an initiation phase; and
(b) performing two or more assessments of the patient for LVOT gradient with Valsalva maneuver at separate times during the initiation phase to obtain two or more Valsalva LVOT gradients; and
(c) discontinuing administration of mavacamten when each of the two or more Valsalva LVOT gradients is below 20 mmHg.

131. A method of treating a patient in need thereof with a myosin inhibitor, the method comprising:
administering a starting dose of the myosin inhibitor to the patient;
assessing the patient for left ventricular outflow tract obstruction at or near the conclusion of two or more separate treatment periods to obtain two or more assessment outcomes; and
administering a first reduced dose and subsequently administering a second reduced dose, based upon the two or more assessment outcomes, wherein said assessment outcomes are below a threshold value, wherein the first reduced dose is less than the starting dose, and the second reduced dose is less than the first reduced dose.

132. The method of claim 131, wherein the dose of the myosin inhibitor administered to the patient is not increased until the two or more assessment outcomes are completed at or near the conclusion of the two or more separate treatment periods.

133. The method of claim 131 or 132, wherein the two or more assessments are performed by a non-invasive technique.

134. The method of claim 133, wherein the non-invasive technique comprises echocardiography.

135. The method of claim 133, wherein the non-invasive technique comprises a cardiac imaging technique.

136. The method of any one of claims 133-135, wherein the non-invasive technique comprises measurement of LVOT gradient with Valsalva maneuver.

137. The method of claim 136, wherein the assessment outcome is a Valsalva LVOT gradient.

138. The method of claim 137, wherein the threshold value is a Valsalva LVOT gradient.

139. The method of claim 138, wherein the threshold value is a Valsalva LVOT gradient of 20 mmHg.

140. The method of any one of claims 131-139, wherein the method mitigates the risk of an adverse event.

141. The method of claim 140, wherein the adverse event is systolic dysfunction.

142. The method of claim 140, wherein the adverse event is heart failure.

143. The method of any one of claims 140-142, wherein the patient's risk of the adverse event is reduced as compared to continued administration of the myosin inhibitor at the starting dose.

144. The method of any one of claims 131-143, wherein the myosin inhibitor is selected from the group consisting of a compound of group (I), a compound of group (II), a compound of group (III), mavacamten, MYK-581, aficamten, and pharmaceutically acceptable salts thereof.

145. The method of claim 144, wherein the myosin inhibitor is mavacamten or a pharmaceutically acceptable salt thereof.

146. The method of claim 144, wherein the myosin inhibitor is mavacamten.

147. The method of claim 146, wherein the starting dose is 5 mg per day of mavacamten.

148. The method of claim 147, wherein the first reduced dose is less than 5 mg per day.

149. The method of claim 148, wherein the second reduced dose is less than 2.5 mg per day.

150. The method of claim 148, wherein the first reduced dose is 2.5 mg per day of mavacamten.

151. The method of claim 150, wherein the second reduced dose is 1 mg per day or 0 mg per day of mavacamten.

152. The method of any one of claims 131-151, wherein the patient is suffering from obstructive hypertrophic cardiomyopathy (oHCM).

153. The method of claim 152, wherein the patient is suffering from symptomatic New York Heart Association (NYHA) class II-III oHCM.

154. The method of any one of claims 131-153, wherein the two or more separate treatment periods comprise a first treatment period, and a second treatment period, and wherein the two or more assessment outcomes comprise a first assessment outcome at or near the conclusion of the first treatment period and a second assessment outcome at or near the conclusion of the second treatment period.

155. The method of claim 154, further comprising administering the myosin inhibitor for a third treatment period, following the second assessment.

156. The method of claim 154 or 155, wherein the dose of the myosin inhibitor administered to the patient is not increased until after the third treatment period.

157. The method of any one of claims 154-156, wherein the first treatment period is about four weeks and the second treatment period is about four weeks.

158. The method of claim 157, wherein the third treatment period is about four weeks.

159. The method of any one of claims 131-158, further comprising assessing the left ventricular ejection fraction (LVEF) of the patient at or near the conclusion of the two or more separate treatment periods.

160. A method of mitigating a risk of heart failure with reduced ejection fraction due to administration of a myosin inhibitor to a patient, the method comprising the steps of:
administering a myosin inhibitor to the patient;
temporarily discontinuing administration of the myosin inhibitor when the patient has a LVEF of less than 50%;
resuming administration of the myosin inhibitor to the patient when the patient has a LVEF of greater than or equal to 50%; and
permanently discontinuing administration of the myosin inhibitor when the patient has a LVEF of less than 50% after resuming administration.

161. The method of claim 160, wherein the LVEF is determined by a non-invasive technique.

162. The method of claim 161, wherein the non-invasive technique is echocardiography.

163. The method of claim 161, wherein the non-invasive technique comprises a cardiac imaging technique.

164. The method of any one of claims 160-163, wherein resuming administration comprises administering the same dose that the patient received prior to temporary discontinuation.

165. The method of any one of claims 160-163, wherein resuming administration comprises administering a lower dose than the dose the patient received prior to temporary discontinuation.

166. The method of any one of claims 160-163, wherein resuming administration comprises administering a minimum dose of myosin inhibitor to the patient, wherein the minimum dose is the lowest dose of the myosin inhibitor approved to be administered to patients by a governmental regulatory agency.

167. The method of claim 166, wherein the governmental regulatory agency is an agency of the United States, European Union, Switzerland, Japan, China, South Korea, Canada, Mexico, Australia, New Zealand, Brazil, Russia, Ukraine, Georgia, Vietnam, Singapore, Malaysia, Philippines, India, Indonesia, Hong Kong, Israel, South Africa, Colombia, Costa Rica, Dominican Republic, Ecuador, Guatemala, El Salvador, Honduras, Egypt, Syria, Algeria, Kenya, Morocco, or Nigeria.

168. The method of any one of claims 160-167, wherein the myosin inhibitor is selected from the group consisting of a compound of group (I), a compound of group (II), a compound of group (III), mavacamten, MYK-581, and aficamten, and pharmaceutically acceptable salts thereof.

169. The method of claim 168, wherein the myosin inhibitor is mavacamten or a pharmaceutically acceptable salt thereof.

170. The method of claim 168, wherein the myosin inhibitor is mavacamten.

171. The method of any one of claims 160-170, wherein the patient is suffering from obstructive hypertrophic cardiomyopathy (oHCM).

172. The method of claim 171, wherein the patient is suffering from symptomatic New York Heart Association (NYHA) class II-III oHCM.

173. A method of mitigating a risk of heart failure with reduced ejection fraction due to administration of mavacamten to a patient, the method comprising the steps of:
administering mavacamten to the patient at a dose of 2.5 mg per day;
temporarily discontinuing administration of mavacamten when the patient has a LVEF of less than 50%;
resuming administration of mavacamten to the patient at a dose of 2.5 mg per day when the patient has a LVEF of greater than or equal to 50%; and
permanently discontinuing administration of mavacamten when the patient has a LVEF of less than 50% after resuming administration.

174. A method of treating obstructive hypertrophic cardiomyopathy (oHCM) in a patient in need thereof, the method comprising administering a therapeutically effective amount of mavacamten to the patient, wherein the patient does not receive concomitant administration of a strong or moderate CYP2C19 inducer nor a strong or moderate CYP3A4 inducer.

175. A method of treating obstructive hypertrophic cardiomyopathy (oHCM) in a patient in need thereof, where the patient is being treated with a strong or moderate CYP2C19 inducer or a strong or moderate CYP3A4 inducer, the method comprising:
discontinuing administration to the patient of the strong or moderate CYP2C19 inducer or strong or moderate CYP3A4 inducer; and
administering a therapeutically effective amount of mavacamten to the patient, thereby avoiding the use of mavacamten in combination with a strong or moderate CYP2C19 inducer or a strong or moderate CYP3A4 inducer.

176. A method of administering a myosin inhibitor to a patient who initiates concomitant therapy with a weak CYP2C19 inhibitor or a moderate CYP3A4 inhibitor while receiving myosin inhibitor therapy, the method comprising:
administering a first daily dose of the myosin inhibitor during a first treatment period prior to initiating concomitant therapy with a weak CYP2C19 inhibitor or a moderate CYP3A4 inhibitor;
administering a second daily dose of the myosin inhibitor, which is less than the first daily dose, during a second treatment period, wherein the patient receives concomitant therapy with a weak CYP2C19 inhibitor or a moderate CYP3A4 inhibitor during the second treatment period.

177. The method of claim 176, further comprising assessing LVEF of the patient during the second treatment period and temporarily discontinuing administration of the myosin inhibitor if LVEF is below a safety threshold.

178. The method of claim 177, wherein the safety threshold is 50%.

179. The method of claim 177, further comprising assessing LVEF and LVOT gradient of the patient after discontinuing administration, and resuming administration of the first daily dose when the LVOT gradient is greater than or equal to a threshold value and the LVEF is greater than or equal to a LVEF threshold.

180. The method of claim 179, wherein the threshold value is 30 mmHg and the LVEF threshold is 55%.

181. The method of any one of claims 176-180, wherein the myosin inhibitor is selected from the group consisting of a compound of group (I), a compound of group (II), a compound of group (III), mavacamten, MYK-581, and aficamten, and pharmaceutically acceptable salts thereof.

182. The method of claim 181, wherein the myosin inhibitor is mavacamten or a pharmaceutically acceptable salt thereof.

183. The method of claim 181, wherein the myosin inhibitor is mavacamten.

184. The method of claim 183, wherein the first daily dose is 5 mg, 10 mg, or 15 mg of mavacamten, and the second daily dose is 2.5 mg, 5 mg, or 10 mg of mavacamten.

185. The method of any one of claims 176-184, wherein the weak CYP2C19 inhibitor or moderate CYP3A4 inhibitor is selected from the group consisting of cimetidine, ciprofloxacin, diltiazem, felbamate, omeprazole at a dose of 20 mg once daily, isoniazid, fluconazole, and verapamil.

186. The method of any one of claims 176-185, wherein the patient is suffering from obstructive hypertrophic cardiomyopathy (oHCM).

187. The method of claim 186, wherein the patient is suffering from symptomatic New York Heart Association (NYHA) class II-III oHCM.

188. The method of claim 177, wherein assessing LVEF of the patient during the second treatment period comprises assessing LVEF of the patient about four weeks after beginning the concomitant therapy.

189. The method of any one of claims 176-188, wherein the second treatment period is at least 12 weeks, and wherein the second daily dose is not increased to a higher dose during at least the first 12 weeks of the second treatment period.

190. A method of treating HCM in a patient being administered a first daily dose of mavacamten, wherein said patient is then in need of being treated concurrently with a weak CYP2C19 inhibitor or a moderate CYP3A4 inhibitor in addition to the mavacamten, comprising:
administering to the patient a second daily dose of mavacamten, which is less than the first daily dose, in addition to administration of the weak CYP2C19 inhibitor or moderate CYP3A4 inhibitor.

191. The method of claim 190, wherein the first daily dose is 5 mg, 10 mg, or 15 mg per day and the second daily dose is 2.5 mg, 5 mg, or 10 mg per day.

192. A method of initiating concomitant administeration of mavacamten to a patient being administered a weak CYP2C19 inhibitor or a moderate CYP3A4 inhibitor, wherein the patient is in need of concomitant administration of mavacamten and the weak CYP2C19 inhibitor or the moderate CYP3A4 inhibitor and wherein the patient is on a stable therapy of the weak CYP2C19 inhibitor or the moderate CYP3A4 inhibitor, the method comprising:
concomitantly administering a daily dose of 5 mg per day of mavacamten and the stable therapy of the weak CYP2C19 inhibitor or the moderate CYP3A4 inhibitor to the patient.

193. A method of administering a myosin inhibitor to a patient who initiates or increases the dose of a concomitant therapy with a negative inotrope while receiving myosin inhibitor therapy, the method comprising:
(a) administering a therapeutically effective amount of a myosin inhibitor during a first treatment period;
(b) continuing to administer the myosin inhibitor, during a second treatment period, wherein the patient initiates or increases the dose of a concomitant therapy with a negative inotrope during the second treatment period; and
(c) providing echocardiographic monitoring of LVEF during the second treatment period.

194. The method of claim 193, wherein echocardiographic monitoring of LVEF is provided until stable doses and clinical response have been achieved.

195. The method of claim 193 or 194, further comprising providing close medical supervision during the second treatment period.

196. The method of any one of claims 193-195, wherein the myosin inhibitor is mavacamten.

197. A computer-implemented method when executed by data processing hardware causes the data processing hardware to perform operations comprising:
obtaining a healthcare professional (HCP) assessment record associated with a patient, the HCP assessment record comprising a procedure result;
determining, using the HCP assessment record, whether the patient is authorized to receive a prescription authorization authorizing use of a prescription medication; and
when the patient is authorized to receive the prescription medication:
obtaining a pharmacy assessment record associated with the patient, the pharmacy assessment record comprising a medical condition of the patient;
determining, using the pharmacy assessment record, whether a pharmacy is authorized to dispense the prescription medication to the patient; and
when the pharmacy is authorized to dispense the prescription medication to the patient:
generating a dispensation authorization; and
transmitting the dispensation authorization to the pharmacy.

198. The method of claim 197, wherein the procedure result comprises an echocardiogram result.

199. The method of claim 197 or 198, wherein at least one of the HCP assessment record and the pharmacy assessment record comprises potential drug-drug interactions.

200. The method of any one of claims 197-199, wherein obtaining the HCP assessment record comprises retrieving the HCP assessment record from a database remote from the data processing hardware.

201. The method of any one of claims 197-200, wherein determining whether the patient is authorized to receive the prescription authorization is in response to receiving a prescription request.

202. The method of any one of claims 197-201, wherein the dispensation authorization comprises:
a quantity of the prescription medication the patient is authorized to receive; and
a period of time the patient is authorized to receive the prescription medication.

203. The method of any one of claims 197-202, wherein the operations further comprise, after the period of time has elapsed:
determining whether an updated procedure result associated with the patient is available;
when the updated procedure result is available, updating, using the updated procedure result, the dispensation authorization; and
when the updated procedure result is unavailable, declining to update the dispensation authorization.

204. The method of any one of claims 197-203, wherein updating the dispensation authorization comprises at least one of:
adjusting the quantity of the prescription medication the patient is authorized to receive; and
adjusting the period of time the patient is authorized to receive the prescription medication.

205. The method of any one of claims 197-204, wherein the operations further comprise, when the patient is not authorized to receive the prescription medication or when the pharmacy is not authorized to dispense the prescription medication to the patient, generating a report for a regulatory agency.

206. The method of any one of claims 197-205, wherein the prescription medication comprises mavacamten.

207. A system comprising:
data processing hardware; and
memory hardware in communication with the data processing hardware, the memory hardware storing instructions that when executed on the data processing hardware cause the data processing hardware to perform operations recited in any of method claims 197-206.

208. A method of mitigating a risk of heart failure due to systolic dysfunction in a patient being administered a myosin inhibitor, comprising:
providing a data storage facility comprising a database comprising patient HCP assessment records and patient pharmacy assessment records, wherein each patient HCP assessment record comprises information on the patient's date of an echocardiogram, LVEF determined from the echocardiogram, VLVOT determined from the echocardiogram, experience of a clinical heart failure event, and risk of potential drug-drug interactions, and wherein each patient pharmacy assessment record comprises information on the patient's medical conditions, concomitant medications and supplements, and potential drug-drug interactions;
providing a central controller having one or more processors coupled to a communication network, which central controller is coupled to the data storage facility to read and write data to the data storage facility via the network, wherein:
the central controller controls transmission and receipt of data to and from the data storage facility via the network,
the central controller being programed to output via the network a HCP authorization for prescription of the myosin inhibitor to the patient, wherein output of the HCP authorization is dependent upon satisfactory HCP information on the date of echocardiogram, the echocardiogram outcomes, the experience of a clinical heart failure event, and the risk of drug-drug interactions entered into each patient HCP assessment record, and wherein the central controller inhibits the HCP authorization output for unsatisfactory HCP information,
the central controller being further programed to output via the network a dispensation authorization for the myosin inhibitor to the patient, wherein output of the pharmacy authorization is dependent upon output of the HCP authorization and satisfactory pharmacy information on the patient's medical conditions, concomitant medications and supplements, and potential drug-drug interactions entered into each patient pharmacy assessment record, and wherein the central controller inhibits the dispensation authorization output for unsatisfactory pharmacy information and/or lack of HCP authorization, and
wherein the central controller manages one or more aspects reporting unsatisfactory information on the experience of a clinical heart failure event to a regulatory agency, or other overseeing body.

209. A computer-implemented method when executed by data processing hardware causes the data processing hardware to perform operations comprising:
obtaining a healthcare professional (HCP) assessment record associated with a patient, the HCP assessment record comprising a procedure result;
obtaining a pharmacy assessment record associated with the patient, the pharmacy assessment record comprising a medical condition of the patient;
determining, using the HCP assessment record, whether the patient is authorized to receive a prescription authorization authorizing use of a prescription medication; and
when the patient is authorized to receive the prescription medication:
determining, using the pharmacy assessment record, whether a pharmacy is authorized to dispense the prescription medication to the patient; and
when the pharmacy is authorized to dispense the prescription medication to the patient:
generating a dispensation authorization; and
transmitting the dispensation authorization to the pharmacy.

210. The method of any preceding claim, wherein the procedure result comprises an echocardiogram result.

211. The method of any preceding claim, wherein at least one of the HCP assessment record and the pharmacy assessment record comprises potential drug-drug interactions.

212. The method of any preceding claim, wherein obtaining the HCP assessment record comprises retrieving the HCP assessment record from a database remote from the data processing hardware.

213. The method of any preceding claim, wherein determining whether the patient is authorized to receive the prescription authorization is in response to receiving a prescription request.

214. The method of any preceding claim, wherein the dispensation authorization comprises:
a quantity of the prescription medication the patient is authorized to receive; and
a period of time the patient is authorized to receive the prescription medication.

215. The method of any preceding claim, wherein the operations further comprise, after the period of time has elapsed:
determining whether an updated procedure result associated with the patient is available;
when the updated procedure result is available, updating, using the updated procedure result, the dispensation authorization; and
when the updated procedure result is unavailable, declining to update the dispensation authorization.

216. The method of any preceding claim, wherein updating the dispensation authorization comprises at least one of:
adjusting the quantity of the prescription medication the patient is authorized to receive; and
adjusting the period of time the patient is authorized to receive the prescription medication.

217. The method of any preceding claim, wherein the operations further comprise, when the patient is not authorized to receive the prescription medication or when the pharmacy is not authorized to dispense the prescription medication to the patient, generating a report for a regulatory agency.

218. The method of any preceding claim, wherein the prescription medication comprises mavacamten.

219. A system comprising:
data processing hardware; and
memory hardware in communication with the data processing hardware, the memory hardware storing instructions that when executed on the data processing hardware cause the data processing hardware to the perform operations recited in any of method claims 209-218.
